(19) United States
(12) Patent Application Publication
(10) Pub. No.: US 2026/0265739 A1
(43) Pub. Date: Sep. 10, 2026

(54) Oligonucleotide Modulators Activating Factor VII Expression and Use Thereof in Treatment of Hemophilia

(71) Applicant: Ractigen Therapeutics, Nantong (CN)

(72) Inventors: Longcheng Li, Jiangsu (CN); Moorim Kang, Jiangsu (CN)

(21) Appl. No.: 19/162,163

(22) PCT Filed: Mar. 5, 2024
(86) PCT No.: PCT/CN2024/080083

§ 371 Date: Sep. 4, 2025

(30) Foreign Application Priority Data
Mar. 6, 2023 (WO) PCT/CN2023/079893

(51) Int. Cl. C12N 15/113; A61K 31/7115; A61K 31/712; A61K 31/7125; A61K 31/713; A61K 38/48; A61P 7/04; C12Q 1/37

(52) U.S. Cl. CPC C12N 15/113; A61K 31/7115; A61K 31/712; A61K 31/7125; A61K 31/713; A61K 38/4846; A61P 7/04; C12Q 1/37; C12Y 304/21021; C12N 2310/11; C12N 2310/315; C12N 2310/321; C12N 2310/322; C12N 2310/3341; C12N 2310/351; G01N 2333/96458

(57) ABSTRACT

Provided are saRNAs and oligonucleotide modulators for preventing or treating FVII-related disease or condition or disorder, such as those caused by or associated with insufficient expression of FVII gene or hemorrhagic complications due to hemophilia with inhibitor and use thereof. Also provided are pharmaceutical composition comprising the oligonucleotide modulator and methods for preventing or treating FVII-related disease or condition or disorder induced by insufficient FVII level with the oligonucleotide modulator.

### CROSS-REFERENCE TO RELATED APPLICATIONS

This application claims priority to the filing date of Provisional Patent Application Serial No. PCT/CN2023/079893 filed Mar. 6, 2023, the disclosure of which application is herein incorporated by reference.

### TECHNICAL FIELD

The present application relates to the technical field of nucleic acids, specifically relates to oligonucleotide modulators associated with activation of gene expression and pharmaceutical use thereof.

### SEQUENCE LISTING

The instant application contains a Sequence Listing which has been submitted electronically in computer readable format and is hereby incorporated by reference in its entirety.

### BACKGROUND

Factor VII (FVII, also known as proconvertin), is a clotting factor of the coagulation system and synthesized exclusively by the liver as a serine protease and presents in plasma with extremely short half-life (4-6 hours) at a concentration of 10 nM (0.5 μg/ml) [Heinz S, et. al. 2015; Yang L, et. al. 2016]. Factor VII is the only clotting factor that has a small proportion (1%-3%) of free circulating activated form (FVIIa) in the absence of coagulation activation. Following injury, the integral membrane protein tissue factor (TF) is exposed into the vascular lumen and can bind the free circulating FVIIa in order to trigger coagulation. The action of the FVIIa-TF complex generates a burst of activated factors IX (FIXa) and X (FXa), which results in the formation of stable fibrin clot.

FVII gene harbors more than 200 different variants, including missense, nonsense, small insertion/deletion, and splice site mutations, which may affect every region of the gene. The mutations identified throughout FVII gene can affect all the protein domains. Point mutations are the leading causes of FVII inherited defects, where missense mutations are most frequent. Exon 8 is the largest exon of the gene and harbors a large number of mutations.

FVII-related diseases or disorders or conditions may comprise but not limited to congenital FVII deficiency (Alexander's Disease), Acquired FVII deficiency (AFVIID), hemophilia (such as hemophilia with inhibitor), Glanzmann's thrombasthenia (GT).

Congenital FVII deficiency is a rare autosomal recessive bleeding disorder resulting from variants in the gene encoding FVII. People with severe FVII deficiency experience joint and muscle bleeds, easy bruising and bleeds after surgery.

Acquired FVII deficiency (AFVIID) could be due to an abnormal or decreased synthesis, an accelerated consumption or catabolism, a neutralization by an antibody, or an abnormal absorption by tumor mass. AFVIID can be isolated or combined with other decreased coagulation factor levels. AFVIID is more common than the inherited form.

Hemophilia is an inherited disorder in which the blood does not clot due to insufficient clotting factors. This causes unexplained bleeding, pain, swelling or tightness in joints, blood in urine or stool, and nose bleeds. Inhibitors (also known as antibodies) are one of the most serious complications of hemophilia, and occur when the body thinks the infused factor product is a threat. Recombinant human FVIIa (rhFVIIa) is typically used for on-demand bleeding episode treatment, and also for prophylactic treatment to maintain hemostasis in patients with hemophilia.

Glanzmann's thrombasthenia (GT) is a genetic platelet surface receptor disorder of GPIIb/IIIa (ITG αIIbβ3), either qualitative or quantitative, which results in faulty platelet aggregation and diminished clot retraction. Control and prevention of bleeding among patients with GT is imperative, and remains challenging. Local measures, including anti-fibrinolytic therapy, with or without platelet transfusions, used to be the mainstay of therapy. However, in recent years the use of rhFVIIa has increased significantly, with excellent response rates in treating and preventing hemorrhage among GT patients.

Fresh plasma and recombinant factor VII (rFVII, such as rhFVIIa) are the treatment choices for coagulation factor related diseases or disorders or conditions but need frequent intravenous (IV) infusion with the very short half-life (4~6 hours) and the risk of thrombosis. Moreover, in patients suffering from hemophilia, the infused factor product may induce the production of inhibitors and thus causing hemophilia with inhibitor or worsening the situation. There is a need for novel therapeutics and drugs for treating and preventing hemophilia with inhibitor and diseases or disorders or conditions caused by FVII gene mutation or FVII protein deficiency.

### SUMMARY

To address the aforementioned problems, the present application provides small activating RNA (saRNA) molecules or oligonucleotide modulators comprising the same, for preventing and/or treating FVII-related diseases or conditions or disorders (for example blood coagulation diseases or disorders), for example those caused by the insufficient level of factor VII (FVII) mRNA or FVII protein, e.g., haploinsufficiency, or those with normal FVII levels but can be treated or prevented by increasing FVII levels, e.g., hemophilia (such as hemophilia with inhibitor), by activating/up-regulating FVII transcription and increasing the expression level of FVII protein via the RNA activation (RNAa) mechanism.

In particular, the inventors discovered that the functional saRNAs capable of activating/up-regulating the expression of FVII mRNA were not randomly distributed on the promoter but clustered in specific hotspot regions. Only some regions on the promotor of FVII gene are in favor of gene activation by saRNAs, for example, regions −557 to −379, −346 to −298, −271 to −91 and −96 to −1 upstream of the transcription start site of FVII gene. A hotspot region herein is defined by a nucleic acid region (such as in the promotor element upstream the TSS) on the target gene of the saRNAs, where the full length target sequences of a plurality of the functional saRNAs are located and enriched.

The inventors also discovered that optimal target sequences/sense strands of saRNAs within FVII promoter region include sequences having: (1) GC content between 40% and 70%; (2) no more than 5 consecutive identical nucleotides; (3) no more than 3 dinucleotide or trinucleotide repeats. As a beneficial consequence, a target sequence (e.g., an isolated nucleic acid sequence comprising the target sequence), upon interacting with the saRNA, can activate/up-regulate the expression of FVII mRNA by at least 10% as compared to a baseline level of FVII mRNA.

Based at least in part on the above surprising discoveries, the present disclosure features saRNAs, compositions, and pharmaceutical compositions for activating/up-regulating the expression of FVII mRNA, for example, by at least 10% as compared to baseline levels of FVII gene. Also provided herein are methods for preventing or treating FVII-related disease or condition or disorder, such as those induced by or associated with insufficient expression of factor VII (FVII), a FVII gene mutation, low functional FVII levels in blood in an individual and/or those with normal FVII level or function but can be prevented or treated by increasing endogenous FVII levels, such as hemophilia with inhibitor, comprising administering one or more of the saRNAs, compositions, and/or pharmaceutical compositions described herein or any combinations thereof.

In one aspect of the present application, a saRNA molecule or an oligonucleotide modulator comprising the same capable of activating/up-regulating expression of FVII gene in a cell is provided. The saRNA comprises an oligonucleotide sequence of 16 to 35 consecutive nucleotides in length, wherein the oligonucleotide sequence has at least 75%, at least 80%, at least 85%, at least 90%, at least 95%, at least 98%, at least 99%, at least 99.5% or 100% sequence homology or complementary to an equal length region of SEQ ID NO: 1437, and thereby activating or up-regulating the expression of the gene by at least 10% as compared to baseline expression of the FVII gene. In some embodiments, the equal length region of SEQ ID NO: 1437 is located in a region selected from the group consisting of: region −557 to −379; region −346 to −298; region −271 to −91; or region −96 to −1. In some embodiments, the equal length region of SEQ ID NO: 1437 is located in a region of SEQ ID NO: 1438, SEQ ID NO: 1439, SEQ ID NO: 1440, or SEQ ID NO: 1441.

In certain embodiments, the saRNA disclosed in the present application comprises a sense strand and an antisense strand, wherein the sense strand and the antisense strand each comprise complementary regions, wherein the complementary regions of the sense strand and the antisense strand form a double-stranded nucleic acid structure. In certain embodiments, the sense strand and the antisense strand disclosed in the present application have a complementarity of at least 75%. In certain embodiments, the sense strand and the antisense strand disclosed in the present application are located on two different nucleic acid strands. While in certain embodiments, the sense strand and the antisense strand disclosed in the present application are located on a contiguous nucleic acid strand, optionally a hairpin single-stranded nucleic acid molecule, wherein the complementary regions of the sense strand and the antisense strand form a double-stranded nucleic acid structure. In certain embodiments, one or both ends of the double-stranded nucleic acid structure can be blunt end(s). In certain embodiments, the sense strand and/or the antisense strand disclosed in the present application comprises a 3′ overhang ranging from 1 to 6 nucleotides in length, alternatively, from 2 to 3 nucleotides in length. In certain embodiments, at least one of the nucleotides of the overhang is a thymine deoxyribonucleotide. In certain embodiments, the overhang is a natural overhang. In certain embodiments, the sense strand and the antisense strand disclosed in the present application independently comprise about 16 to about 35, about 17 to about 30, about 18 to about 25, or about 19 to about 22 consecutive nucleotides.

In certain embodiments, the sense strand of the saRNA disclosed in the present application has at least 75% sequence homology to a nucleotide sequence selected from SEQ ID NOs: 287-858, and the antisense strand disclosed in the present application has at least 75% sequence homology to a nucleotide sequence selected from SEQ ID NOs: 859-1430. In certain embodiments, the sense strand disclosed in the present application comprises a nucleotide sequence selected from SEQ ID NOs: 287-858, and the antisense strand disclosed in the present application comprises a nucleotide sequence selected from SEQ ID NOs: 859-1430.

In certain embodiments, the sense strand of the saRNA disclosed in the present application has at least 75% sequence homology to a nucleotide sequence selected from SEQ ID NOs: 287-572, and the antisense strand disclosed in the present application has at least 75% sequence homology to a nucleotide sequence selected from SEQ ID NOs: 859-1430, wherein when the SEQ ID NO: of the nucleotide sequence of the sense strand is SEQ ID NO: n (wherein n is an integer selected from 287-572), the SEQ ID NO: of the reference nucleotide sequence of the antisense strand is SEQ ID NO: n+572 or n+858. For example, when the SEQ ID NO: of the nucleotide sequence of the sense strand is SEQ ID NO: 287, the SEQ ID NO: of the nucleotide sequence of the corresponding antisense strand is SEQ ID NO: 859 or SEQ ID NO: 1145.

In certain embodiments, the sense strand of the saRNA disclosed in the present application has at least 75% sequence homology to a nucleotide sequence selected from SEQ ID NOs: 573-858, and the antisense strand disclosed in the present application has at least 75% sequence homology to a nucleotide sequence selected from SEQ ID NOs: 859-1144, wherein when the SEQ ID NO: of the reference nucleotide sequence of the sense strand is SEQ ID NO: n′ (wherein n′ is an integer selected from 573-858), the SEQ ID NO: of the reference nucleotide sequence of the antisense strand is SEQ ID NO: n′+286.

In certain embodiments, the sense strand disclosed in the present application comprises a nucleotide sequence selected from SEQ ID NOs: 287-572, and the antisense strand disclosed in the present application comprises a nucleotide sequence selected from SEQ ID NOs: 859-1430, wherein when the SEQ ID NO: of the selected nucleotide sequence comprised in the sense strand is SEQ ID NO: n (wherein n is an integer selected from 287-572), the SEQ ID NO: of the selected nucleotide sequence comprised in the antisense strand is SEQ ID NO: n+572 or n+858. In certain embodiments, the sense strand disclosed in the present application comprises a nucleotide sequence selected from SEQ ID NOs: 573-858, and the antisense strand disclosed in the present application comprises a nucleotide sequence selected from SEQ ID NOs: 859-1144, wherein when the SEQ ID NO: of the selected nucleotide sequence comprised in the sense strand is SEQ ID NO: n′ (n′ is an integer selected from 573-858), the SEQ ID NO: of the selected nucleotide sequence comprised in the antisense strand is SEQ ID NO: n′+286.

In certain embodiments, the sense strand of the saRNA disclosed in the present application has at least 75% sequence homology to a nucleotide sequence selected from SEQ ID NOs: 1442, 1444, 1446, 1448, 1450, 1452, 1454, 1456, 1458, 1460, 1462, 1464, 1466, 1468, 1470, 1472, 1474 and 1476, and the antisense strand disclosed in the present application has at least 75% sequence homology to a nucleotide sequence selected from SEQ ID NOs: 1443, 1445, 1447, 1449, 1451, 1453, 1455, 1457, 1459, 1461, 1463, 1465, 1467, 1469, 1471, 1473 and 1475. In certain embodiments, the saRNA comprises the sense strand and the antisense strand to form duplexes as listed in Table 11.

In certain embodiments, the oligonucleotide sequence disclosed in the present application has at least 75% sequence homology or complementarity to a nucleotide sequence selected from SEQ ID NOs: 1-286. In certain embodiments, the sense strand of the oligonucleotide sequence disclosed in the present application has at least 75% sequence homology to a nucleotide sequence selected from SEQ ID NOs: 1-286. In certain embodiments, the antisense strand of the oligonucleotide sequence disclosed in the present application has at least 75% sequence complementarity to a nucleotide sequence selected from SEQ ID NOs: 1-286.

In certain embodiments, the sense strand of the saRNA disclosed in the present application has at least 75% sequence homology to a nucleotide sequence selected from SEQ ID NOs: 1-286, and the antisense strand disclosed in the present application has at least 75% sequence complementarity to the same reference nucleotide sequence selected from SEQ ID NOs: 1-286 for the sense strand.

In certain embodiments, at least one nucleotide of the saRNA disclosed in the present application is a chemically modified nucleotide. In certain embodiments, at least one nucleotide of the antisense and/or sense strand of the saRNA disclosed in the present application is chemically modified. In certain embodiments, the chemically modified nucleotide disclosed in the present application is a nucleotide with at least one the following modifications:
a) modification of a phosphodiester bond connecting nucleotides in the nucleotide sequence of the saRNA;
b) modification of 2′-OH of a ribose in the nucleotide sequence of the saRNA; and
c) modification of a base in the nucleotide sequence of the saRNA.

In certain embodiments, at least one nucleotide of the saRNA disclosed in the present application is a locked nucleic acid, an abasic nucleotide, a 2′-amino-modified nucleotide, a 2′-alkyl-modified nucleotide, a morpholino nucleotide, a phosphoramidate, or a non-natural base comprising nucleotide.

In certain embodiments, the chemical modification of the at least one chemically modified nucleotide disclosed in the present application is a phosphorothioate (PS) backbone modification.

In certain embodiments, the chemical modification of the at least one chemically modified nucleotide disclosed in the present application is a 2′ sugar modification selected from one or more of: 2′-fluoro-2′-deoxynucleoside (2′-F) modification, 2′-O-methyl (2′-O-Me), modification, and 2′-O-(2-methoxyethyl) (2′-O-MOE) modification.

In certain embodiments, the chemical modification of the at least one chemically modified nucleotide disclosed in the present application is an addition of a 5′-phosophate moiety at the 5′ end of the nucleotide sequence. In certain embodiments, the chemical modification of the at least one chemically modified nucleotide disclosed in the present application is an addition of a (E)-vinylphosphonate moiety or 5′-methyl cytosine moiety at the 5′ end of the sense strand and/or the antisense strand.

In certain embodiments, the disclosure provides oligonucleotide modulator wherein the sense strand and/or the antisense strand of the saRNA disclosed in the present application is conjugated to one or more conjugation moieties selected from a lipid, a fatty acid, a fluorophore, a ligand, a saccharide, a peptide, and an antibody.

In certain embodiments of the oligonucleotide modulator, the sense strand or the antisense strand of the saRNA disclosed in the present application is conjugated to one or more conjugation moieties selected from a cell-penetrating peptide, polyethylene glycol, an alkaloid, a tryptamine, a benzimidazole, a quinolone, an amino acid, a cholesterol, glucose, and N-acetylgalactosamine. In certain embodiments, the saRNA is conjugated to a lipid selected from C₄₋₃₀ fatty acid. In certain embodiments, the conjugation moiety is a lipid/fatty acid having a saturated or unsaturated, linear or branched C₁₆ carbon chain.

In certain embodiments of the oligonucleotide modulator, the saRNA is conjugated to two conjugation moieties, and the two conjugation moieties are a lipid and an N-acetylgalactosamine. In certain embodiments, the two conjugation moieties are independently selected from those derived from S9, tC2, tC2x6 and C5x5. In certain embodiments, the two conjugation moieties are: 1) one derived from S9, tC2 or tC2x6, and 2) one derived from C5x5.

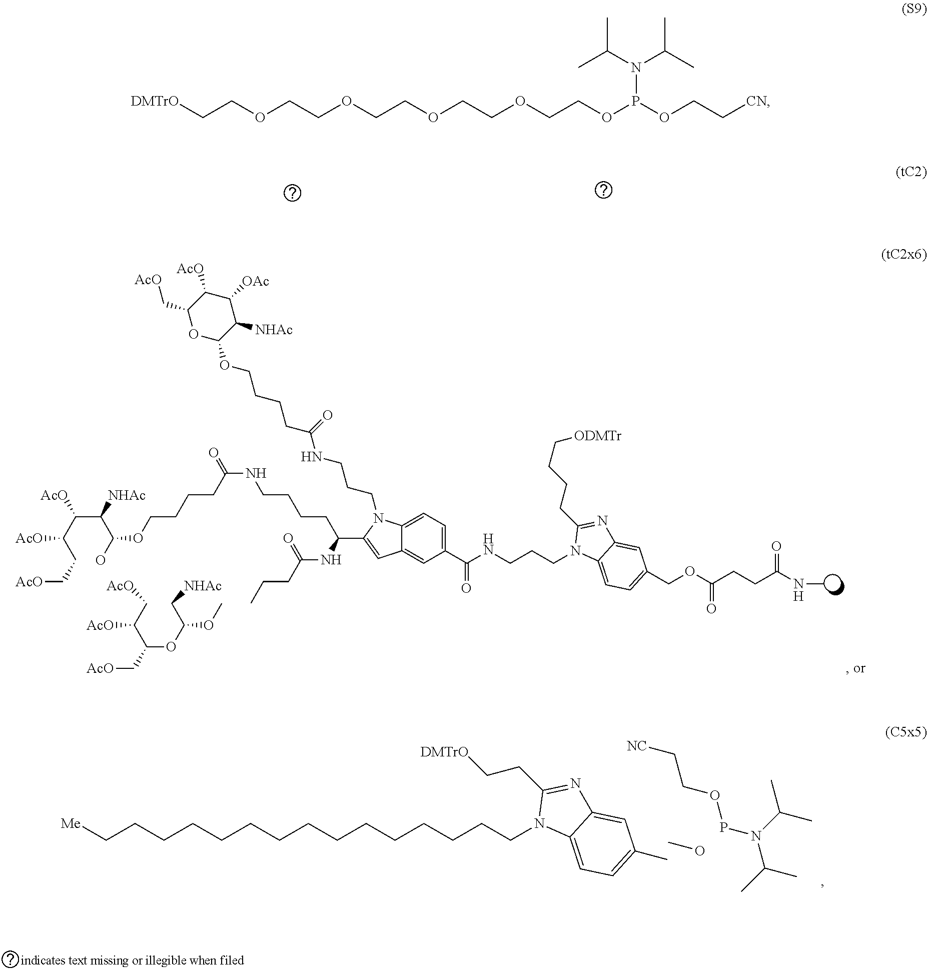

wherein [symbol] represents a support material.

In certain embodiments, the conjugation moieties conjugated to the saRNA are S9, tC2x6 and C5x5 as shown in the present application. In certain embodiments, tC2 or tC2x6 conjugates to the 3′ end of sense strand; C5x5 conjugates to the 5′ end of sense strand. The conjugation moieties can be synthesized via procedures known in the art, for example WO2024002046A1 is fully incorporated herein for synthetic process of tC2, tC2x6, C5x5; and S9 (HR-00214009, Anhui, China) is purchased from Wuhu Huaren Science and Technology Co., Ltd.

In another aspect of the present application, an isolated polynucleotide of saRNA is provided, wherein the isolated polynucleotide is a nucleotide sequence comprising 16 to 35 consecutive nucleotides of SEQ ID NO: 1437. Specifically, the isolated polynucleotide is a nucleic acid sequence selected from SEQ ID NOs: 1-286. In another aspect of the present application, methods of using the isolated polynucleotide of saRNA are provided.

In another aspect of the present invention, an oligonucleotide complex is provided, wherein the oligonucleotide complex comprises the antisense strand of the saRNA disclosed herein and the sense strand of the polynucleotide disclosed herein. In some embodiments, the oligonucleotide complex activates the expression of FVII gene (such as by at least 10%) as compared to baseline expression of FVII gene.

Another aspect of the present application provides a polynucleotide encoding the saRNA disclosed herein. In one embodiment, the saRNA disclosed herein is a small activating RNA (saRNA) molecule. In one embodiment, the polynucleotide is a DNA molecule. Another aspect of the present application provides a vector comprising the polynucleotide disclosed herein.

In another aspect of the present invention, a nucleic acid complex is provided, wherein the nucleic acid complex comprises the antisense strand of the saRNA disclosed herein and the sense strand of the polynucleotide disclosed herein. In some embodiments, the nucleic acid complex activates the expression of FVII gene (such as by at least 10%) as compared to baseline expression of the FVII gene.

Another aspect of the present application provides a cell comprising the saRNA disclosed herein, the polynucleotide encoding the saRNA disclosed herein, or the vector disclosed herein. In one embodiment, the cell is a mammalian cell, optionally a human cell. In some embodiments, the cell is a host cell. The aforementioned cell may be in vitro, such as a cell line or a cell strain, or may exist in a mammalian body, such as a human body.

Another aspect of the present application provides a composition, such as a pharmaceutical composition, comprising the aforementioned saRNA, the polynucleotide encoding the saRNA disclosed herein, the vector disclosed herein or the cell disclosed herein; and optionally, a pharmaceutically acceptable carrier. In some embodiments, the pharmaceutically acceptable carrier includes an aqueous carrier, a liposome, a high-molecular polymer or a polypeptide. In some embodiments, the pharmaceutically acceptable carrier is selected from an aqueous carrier, a liposome, a high-molecular polymer and a polypeptide. In some embodiments, the aqueous carrier may be, for example, RNase-free water or RNase-free buffer. In some embodiments, the composition may comprise 0.001-1600 nM (e.g., 0.001-1000 nM, 0.01-500 nM, 0.1-400 nM, 1-100 nM, 10-50 nM, 20-50 nM, 20-100 nM, 50-150 nM, 50-400 nM, 50-1000 nM or 400-1600 nM), or optionally 1-150 nM of the aforementioned saRNA or polynucleotide encoding the saRNA disclosed herein. In some embodiments, the composition may comprise 0.001-150 nM (e.g., 0.001-100 nM, 0.001-50 nM, 0.001-20 nM, 10-100 nM, 10-50 nM, 20-50 nM, 20-100 nM or 50-150 nM), or optionally 1~150 nM of the aforementioned saRNA or polynucleotide encoding the saRNA disclosed herein.

Another aspect of the present application relates to use of the aforementioned saRNA, polynucleotide encoding the saRNA disclosed herein or the vector or the composition comprising the aforementioned saRNA or polynucleotide disclosed herein in preparing a product for activating/up-regulating the expression of FVII gene in a cell.

The present application also relates to a method for activating/up-regulating the expression of FVII gene in a cell, wherein the method comprises administering the aforementioned saRNA, the polynucleotide disclosed herein or the vector or the composition comprising the aforementioned saRNA or polynucleotide disclosed herein to the cell. In the meantime, a method for increasing a level of FVII protein in a cell or a level of functional FVII protein in blood is also provided, comprising introducing a sufficient amount of the saRNA, the nucleic acid, or the composition disclosed herein into the cell.

The aforementioned saRNA, the polynucleotide disclosed herein or the composition comprising the aforementioned saRNA or polynucleotide disclosed herein may be directly introduced into a cell or may be produced in the cell after a nucleotide sequence encoding the saRNA is introduced into the cell. The cell is preferably a mammalian cell, more preferably a human cell. The aforementioned cell may be in vitro, such as a cell line or a cell strain, or may exist in a mammalian body, such as a human body. The human body is a patient suffering from FVII-related disease or symptom, such as those caused by or associated with a FVII gene mutation, low FVII level, insufficient blood levels of functional FVII protein in an individual, and/or those without FVII deficiency but can be prevented or treated by increased FVII levels, such as hemophilia (for example hemophilia with inhibitor), and the saRNA, the polynucleotide disclosed herein or the composition comprising the aforementioned saRNA or the polynucleotide disclosed herein is administered in a sufficient amount to treat the disease or symptom. Specifically, the symptoms caused by lack of FVII protein due to FVII gene mutation, insufficient expression of functional FVII protein, and/or hemophilia with inhibitor. In one embodiment, the disease or symptom is caused by insufficient expression of FVII protein, or FVII gene mutation, or insufficient blood levels of functional FVII protein. In some embodiments, the disease or symptom can be prevented and/or treated by increasing FVII level or function, such as hemophilia, e.g., hemophilia with inhibitor.

Another aspect of the present application relates to a method for preventing or treating FVII-related disease or condition or disorder. As used herein, the term “FVII-related disease(s) or condition(s) or disorder(s)” and “FVII associated disease(s) or condition(s) or disorder(s)” are interchangeable and refer to diseases or conditions or disorders that can be prevented or treated by increasing FVII level, for example those caused by or associated with insufficient expression of FVII protein, a FVII gene mutation, insufficient blood levels of functional FVII protein in an individual and/or those with normal FVII level or function but can still be prevented or treated by increasing FVII expression. FVII-related diseases or conditions or disorders may comprise but not limited to congenital FVII deficiency (Alexander's Disease), Acquired FVII deficiency (AFVIID), hemophilia (such as hemophilia A or B with or without inhibitor), Glanzmann's thrombasthenia (GT). The method of the present application comprises administering a therapeutically or prophylactically effective dose of the saRNA disclosed herein, the polynucleotide encoding the saRNA disclosed herein, the vector disclosed herein, or the composition comprising the saRNA disclosed to the individual. In certain embodiments, the disease or condition or disorder is hemophilia. The individual may be a mammal, such as a human. In one embodiment, the individual suffers from a symptom caused by or associated with insufficient expression of FVII protein, a FVII gene mutation, low functional FVII levels in blood, and/or hemophilia with inhibitor. In one embodiment, the disease or symptom is caused by insufficient expression of FVII protein, or FVII gene mutation, or insufficient blood levels of functional FVII protein, or hemophilia with inhibitor. In certain embodiments, the saRNA disclosed herein, the polynucleotide disclosed herein, the vector disclosed herein, or the composition disclosed herein is administrated to an individual by an administration route selected from one or more of: parenteral infusions or injection, oral administration, intranasal administration, inhaled administration, vaginal administration, and rectal administration. In certain embodiments, the administration route is selected from one or more of intrathecal, intramuscular, intravenous, intra-arterial, intraperitoneal, intravesical, intracerebroventricular, intravitreal and subcutaneous administrations. In certain embodiments, the method disclosed herein activates/up-regulates expression of FVII gene or FVII mRNA in the individual (such as by at least 10%, e.g., by at least 15%, by at least 20%, by at least 25%, by at least 30%, by at least 35%, by at least 40%, by at least 45%, by at least 50%, by at least 60%, by at least 70%, by at least 80%, by at least 90%, by at least 100%). In certain embodiments, the method disclosed herein increases a level of FVII protein in the individual (such as by at least 10%, e.g., by at least 15%, by at least 20%, by at least 25%, by at least 30%, by at least 35%, by at least 40%, by at least 45%, by at least 50%, by at least 60%, by at least 70%, by at least 80%, by at least 90%, by at least 100%).

Another aspect of the present application relates to use of the saRNA disclosed herein, the polynucleotide disclosed herein or the composition comprising the saRNA disclosed herein or the polynucleotide disclosed herein in preparing a medicament for preventing or treating FVII-related disorder or condition, such as those caused by or associated by insufficient blood levels of functional FVII protein, a FVII gene mutation, low functional FVII levels in blood, and/or hemophilia with inhibitor in an individual. The individual may be a mammal, such as a human. In one embodiment, the disease or disorder or condition may include, for example, hemophilia with inhibitor.

In addition, the present application further provides a kit for performing the method of prevention or treatment disclosed herein, wherein the kit comprises a) saRNA, b) instructions for use, and c) optionally, means for administering said saRNA to the individual. Specifically, a kit can be packed in a labeled package and the label on said package indicates that said molecule or composition can be used in preventing or treating FVII-related disease or disorder or condition, such as those induced by insufficient expression of factor VII (FVII), or against hemophilia with inhibitor. In some embodiments, a kit is provided by the present application for performing the method disclosed herein, wherein the kit comprises a) saRNA disclosed herein, and b) instructions for use. In certain embodiments, the instruction for use comprising means or methods for administering the saRNA disclosed herein to an individual.

Aspects of the present application include a kit comprising the saRNA disclosed herein, the polynucleotide disclosed herein, the vector disclosed herein, or the composition disclosed herein in a labeled package and the label on package indicates that the saRNA, the polynucleotide, the vector or the composition can be used in preventing or treating FVII-related disease or condition or disorder, such as those induced by insufficient expression of factor VII (FVII), or hemophilia with inhibitor.

Further to provide by the present application is a kit for detecting FVII protein, or FVII regulated protein in blood, or in a cell disclosed herein having been transfected with the saRNA aforementioned, or the nucleic acid aforementioned, or the composition aforementioned.

The oligonucleotide modulators activating/up-regulating the expression of FVII gene provided herein (such as a saRNA molecule) can efficiently and specifically up-regulate the expression of FVII gene and increase the expression level of FVII mRNA with low toxic and adverse effects, and can be used in preparing a drug for preventing or treating FVII-related diseases or condition or disorders, such as disorders associated with insufficient expression of FVII protein and diseases or conditions caused by a FVII gene mutation or such conditions as hemophilia with inhibitor.

### BRIEF DESCRIPTION OF THE DRAWINGS

FIG. 1 shows changes in expression level of human FVII mRNA mediated by saRNAs in human hepatocarcinoma cell line (Huh-7). 858 human FVII promoter-targeting saRNAs were individually transfected into Huh-7 cells at 25 nM for 72 hours. Strand composition and sequence of each saRNA duplex including cognate target site in the human FVII promoter is listed in Table 1.1. Cells were transfected in the absence of an oligonucleotide as Mock treatment (not shown). dsCon2 duplex served as a non-targeting control (not shown). RD-13516 was a duplex siRNA targeting FVII mRNA and transfected as a silencing double-stranded RNA (siRNA) control (not shown). FVII mRNA levels were quantified by one-step RT-qPCR using a gene specific primer set (Table 3) in each of the PCR reactions. Geometric means of the mRNA levels of PGK1 and SDHA were used as an internal reference. The value (y-axis, log 2 fold change) shows the relative fold changes on FVII mRNA expression levels by saRNAs relative to Mock treatment after normalized to PGK1 and SDHA (mean±SEM of two replicated transfection wells). saRNAs are sorted on x-axis by their activity in inducing FVII mRNA expression (log 2 fold change) in a descending order.

FIG. 2 shows the activity of saRNAs sorted by their target locations and saRNA hotspot regions on human FVII promoter in Huh-7 cells. 858 human FVII promoter-targeting saRNAs were individually transfected into Huh-7 cells at 25 nM for 72 hours. Cells were transfected in the absence of an oligonucleotide as Mock treatment (not shown). dsCon2 duplex served as a non-targeting control (not shown). RD-13516 was a duplex siRNA targeting FVII mRNA and transfected as a silencing siRNA control (not shown). mRNA levels of FVII were quantified by one-step RT-qPCR using a gene specific primer set in individual PCR reactions. Geometric means of the mRNA levels of PGK1 and SDHA were used as an internal reference. The value (y-axis, log 2 fold change) shows the relative fold changes on FVII mRNA expression levels by each of the saRNAs relative to Mock treatment after normalized to PGK1 and SDHA (mean±SEM of two replicated transfection wells). saRNAs are sorted on x-axis by their target locations on the promoter −558 to −1 upstream of the FVII transcription start site (TSS). Four saRNA hotspot regions with enriched targets for functional saRNAs (activating dsRNAs) were marked as H1 to H4 in rectangular dotted boxes. The numbers above the boxes indicate the boundaries of the hotspot regions relative to the FVII TSS (site 0 on x-axis) which span the very 5′ end of the first functional saRNA's target and the very 3′ end of the last saRNA's target within each hotspot region.

FIG. 3 shows saRNA induction of FVII mRNA level in Huh-7 cells. Based on the screening results for FVII mRNA induction, top 47 functional FVII saRNAs (i.e., saRNAs with the highest relative fold changes on FVII mRNA expression levels determined in Examples 1 and 2) were selected and transfected into Huh-7 cells at 7 indicated concentrations (i.e., 0.1, 0.39, 1.56, 6.25, 25, 100 and 400 nM) for 72 hours. Cells were transfected in the absence of an oligonucleotide as Mock treatment (not shown). dsCon2 duplex served as a non-targeting control (not shown). RD-13516 was a duplex siRNA targeting FVII mRNA and transfected as a silencing siRNA control (not shown). FVII mRNA levels were quantified by one step RT-qPCR using a gene specific primer set in individual PCR reactions. Geometric means of the mRNA levels of PGK1 and SDHA were used as an internal reference. The values (y-axis) are presented FVII mRNA expression levels of 7-point dose response by each of saRNA relative to Mock treatment after normalized to the reference of PGK1 and SDHA (mean±SEM of two replicated transfection wells).

FIG. 4 shows the activating effect of saRNAs on the expression of FVII protein in HepG2 cells. 19 indicated saRNAs (i.e., DS20-027A, DS20-055A, DS20-029S, DS20-124S, DS20-086A, DS20-207B, DS20-151B, DS20-228B, DS20-069A, DS20-177A, DS20-207S, DS20-272A, DS20-252A, DS20-156S, DS20-205S, DS20-241A, DS20-082A, DS20-009A and DS20-188B) were individually transfected into HepG2 cells at 25 nM for 4 days. Cells were transfected in the absence of an oligonucleotide as Mock treatment. dsCon2 duplex served as a non-targeting control. FVII protein levels were detected by western blotting using an antibody against human FVII protein. An antibody against β-Tubulin was also blotted to serve as a control for protein loading. The values (y-axis) are presented as FVII protein expression level relative to Mock treatment after normalized to β-Tubulin (mean±SEM of two replicated transfection wells).

FIGS. 5A-5B show the activating activity of chemically modified saRNAs (CM-saRNAs) on the expression of FVII mRNA in HepG2 and Huh-7 cells. 10 indicated FVII CM-saRNAs (i.e., RD-16036, RD-16037, RD-16038, RD-16027, RD-16028, RD-16041, RD-16052, RD-16055, RD-16134 and RD-16035) were transfected into HepG2 cells at 10 nM for 3 days. 11 indicated FVII CM-saRNAs (i.e., RD-16036, RD-16037, RD-16038, RD-16027, RD-16028, RD-16041, RD-16052, RD-16055, RD-16134, RD-16044 and RD-16035) were transfected into Huh-7 cells at 10 nM for 3 days. Cells were transfected in the absence of an oligonucleotide as Mock treatment. dsCon2M6v duplex served as a non-targeting control. RD-15120 was a chemically modified siRNA for FVII gene and transfected as a silencing siRNA control. FVII mRNA levels were quantified by two step RT-qPCR using a gene specific primer set in individual PCR reactions. Geometric means of the mRNA levels of PGK1 and SDHA were used as an internal reference. FIG. 5A shows the FVII mRNA levels in HepG2 cells. FIG. 5B shows the FVII mRNA levels in Huh-7 cells. The values (y-axis) are presented FVII mRNA levels by each of saRNA relative to Mock treatment after normalized to the reference of PGK1 and SDHA (mean±SEM of four replicated transfection wells).

FIGS. 6A-6B show the activating effect of CM-saRNAs on the expression of FVII protein in HepG2 and Huh-7 cells. 7 indicated FVII CM-saRNAs (i.e., RD-16012, RD-16036, RD-16013, RD-16017, RD-16029, RD-16041 and RD-16120) were transfected into HepG2 cells at 25 nM for 4 days. 7 indicated FVII CM-saRNAs (i.e., RD-16012, RD-16024, RD-16036, RD-16017, RD-16029, RD-16041 and RD-16120) were transfected into Huh-7 cells at 25 nM for 4 days. Cells were transfected in the absence of an oligonucleotide as Mock treatment. dsCon2M3v duplex served as a non-targeting control. RD-15120 was a chemically modified siRNA for FVII gene and transfected as a silencing siRNA control. FVII protein levels were detected by western blotting using an antibody against human FVII protein. An antibody against β-Tubulin was also blotted to serve as a control for protein loading. FIG. 6A shows the FVII protein levels in HepG2 cells. FIG. 6B shows the FVII protein levels in Huh-7 cells. The values (y-axis) are presented as FVII protein expression level relative to Mock treatment after normalized to β-Tubulin (mean±SEM of two replicated transfection wells).

### DETAILED DESCRIPTION

Double-stranded RNAs (dsRNAs) targeting gene regulatory sequences, including promoters, have been shown to up-regulate target genes in a sequence-targeting manner at the transcriptional level via a mechanism known as RNA activation (RNAa) (Li, L. C., et al. Small dsRNAs induce transcriptional activation in human cells. PNAS (2006)). Such dsRNAs are termed small activating RNAs (saRNAs).

Embodiments of the present disclosure are based in part on the surprising discovery that an oligonucleotide modulator (for example, those comprising an saRNA, also referred to as “FVII gene saRNA” herein) can activate or up-regulate the expression of a FVII gene in a cell. The increase in production of functional FVII gene mRNA following administration with a saRNA of the present application can achieve a significant increase or up-regulation in the level of FVII mRNA and FVII protein.

In particular, the inventors discovered that the functional saRNAs capable of activating/up-regulating the expression of FVII mRNA were not randomly distributed on the promoter but clustered in certain specific hotspot regions. Only some regions on the promotor of FVII gene are in favor of gene activation by saRNAs, for example, regions −557 to −379 (H1), −346 to −298 (H2), −271 to −91 (H3) and −96 to −1 (H4) upstream of the transcription start site of FVII gene. These specific promoter regions (referred to as “hotspot” or “hotspot region” herein) identified by the present application are optionally at least 25 nt, at least 27 nt, at least 30 nt, at least 35 nt, at least 40 nt, at least 45 nt, at least 48 nt, or at least 49 nt in length; have a length ranging from about 25 to 200 nt, about 30 to 190 nt, about 40 to 185, or about 49 to about 181 nt; or have a length in a narrower numerical range or a particular numerical point (such as 181 nt, 179 nt, 96 nt, 49 nt, 27 nt, 25 nt) that falls within the above broader numerical ranges.

The inventors also discovered that optimal target sequences/sense strand of a saRNA within the FVII promoter region include sequences having criteria of: (1) GC content between 40% and 70%; (2) no more than 5 consecutive identical nucleotides; (3) no more than 3 dinucleotide or trinucleotide repeats. As a beneficial consequence of the criteria, a target sequence (e.g., an isolated nucleic acid sequence comprising the target sequence), upon interacting with the saRNA, can activate/up-regulate the expression of FVII mRNA by at least 10% or 1.1 fold as compared to a baseline level of FVII mRNA.

In some embodiments, a hotspot region is a nucleic acid region on the target gene of the saRNAs spanning the very 5′ end of the first saRNA's target and the very 3′ end of the last saRNA's target within each hotspot wherein at least 20% (such as at least 30%, at least 40%, at least 45%, about 50%) of the saRNAs designed according to the criteria (1), (2), (3), and (4) listed above targeting the region are turned out to be functional, i.e., can activate/up-regulate the mRNA expression of the target gene by 1.1-fold or more as compared to the baseline level of the mRNA expression. In some embodiments, at least 20%, about 22%, at least 30%, about 35%, about 40%, or over 50% of the saRNAs designed are functional, i.e., can activate/up-regulate the mRNA level or protein expression of the target gene by 1.1-fold or more.

Based at least in part on these discoveries, the present disclosure features saRNAs, compositions, and pharmaceutical compositions for activating/up-regulating the expression of FVII mRNA (such as by at least 10%) as compared to baseline levels of FVII mRNA. Also provided herein are methods for preventing or treating FVII-related disease or condition or disorder, such as the one induced by insufficient expression of factor VII (FVII), a FVII gene mutation, low functional FVII levels in blood in an individual and/or hemophilia with inhibitor comprising administering any of the saRNA, compositions, and/or pharmaceutical compositions described herein.

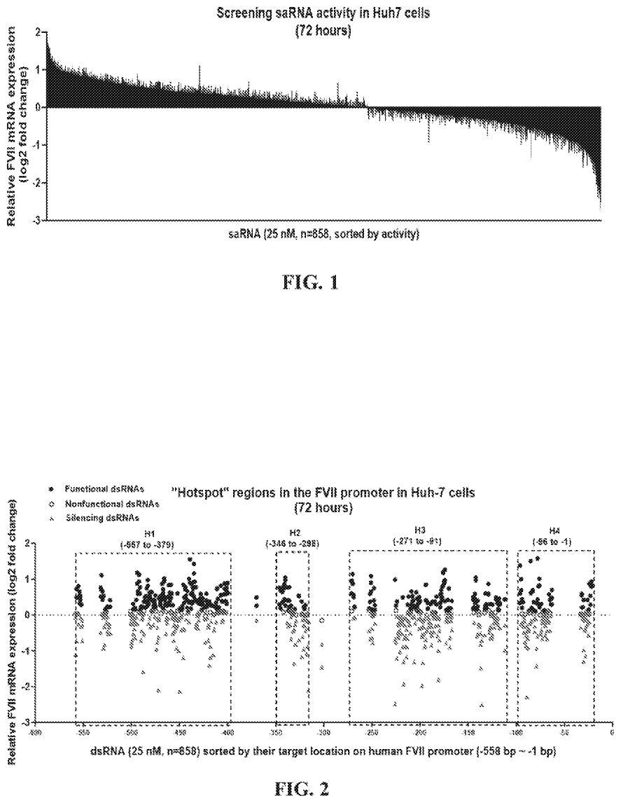

Embodiments of the present disclosure are also based in part on the surprising discovery that the target sequences of the saRNAs capable of activating or up-regulating the expression of FVII gene in a cell are clustered in particular FVII gene promoter regions, as shown in FIG. 2. The present inventors identified these clusters of FVII gene promoter regions that were considered “hotspot” promoter regions that enrich target sites for the functional saRNAs developed (see e.g., Table 8). For example, the 4 hotspot regions of the human FVII promoter located in regions −557 to −379 (H1), −346 to −298 (H2), −271 to −91 (H3) and −96 to −1 (H4) from the TSS of the promoter were detected and were found to be optimal target sites for saRNAs in activating FVII gene expression by the RNA activation mechanism.

This saRNA-FVII mRNA-FVII protein pathway can provide an alternative therapeutic method different from the current treatment of FVII-deficiency-related disorders and other disorders including hemophilia with inhibitor.

### Definitions

In the present application, the related terms are defined as follows:

Every numerical range given throughout this specification will include every narrower numerical range that falls within such broader numerical range, as if such narrower numerical ranges were all expressly written herein.

The transitional terms/phrases (and any grammatical variations thereof) “comprising”, “comprises”, “comprise”, include the phrases “consisting essentially of”, “consists essentially of”, “consisting”, and “consists” and can be interchanged throughout the application. The open term “comprise” also includes a closed term “consisting of” as one option. As used herein, the terms “include,” “have” and “comprise” are used synonymously, which terms and variants thereof are intended to be construed as non-limiting.

The term “complementary” as used herein refers to the capability of forming base pairs between two oligonucleotide strands. The base pairs are generally formed through hydrogen bonds between nucleotides in the antiparallel oligonucleotide strands. The bases of the complementary oligonucleotide strands can be paired in the Watson-Crick manner (such as A to T, A to U, and C to G) or in any other manner allowing the formation of a duplex (such as Hoogsteen or reverse Hoogsteen base pairing).

Complementarity includes complete complementarity and incomplete complementarity. “Complete complementarity” or “100% complementarity” means that each nucleotide from the first oligonucleotide strand can form a hydrogen bond with a nucleotide at a corresponding position in the second oligonucleotide strand in the double-stranded region of the double-stranded oligonucleotide molecule, with no base pair being “mispaired”. “Incomplete complementarity” means that not all the nucleotide units of the two strands are bound with each other by hydrogen bonds. For example, for two oligonucleotide strands each of 20 nucleotides in length in the double stranded region, if only two base pairs in this double-stranded region can be formed through hydrogen bonds, the oligonucleotide strands have a complementarity of 10%. In the same example, if 18 base pairs in this double-stranded region can be formed through hydrogen bonds, the oligonucleotide strands have a complementarity of 90%. Substantial complementarity refers to at least about 75%, about 79%, about 80%, about 85%, about 90%, about 95% or 99% complementarity.

The term “oligonucleotide” or “polynucleotide” can be used interchangeably, and refers to polymers of nucleotides, and includes, but is not limited to, single-stranded or double-stranded nucleic acid molecules of DNA, RNA, or DNA/RNA hybrid, oligonucleotide strands containing regularly and irregularly alternating deoxyribosyl portions and ribosyl portions, as well as modified and naturally or unnaturally existing frameworks for such oligonucleotides. The oligonucleotide for activating target gene transcription described herein can be or may comprise a small activating nucleic acid molecule (saRNA).

The terms “oligonucleotide strand”, “strand” and “oligonucleotide sequence” as used herein can be used interchangeably, referring to a generic term for short nucleotide sequences having less than 35 bases (including nucleotides in deoxyribonucleic acid (DNA) or ribonucleic acid (RNA)). In a non-limiting example, the length of a strand can be any length from 16 to 35 nucleotides.

The term “target gene” as used herein can refer to nucleic acid sequences, transgenes, viral or bacterial sequences, chromosomes or extrachromosomal genes that are naturally present in organisms, and/or can be transiently or stably transfected or incorporated into cells and/or chromatins thereof. The target gene can be a protein-coding gene or a non-protein-coding gene (such as a microRNA gene and a long non-coding RNA gene). The target gene generally contains a promoter sequence, and the positive regulation for the target gene can be achieved by designing a saRNA having sequence identity (also called homology) to the promoter sequence, characterized as the up-regulation of expression of the target gene. “Target sequence” or “target site” used interchangeably refers to a sequence fragment in the sequence of a target gene, such as, a target gene promoter, which is homologous or complementary with a sense strand or an antisense strand of a saRNA. The target gene can also include one or more regulatory elements where one or more saRNA are designed to have sequence identity to a regulatory element. Non-limiting examples of one or more regulatory elements include: a promoter, an enhancer, a silencer, an insulator, a TATA box, a GC box, a CAAT box, a transcriptional start site, a DNA binding motif of a transcription factor or other protein that regulates transcription, and a 5′ untranslated region.

As used herein, the terms “sense strand” of a saRNA in the saRNA duplex refers to the strand having sequence homology or sequence identity with a fragment of the coding strand of the sequence of a target gene.

As used herein, the terms “antisense strand” of a saRNA in the saRNA duplex refers to the strand having sequence complementary with the sense strand. Said antisense strand may interact with a target sequence to active or up-regulate gene expression, said target sequence may be a fragment of the coding strand of the sequence of a target gene.

The term “coding strand” as used herein refers to a DNA strand in the target gene which cannot be used for transcription, and the nucleotide sequence of this strand is the same as that of an RNA produced from transcription (in the RNA, T in DNA is replaced by U). The coding strand of the double-stranded DNA sequence of the target gene promoter described herein refers to a promoter sequence on the same DNA strand as the DNA coding strand of the target gene.

The term “template strand” as used herein refers to the other strand complementary with the coding strand in the double-stranded DNA of the target gene, i.e., the strand that, as a template, can be transcribed into RNA, and this strand is complementary with the transcribed RNA (A to U and G to C). In the process of transcription, RNA polymerase binds to the template strand, moves along the 3′→5′ direction of the template strand, and catalyzes the synthesis of the RNA along the 5′→3′ direction. The template strand of the double-stranded DNA sequence of the target gene promoter described herein refers to a promoter sequence on the same DNA strand as the DNA template strand of the target gene.

As used herein, the term “LNA” refers to a locked nucleic acid in which the 2′-oxygen and 4′-carbon atoms are joined by an extra bridge. As used herein, the term “BNA” refers to a 2′-O and 4′-aminoethylene bridged nucleic acid that can contain a five-membered or six-membered bridged structure with an N—O linkage. As used herein, the term “PNA” refers to a nucleic acid mimic with a pseudopeptide backbone composed of N-(2-aminoethyl) glycine units with the nucleobases attached to the glycine nitrogen via carbonyl methylene linkers.

The term “promoter” as used herein refers to a sequence which is spatially associated with a protein-coding or RNA-coding nucleic acid sequence and plays a regulatory role for the transcription of the protein-coding or RNA-coding nucleic acid sequence. Generally, a eukaryotic gene promoter contains 100 to 5000 base pairs, although this length range is not intended to limit the term “promoter” as used herein. Although the promoter sequence is generally located at the 5′ terminus of a protein-coding or RNA-coding sequence, it may also exist in exon and intron sequences.

The term “transcription start site (TSS)” as used herein refers to a nucleotide marking the transcription start on the template strand of a gene. The transcription start site can appear on the template strand of the promoter region. In particular, a “transcription starting site (TSS)” may refer to a location where the transcription start at the 5′-end of an FVII gene sequence, which is a nucleotide that marks the initiation of transcription on the template strand of a gene, and corresponds to the first nucleotide on the RNA molecule transcribed from the gene. Different variants of a gene may have different TSS, but with identical upstream sequences. Conventional means can be used to find the TSS in a gene, such as cap analysis of gene expression (CAGE), oligo-capping and robust analysis of 5′-transcript ends (5′-RACE).

In some embodiments, the target site is selected based at least in part on a gene sequence. In some embodiments, the target site is selected based at least in part on a sequence close to a transcription starting site (TSS) of the gene. In some embodiments, coding strand sequence from the promoter of human FVII gene can be retrieved from the UCSC genome database, for example, SEQ ID NO: 1437 (as shown in Table 6) consisting of 600 nucleotides ranging from position −1 bp to −600 bp relative to the transcription start site (TSS).

The term “identity” or “homology” as used herein means that one oligonucleotide strand (sense or antisense strand) of a saRNA has sequence similarity with a coding strand or template strand in a region of a target gene. As used herein, the “identity” or “homology” may be at least about 75%, about 79%, about 80%, about 85%, about 90%, about 95% or 99%. In some embodiments, the saRNA has 1, 2, 3, 4, 5, 6, 7, 8, 9, 10 residues that are different from a reference sequence. To determine the percent identity of two nucleic acid sequences, the sequences are aligned for optimal comparison purposes (e.g., gaps can be introduced in one or both of a first and a second nucleic acid sequence for optimal alignment and non-homologous sequences can be disregarded for comparison purposes). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm, such as using the Needleman and Wunsch ((1970) J. Mol. Biol. 48:444-453) algorithm which has been incorporated into the GAP program in the GCG software package (available at www.gcg.com). The percent identity between two nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4. It is understood that the molecules described herein may have additional conservative or non-essential nucleic acid substitutions, which do not have a substantial effect on their functions.

The term “equal length portion” refers to a portion of a sequence that is compared with an object sequence (e.g., a continuous oligonucleotide sequence from the saRNA) and has equal length (equal number of bases) to the object sequence.

The term “sequence specific mode” as used herein means a binding or hybridization way of two nucleic acid fragments according to their nucleotide sequence, e.g., a Watson-Crick manner (such as A to T, A to U, and C to G) or any other manner allowing the formation of a duplex (such as Hoogsteen or reverse Hoogsteen base pairing).

The term “overhang” as used herein refers to non-base-paired nucleotides at the terminus (5′ or 3′) of an oligonucleotide strand, which is formed by one strand extending out of the other strand in a double-stranded oligonucleotide. A single-stranded region extending out of the 3′ terminus and/or 5′ terminus of a duplex is referred to as an overhang.

The term “natural overhang” as used herein refers to an overhang which consists of one or more nucleotides identical to or complementary to the corresponding position on the target sequence. A natural overhang on a sense strand consists of one or more nucleotides identical to the corresponding position on the DNA target. A natural overhang on a sense strand consists of one or more nucleotides identical to the corresponding position on the DNA target. A natural overhang on an antisense strand consists of one or more nucleotides complementary to the corresponding position on the DNA target.

As used herein, the term “isolated,” refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotide could be part of a vector and/or such polynucleotide or polypeptide could be part of a composition, and still be isolated in that such vector or composition is not part of the environment in which it is found in nature. An isolated molecule may be obtained, for example, by extraction from a natural source, by expression of a recombinant nucleic acid, or by chemically synthesizing the molecule. For example, the term “isolated RNA” refers to RNA molecules which are substantially free of other cellular material, or culture medium when produced by recombinant techniques, or substantially free of chemical precursors or other chemicals when chemically synthesized. In some embodiments, the materials of the present application, such as the polynucleotides, oligonucleotides and/or saRNAs of the present application, are isolated.

As used herein, the terms “gene activation” or “activating gene expression” and “gene up-regulation” or “up-regulating gene expression” can be used interchangeably, and mean an increase in transcription, translation, expression or activity of a certain nucleic acid as determined by measuring the transcriptional level, mRNA level, protein level, enzymatic activity, methylation state, chromatin state or configuration, translation level or the activity or state in a cell or biological system of a gene. These activities or states can be determined directly or indirectly. In addition, “gene activation”, “activating gene expression”, “gene up-regulation” or “up-regulating gene expression” refers to an increase in activity associated with a nucleic acid sequence, regardless of the mechanism of such activation. For example, gene activation occurs at the transcriptional level to increase transcription into RNA and the RNA is translated into a protein, thereby increasing the expression of the protein.

As used herein, the terms “small activating RNA”, “saRNA”, and “small activating nucleic acid molecule” can be used interchangeably, and refer to a nucleic acid molecule that can up-regulate target gene expression and can be composed of a first nucleic acid fragment (sense strand) containing a nucleotide sequence having high sequence identity to the non-coding nucleic acid sequence (e.g., a promoter or an enhancer) of a target gene and a second nucleic acid fragment (antisense strand) containing a nucleotide sequence complementary with the first nucleic acid fragment, wherein the first nucleic acid fragment and the second nucleic acid fragment form a duplex. The saRNA can also be comprised of a synthesized or vector-expressed single-stranded RNA molecule that can form a hairpin structure by two complementary regions within the molecule, wherein the first region contains a nucleotide sequence having sequence identity to the target sequence of a promoter of a gene, and the second region contains a nucleotide sequence which is complementary with the first region. The length of the duplex region of the saRNA is typically about 15 to about 35, about 16 to about 32, about 17 to about 30, about 18 to about 28, about 19 to about 26, about 20 to about 24, and about 21 to about 22 base pairs, and typically about 15, about 16, about 17, about 18, about 19, about 20, about 21, about 22 or about 23 base pairs. In addition, the terms “saRNA”, “small activating RNA”, and “small activating nucleic acid molecule” also contain nucleic acids other than the ribonucleotide, including, but not limited to, modified nucleotides or analogues.

The terms “oligonucleotide modulator” refer to an oligonucleotide-containing substance which at least comprises or consists of one or more saRNA of the invention and has the activity of modulating target gene expression or enhance the effect of the saRNA, and may further comprise other oligonucleotide moieties/components (such as ASO) or non-oligonucleotide moieties/components conjugated, combined or mixed with the saRNA(s). In certain embodiments, the oligonucleotide modulator comprises an RNA (such as the saRNA of the invention), a DNA, a BNA, an LNA, or a peptide nucleic acid (PNA).

As used herein, the terms “hotspot region” and “hotspot” can be used interchangeably, and herein is defined by a nucleic acid region (such as in the promotor upstream the TSS of the target gene) on the target gene of the saRNAs, where full length targets of functional saRNAs are enriched and which span the very 5′ end of the first saRNA's target and the 3′ end of the last saRNA's target within each hotspot. “Hotspot region” is a gene promoter region of at least 25 bp (such as at least 49 bp) in length where target sequences of functional saRNAs are enriched, e.g., at least 20%, e.g., about 22%, about 30%, about 40%, about 50%, about 60%, about 70%, about 80%, about 90% or more than 90% of the saRNAs designed to target this region are “functional”, i.e., can induce a 1.1-fold or more change in the mRNA or protein expression of the target gene, provided that the saRNAs are designed according to the following criteria: (1) GC content between 40% and 70%; (2) no more than 5 consecutive identical nucleotides; (3) no more than 3 dinucleotide or trinucleotide repeats.

As used herein, the term “functional saRNA” refers to a saRNA which activates the expression of its intended target gene (such as by at least 10% or at least 1.1 fold). The term “non-functional saRNA” refers to a saRNA which modulates both mRNA level and protein expression of FVII gene (such as by less than 10% or less than 1.1 fold).

As used herein, the term “a target site” and “an oligonucleotide” can be used interchangeably, and herein means a target site which a saRNA has complementarity or hybridizes to. For example, an oligonucleotide of a target site can include a nucleic acid sequence which a region of saRNAs have complementarity or hybridize to. As used herein, the term “a polynucleotide” in the context of saRNA means a polynucleotide which encodes a saRNA, for example a DNA.

As used herein, the term “synthesis” refers to a method for synthesis of an oligonucleotide, including any method allowing RNA synthesis, such as chemical synthesis, in vitro transcription, and/or vector-based expression.

As used herein, the term “support material” refers to a solid phase starting material held between filters, in columns that enable all reagents and solvents to pass through freely using an automated oligonucleotide synthesizer, and optionally, generate 3′ or 5′ end conjugated oligonucleotide. A support material can be selected from the group consisting of control pore glass (CPG), silica, silica gel, glass, ceramic, polymer, cellulose, and combinations thereof.

As used herein, the upper cased “FVII” or “FVII gene” refers to a human gene. As used herein, the term “FVII mRNA” refers to a message RNA (mRNA) generated from the expression of FVII gene, or the transcription of FVII gene.

As used herein, the term “FVII” and “FVII protein” can be used interchangeably, and refers to a protein generated from the expression of FVII gene, or translation of the FVII mRNA.

As used herein, the term “baseline expression of FVII gene” refers to the expression of FVII gene of a parallel reference (such as a cell or an individual) without or before the treatment of the saRNA.

saRNA

In the present application, expression of the FVII gene is up-regulated by RNA activation, and a related disease is treated by increasing the expression level of FVII protein. As the FVII gene encodes the FVII protein, an increase in FVII mRNA expression results in an increase in expression of the FVII protein, thereby preventing or treating the disease (e.g., hemophilia with inhibitor). Therefore, the FVII gene, in some cases, is a target gene in the present application.

Aspects of the present application include a saRNA (or an oligonucleotide modulator comprising the saRNA) comprising an oligonucleotide sequence having a length ranging from 16 to 35 consecutive nucleotides, wherein the continuous oligonucleotide sequence has at least 75%, or at least 80%, or at least 85%, or at least 90% sequence homology or complementary to an equal length portion of SEQ ID NO: 1437, and wherein the saRNA activates/up-regulates the expression of FVII gene (such as by at least 10%) as compared to baseline expression of the FVII gene.

In some embodiments, the equal length portion of SEQ ID NO: 1437 disclosed herein is located in the region −557 to −379, region −346 to −298, region −271 to −91, or region −96 to −1 upstream of the transcription start site of the FVII gene. In some embodiments, the equal length region of SEQ ID NO: 1437 is located in a region of SEQ ID NO: 1438, SEQ ID NO: 1439, SEQ ID NO: 1440, or SEQ ID NO: 1441.

In some embodiments, the continuous oligonucleotide sequence of the saRNA has five or less, i.e., 5, 4, 3, 2, 1, or 0 nucleotide differences or mismatches relative to the equal length portion of SEQ ID NO: 1437. In some embodiments, the differences or mismatches are located in the middle or 3′ terminus of the oligonucleotide sequence of the saRNA. Methods and principles of saRNA molecule design are well known to those skilled in the art and are described in detail in, for example, Place et. al., Molecular Therapy—Nucleic Acids (2012) 1, e15; and Li et. al., PNAS, 2006, vol. 103, no. 46, 17337-17342, which are herein incorporated by reference in their entireties.

In some embodiments, the saRNA disclosed herein comprises a sense strand and an antisense strand. The sense strand and the antisense strand each comprise complementary regions capable of forming a double-stranded nucleic acid structure that activates the expression of the FVII gene in a cell via the RNAa mechanism. The RNAa mechanism (also known as RNA activation) used herein refers to a mechanism that a double-stranded nucleic acid structure is capable of up-regulating target genes in a sequence-specific manner at the transcriptional level.

Aspects of the present invention also include a small activating RNA (saRNA) comprising a sense strand and an antisense strand. The sense strand and the antisense strand each comprises a complementary region.

The sense strand and the antisense strand of the saRNA can exist either on two different nucleic acid strands or on one nucleic acid strand (e.g., a contiguous nucleic acid sequence). When the sense strand and the antisense strand are located on two different strands, one or both ends of the saRNA can be blunt end(s), or at least one strand of the saRNA has a 3′ or 5′ overhang of 1 to 6 nucleotides in length, such as overhang(s) of 1, 2, 3, 4, 5 or 6 nucleotides in length. In some cases, both strands have a 3′ or 5′ overhang of 1 to 6, e.g., 2 or 3 nucleotides in length. The nucleotide of the overhang is, in some cases thymine deoxyribonucleotide (dT). In some cases, the overhang is a natural overhang. When the sense strand and the antisense strand are located on one nucleic acid strand, in some cases, the saRNA is a hairpin single-stranded nucleic acid molecule, where the complementary regions of the sense strand and the antisense strand form a double-stranded nucleic acid structure with each other.

In some embodiments, the saRNA is a duplex comprising of a sense strand and an antisense strand complementary to each other resulting in blunt-end structures at both termini. In some embodiments, the saRNA is a duplex comprising of a sense strand and an antisense strand complementary to each other resulting in which the antisense strand has an overhang of 1-6 nucleotides in length on the 3′-terminus of the antisense strand. In some embodiments, the saRNA is a duplex comprising of a sense strand and an antisense strand complementary to each other resulting in which the sense strand has an overhang of 1-6 nucleotides in length on the 3′-terminus of the sense strand.

In the aforementioned saRNA, in some embodiments, the sense strand and the antisense strand have a length ranging from 16 to 35 nucleotides, respectively. For example, in some embodiments, the sense strand and the antisense strand, independently comprise a length of 16, 17, 18, 19, 20, 21, 22, 23, 24, 25, 26, 27, 28, 29, 30, 31, 32, 33, 34 or 35 nucleotides.

In certain embodiments, one strand of the saRNA has at least 75% (e.g., at least about 79%, about 80%, about 85%, about 90%, about 95% or about 99%) sequence homology or complementarity to a nucleotide sequence selected from SEQ ID NOs: 1-286. Specifically, the sense strand of the saRNA disclosed herein has at least 75% (e.g., at least about 79%, about 80%, about 85%, about 90%, about 95% or about 99%) sequence homology to any nucleotide sequence selected from SEQ ID NOs: 287-858, and the antisense strand of the saRNA disclosed herein has at least 75% (e.g., at least about 79%, about 80%, about 85%, about 90%, about 95% or about 99%) sequence homology to any nucleotide sequence selected from SEQ ID NOs: 859-1430. More specifically, the sense strand of the saRNA disclosed herein comprises or consists of any nucleotide sequence selected from SEQ ID NOs: 287-858; and the antisense strand of the saRNA disclosed herein comprises or consists of or is any nucleotide sequence selected from SEQ ID NOs: 859-1430.

In certain embodiments, one strand of the saRNA has five or less, i.e., 5, 4, 3, 2, 1, or 0 nucleotide differences or mismatches relative to the nucleotide sequence selected from SEQ ID NOs: 1-286. Specifically, the sense strand of the saRNA disclosed herein has five or less, i.e., 5, 4, 3, 2, 1, or 0 nucleotide differences relative to the nucleotide sequence selected from SEQ ID NOs: 287-858, and the antisense strand of the saRNA disclosed herein has five or less, i.e., 5, 4, 3, 2, 1, or 0 nucleotide differences relative to the nucleotide sequence selected from SEQ ID NOs: 859-1430. In some embodiments, the differences or mismatches are located in the middle or 3′ terminus of the sense or antisense strand of the saRNA.

In certain embodiments, the antisense strand disclosed herein is capable of interact with a target nucleic acid sequence of a promoter of a gene in a sequence specific manner, meaning that the antisense strand is capable of undergoing hybridization to a target nucleic acid through hydrogen bonding. In certain embodiments, an antisense strand has a nucleotide sequence that, when written in the 5′ to 3′ direction, comprises the reverse complement of the target portion of a target nucleic acid to which it is targeted. In certain such embodiments, an antisense strand has a nucleotide sequence that, when written in the 5′ to 3′ direction, comprises the reverse complement of the target portion in SEQ ID NO: 1437, specifically, the target portion is a nucleic acid sequence selected from SEQ ID NOs: 1-286.

In the saRNAs disclosed herein, all nucleotides may be natural or non-chemically modified nucleotides, or at least one nucleotide is a chemically modified nucleotide. Non-limiting examples of the chemical modification include one or more of a combination of the following:
(1) modification of a phosphodiester bond of nucleotides in the nucleotide sequence of the saRNA;
(2) modification of 2′-OH of the ribose in the nucleotide sequence of the saRNA;
(3) modification of a base in the nucleotide of the saRNA; and
(4) at least one nucleotide in the nucleotide sequence of a small activating nucleic acid molecule being a locked nucleic acid.

The chemical modification described herein is well-known to those skilled in the art, and the modification of the phosphodiester bond refers to the modification of oxygen in the phosphodiester bond, including phosphorothioate modification and boranophosphate modification. The modifications disclosed herein stabilize a saRNA structure, maintaining high specificity and high affinity for base pairing.

In some embodiments, the saRNA of the present application includes at least one chemically modified nucleotide which is modified at 2′-OH in pentose of a nucleotide, i.e., the introduction of certain substituents at the hydroxyl position of the ribose, such as 2′-fluoro modification, 2′-oxymethyl modification, 2′-oxyethylidene methoxy modification, 2,4′-dinitrophenol modification, locked nucleic acid (LNA), 2′-amino modification or 2′-deoxy modification, e.g., a 2′-deoxy-2′-fluoro modified nucleotide, a 2′-deoxy-modified nucleotide.

In some embodiments, the saRNA of the present application includes at least one chemically modified nucleotide which is modified at the base of the nucleotide, e.g., 5′-bromouracil modification, 5′-iodouracil modification, N-methyluracil modification, or 2,6-diaminopurine modification.

In some embodiments, the chemical modification of the saRNA is an addition of a (E)-vinylphosphonate moiety at the 5′ end of the sense or antisense sequence. In some embodiments, the chemical modification of the at least one chemically modified nucleotide is an addition of a 5′-methyl cytosine moiety at the 5′ end of the sense or antisense sequence.

In some embodiments, the saRNA of the present application includes at least one nucleotide in the nucleotide sequence of the small activating nucleic acid molecule being a chemically modified nucleic acid, e.g., a locked nucleotide, an abasic nucleotide, a 2′-amino-modified nucleotide, a 2′-alkyl-modified nucleotide, a morpholino nucleotide, a phosphoramidate, and a non-natural base comprising nucleotide. In some embodiments, the saRNA disclosed herein includes an “endo-light” modification with 2′-O-methyl modified nucleotides and nucleotides comprising a 5′-phosphorothioate group.

In some embodiments, the saRNA of the present application is chemically modified to enhance stability or other beneficial characteristics. The nucleic acids featured in the present application may be synthesized and/or modified by conventional methods, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of saRNA molecules that can be used in this present application include, but are not limited to RNAs containing modified backbones or no natural internucleoside linkages. In some embodiments, RNAs having modified backbones include, among others, those that do not have a phosphorus atom in the backbone. In some embodiments, modified RNAs that do not have a phosphorus atom in their internucleoside backbone can also be considered to be oligonucleosides. In some embodiments, the modified oligonucleotide will have a phosphorus atom in its internucleoside backbone.

Modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included.

Non-limiting examples of preparation of the phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39464, which are hereby incorporated by reference in their entireties.

In addition, to facilitate entry of the saRNA into a cell, chemical conjugation moieties may be introduced at the ends of the sense or antisense strands of the saRNA on the basis of the above modifications to facilitate action through a cell membrane composed of lipid bilayers and gene promoter regions within the nuclear membrane and nucleus.

In certain embodiments, saRNAs disclosed in the present application are covalently attached to one or more conjugate moieties. In certain embodiments, conjugation moieties modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugation moieties impart a new property on the attached oligonucleotide, e.g., fluorophores or reporter groups that enable detection of the oligonucleotide. Certain conjugation moieties and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO 1, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In some embodiments, the saRNA of the present application relates to the sense strand or the antisense strand of the saRNA that is conjugated to one or more conjugation moieties selected from: intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In some embodiments, a conjugation moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

In some embodiments, the saRNA of the present application is conjugated to one or more conjugation moieties selected from: a lipid, a fatty acid, a fluorophore, a ligand, a saccharide, a peptide, and an antibody.

In some embodiments, the saRNA of the present application relates to the sense strand or the antisense strand of the saRNA that is conjugated to one or more conjugation moieties selected from a cell-penetrating peptide, polyethylene glycol, an alkaloid, a tryptamine, a benzimidazole, a quinolone, an amino acid, a cholesterol, glucose and N-acetylgalactosamine. In certain embodiments, the saRNA is conjugated to two conjugation moieties. In certain embodiments, the two conjugation moieties are a lipid and an N-acetylgalactosamine. In certain embodiments, one or more conjugation moieties are derived from S9, tC2, tC2x6, C5x5, or combinations thereof, as shown in the present application:

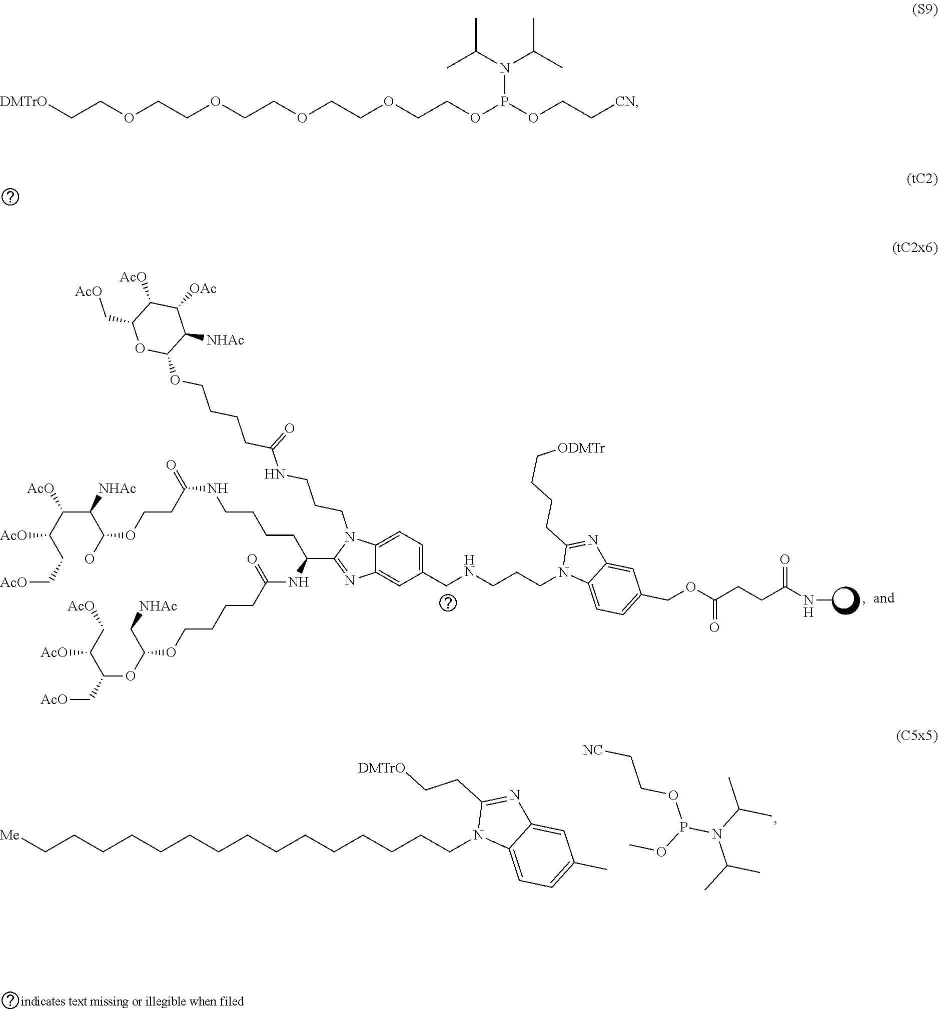

wherein [symbol] represents a support material. In certain embodiments, the conjugation moieties conjugated to the saRNA are S9, tC2x6 and C5x5 as shown in the present application. In certain embodiments, tC2, tC2x6 or S9 conjugates to the 3′ end of sense strand; C5x5 conjugates to the 5′ end of sense strand.

In certain embodiments, said conjugation moiety is a lipid selected from fatty acid comprising a carbon chain length of from 4 to 30 carbon atoms. In certain embodiments, said conjugation moiety is fatty acid comprising a carbon chain length of 16, 18 or 22 carbon atoms. In certain embodiments, the conjugation moiety is selected from lipophilic moieties as described in WO2024002046A1. In certain embodiments, the saRNA may comprise one, two, three, four, five, six or even more oligonucleotides separately conjugated to one, two, three, four, five, six or even more of the conjugation moieties via one, two, three, four, five, six or even more linking moieties.

According to an embodiment, the linking moieties, when present, can be selected from the group consisting of —O—, —S—, —C(O)—, —NH—, —N((C₁-C₁₂)alkyl)-, —N((C₁-C₁₂)alkyl)-C(O)—O—, —O—C(O)—, —C(O)—O—, —O—C(O)—O—, —C(O)—NH—, —OP(O)₂O—, —P(O)(O⁻)O—, —OP(O)O—, —OP(O)(S)O—, —O—S(O)₂—O—, —S(O)₂—O—, —S(O)—O—, —(C₁-C₂₂)alkylene-, —(C₁-C₂₂)alkylene-NH—, —NH—(C₁-C₂₂)alkylene-, —(C₁-C₂₂)alkylene-NH—C(O)—, —(C₁-C₂₂)alkylene-C(O)—, —(C₁-C₂₂)alkylene-C(O)—O—, —C(O)—(C₁-C₂₂)alkylene-, —NH—C(O)—(C₁-C₂₂)alkylene-, —C(O)—NH—(C₁-C₂₂)alkylene-, —C(O)—(C₁-C₂₂)alkylene-NH—, —NH—(C₁-C₂₂)alkylene-C(O)—, —C(O)—(C₁-C₂₂)alkylene-C(O)—, —NH—(C₁-C₂₂)alkylene-NH—, —C(O)—(C₁-C₂₂)alkylene-C(O)O—, —O—C(O)—(C₁-C₂₂)alkylene-C(O)—O—, —C(O)—O—(C₁-C₂₂)alkylene-O—C(O)—, —C(O)—(C₁-C₂₂)alkylene-NH—C(O)—, —NH—C(O)—(C₁-C₂₂)alkylene-C(O)—, —NH—C(O)—(C₁-C₂₂)alkylene-C(O)—NH—, —C(O)—NH—(C₁-C₂₂)alkylene-NH—C(O)—, —(C₁-C₂₂)alkylene-OP(O)₂O—, —(C₁-C₂₂)alkylene-OP(O)(O⁻)O—, —(C₁-C₂₂)alkylene-OP(O)(O⁻)O—(C₁-C₂₂)alkylene-, —(C₁-C₂₂)alkylene-OP(O)O—, —(C₁-C₂₂)alkylene-OP(O)(S)O—, —(C₁-C₂₂)alkylene-O—S(O)₂—O—, —(C₁-C₂₂)alkylene-S(O)₂—O—, —(C₁-C₂₂)alkylene-S(O)—O—, —O—P(O)₂—O—(C₁-C₂₂)alkylene-OP(O)₂O—, —O—P(O)—O—(C₁-C₂₂)alkylene-OP(O)O—, —OP(O)(S)O—(C₁-C₂₂)alkylene-OP(O)(S)O—, —O—S(O)₂—O—(C₁-C₂₂)alkylene-O—S(O)₂—O—, —S(O)₂—O—(C₁-C₂₂)alkylene-S(O)₂—O— and —O—S(O)—(C₁-C₂₂)alkylene-S(O)—O—; wherein the —(C₁-C₂₂)alkylene-contained in the linking moiety can be an alkylene group comprising from 1 to 22 carbon atoms, such as from 2 to 20 carbon atoms, or from 3 to 18 carbon atoms, or from 4 to 16 carbon atoms, or from 5 to 12 carbon atoms, or from 6 to 10 carbon atoms. In one embodiment, the conjugation moiety is directly linked with the oligonucleotide when the linking moiety is a direct bond.

In some embodiments, the saRNA conjugated to one or more conjugation moieties disclosed in the embodiments is directly contacted, transferred, delivered or administrated to a cell or a patient.

In some embodiments, the sense strand and the antisense strand of the saRNA independently have at least about 10%, at least about 20%, at least about 30%, at least about 40%, at least about 50%, at least about 60%, at least about 70%, at least about 80%, at least about 90%, at least about 95%, or about 100% nucleotides which are chemically modified nucleotides.

In some embodiments, at least about 50%, at least about 60%, at least about 70%, at least about 80%, at least about 90%, at least about 95%, or about 100% nucleotides of the saRNA are chemically modified nucleotides.

These modifications can increase the bioavailability of the saRNA, improve affinity to a target sequence, and enhance resistance to nuclease hydrolysis in a cell.

In a non-limiting example, a saRNA is designed based at least in part on the following criteria: (1) GC content between 40% and 70%; (2) no more than 5 consecutive identical nucleotides; (3) no more than 3 dinucleotide or trinucleotide repeats. In some embodiments, a saRNA is design/selected based, at least in part, on criteria that enables production of functional saRNA. For example, in some cases, a sequence located upstream of a TSS may include a sequence that does not favor synthesis of a saRNA despite being located in a hotspot region.

In some embodiments, the saRNA of the present application which, upon contact with a cell, are effective in activating or up-regulating the expression of one or more genes in the cell, preferably by at least 10% (e.g., at least 20%, at least 30%, at least 40%, at least 50%, at least 60%, at least 70%, at least 80%, at least 90%, at least 100%, at least 150%, at least 200%, at least 300%, at least 500%, at least 800%, at least 1000%, at least 2000%, or at least 5000%).

### Target Sequence

In certain embodiments, the present application relates to a target site of the saRNA of the present application, specifically, the target site is a nucleotide sequence or an oligonucleotide having a length ranging from 16 to 35 nucleotides in the nucleotide sequence of SEQ ID NO: 1437. In certain embodiments, the target site or the oligonucleotide is a nucleic acid sequence selected from SEQ ID NOs: 1-286. The target site is capable of interacting with an antisense strand of the saRNA disclosed in the present application, and thus the saRNAs being capable of activating the expression of FVII gene (e.g., mRNA expression, protein expression, FVII expression). In some embodiments, the target site is selected based at least in part on a promoter sequence upstream of the TSS. In some embodiments, the target site is selected based at least in part on a sequence from −5000 bp, −4000 bp, −3000 bp, −2000 bp, −1000 bp or −500 bp upstream of the TSS. In some embodiments, the target site is selected at least in part by moving toward the TSS by 1 bp each time, and resulting in a target sequence, followed by repeating this step and increasing towards the TSS by an additional base pair (e.g., n+1). In some embodiments, the target site has a length of about 8 to about 35 nucleotides. In some embodiments, the target site has a length of about 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides. In some embodiments, the target site or the oligonucleotide is an isolated molecule. In some embodiments, the isolated target site or the isolated oligonucleotide is used for the design, screening and/or preparation of the corresponding saRNA.

In certain embodiments, the present application relates to an oligonucleotide complex comprising the saRNA disclosed herein and the target site disclosed in the present application. In certain embodiments, the oligonucleotide complex activates the expression of FVII gene by at least 10% (e.g., activates expression of the FVII gene as compared to baseline FVII gene expression levels).

### Hotspot

In certain embodiments, the present application relates to a nucleic acid sequence, or namely “hotspot region”, located upstream of the transcription start site of FVII gene. In certain embodiments, the nucleic acid sequence disclosed herein is an oligonucleotide sequence having at least 25 nt, at least 27 nt, at least 30 nt, at least 35 nt, at least 40 nt, at least 45 nt, at least 48 nt or at least 49 consecutive nucleotides in length and has at least 75%, or at least 80%, or at least 85%, or at least 90% sequence homology to an equal length region within the nucleotide sequence of SEQ ID NO: 1437. In some embodiments, the hotspot region is an isolated region.

A “hotspot region” and “hotspot” can be used interchangeably and herein is defined by a nucleic acid region (such as in the promotor upstream the TSS of the target gene) on the target gene of the saRNAs, where full length targets of functional saRNAs are enriched and which span the very 5′ end of the first saRNA's target and the 3′ end of the last saRNA's target within each hotspot. In some embodiments, at least 20% (e.g., 22%, 30%, 40%, 45%, 50%, 55%, 60%, 65%, 70%, 75%, 80%, 85%, 90%, 95% or 100%) of the saRNAs designed to target a sequence within the hotspot is functional, i.e., can induce an at least 1.1-fold change in the mRNA expression of the target gene. In a non-limiting example, at least 20% (such as at least 25%, at least 30%, at least 35%, at least 40%, at least 47%) of the saRNAs designed to target the hotspots is functional, i.e., can induce an at least 1.1-fold change in the mRNA expression of the target gene, provided that the saRNAs are designed based at least in part on the following criteria: (1) GC content between 40% and 70%; (2) no more than 5 consecutive identical nucleotides; (3) no more than 3 dinucleotide or trinucleotide repeats. In some embodiments, the designed functional saRNA can be blunt-ended or with an overhang, and/or without chemical modification(s) or with chemical modification(s).

In some embodiments, the same or similar criteria are used to select a nucleic acid sequence and/or a target sequence. In a non-limiting example, an isolated nucleic acid sequence upstream of the FVII gene's TSS is selected based at least in part on the following criteria: (1) GC content between 40% and 70%; (2) no more than 5 consecutive identical nucleotides; (3) no more than 3 dinucleotides or trinucleotide repeats.

In some embodiments, the nucleic acid region has about 25 to about 250 (e.g., about 33 to about 200, about 36 to about 150, about 39 to about 100, about 42 to about 75, about 45 to about 70, or about 48 to about 55) nucleotides in length. In some embodiments, a hotspot region is a polynucleotide sequence selected from the group consisting of SEQ ID NO: 1438-1441, including any sub-regions in the aforementioned regions as long as it is enriched of the target sequences of the functional saRNAs. In some embodiments, a hotspot region is a nucleic acid sequence selected from the group consisting of region −557 to −379, region −346 to −298, region −271 to −91 and region −96 to −1 upstream of the transcription start site of the FVII gene, including any sub-regions in the aforementioned regions as long as it is enriched of the target sequences of the functional saRNAs. The present application also provides a method of designing saRNA, said method provides saRNA targeting said nucleic acid sequence of the present application.

In some embodiments, a target sequence is designed/selected based, at least in part, on criteria that enables production of functional saRNA. For example, in some cases, a sequence located upstream of a TSS may include a sequence that does not favor synthesis of a target sequence despite being located in a hotspot region.

RNAa activity of each designed saRNA is depended on a complex myriad of factors, such as chromatin environments, sequence features of the target per se and nearby regions, transcriptional factor binding etc. The core underlying determinant may be accessibility of the DNA target. In the regions with higher accessibility, dsRNAs may show a higher activity of RNAa. While dsRNAs designed targeting other regions of the promotor may exhibit non-functional or even transcriptional silencing effect. This may explain the existing of hotspot regions where the targets of the functional saRNAs are clustered together. For example, a target sequence designed based at least in part on the following criteria: (1) GC content between 40% and 70%; (2) no more than 5 consecutive identical nucleotides; (3) no more than 3 dinucleotide or trinucleotide repeats may not activate/up-regulate the expression of FVII gene by at least 10% as compared to baseline expression of the FVII gene because the target sequence that the saRNA binds to is not within a hotspot region (e.g., any of hotspot regions described herein).

In certain embodiments, the present application relates to a nucleic acid complex comprising the saRNA disclosed in the present application and the nucleic acid sequence disclosed herein. In certain embodiments, the complex activates the expression of FVII gene (such as by at least 10%) as compared to baseline expression of the FVII gene.

In some aspects, methods of using the nucleic acid upstream of the transcription target site of FVII gene are also provided.

DNA Encoding saRNA

In certain embodiments, the present application relates to a nucleic acid or polynucleotide encoding the saRNA which can activate or up-regulate the expression of FVII gene in a cell, preferably by at least 10% (e.g., as compared to baseline expression of the FVII gene). In certain embodiments, the nucleic acid is a DNA encoding a saRNA. In certain embodiments, the nucleic acid is a recombinant vector, specifically, a recombinant AAV vector. The vectors disclosed herein comprise a fragment of DNA that encodes a saRNA of the present application.

Cell Comprising saRNA

After contacting a cell, the saRNA disclosed herein can effectively activate or up-regulate the expression of FVII gene in a cell, preferably up-regulate the expression by at least 10% (e.g., as compared to baseline expression of the FVII gene).

In certain embodiments, the present application relates to a cell comprising the saRNA disclosed herein. In some embodiments, the cell is a mammalian cell. In some embodiments, the cell is a human cell, such as a human embryo liver cell, a human hepatoma cell (e.g., a Huh-7 cell), a human hepatoma cell (e.g., a PLC/PRF/5 cell). The cell disclosed herein may be in vitro, or ex vivo, such as a cell line or a cell strain, or may exist in a mammalian body, such as a human body. The human body disclosed herein is a patient suffering from FVII-related disease or symptom, such as those caused by a FVII gene mutation, low FVII level, insufficient levels of functional FVII protein in blood, and/or hemophilia with inhibitor. In some embodiments, the cell is from a patient suffering from hemophilia.

Composition Comprising saRNA

In certain embodiments, the present application relates to a composition or pharmaceutical composition comprising the saRNA or the nucleic acid of the present application. In some embodiments, the composition comprises at least one pharmaceutically acceptable carrier. In some embodiments, the composition comprising at least one pharmaceutically acceptable carrier selected from an aqueous carrier, liposome or LNP, polymer, micelle, colloid, metal nanoparticle, non-metallic nanoparticle, bioconjugate (e.g., GalNAc), polypeptide and antibody. In one embodiment, the aqueous carrier may be, for example, RNase-free water, or RNase-free buffer. In some embodiments, the composition may contain 0.001-1600 nM (e.g., 0.001-1000 nM, 0.001-500 nM, 0.001-400 nM, 10-100 nM, 10-50 nM, 20-50 nM, 20-100 nM, 50-150 nM, 50-400 nM, 50-1000 nM or 400-1600 nM) of the saRNA or polynucleotide as described herein. In some embodiments, the composition includes 25 nM of the saRNA or polynucleotide as described herein. In some embodiments, the composition may contain 0.001-150 nM (e.g., 0.01-100 nM, 0.1-50 nM, 1-150 nM, 1-20 nM, 0.001-1 nM, 1-10 nM, 10-100 nM, 10-50 nM, 20-50 nM, 20-100 nM) of the saRNA or polynucleotide as described herein. In some embodiments, the composition includes 25 nM of the saRNA or polynucleotide as described herein.

Methods of Using saRNA

Another aspect of the present application relates to a method of using saRNA for activating/up-regulating the FVII gene expression in a cell. The saRNA comprises an oligonucleotide sequence having a length of 16 to 35 consecutive nucleotides. In some embodiments, the oligonucleotide sequence has at least 75%, or at least 80%, or at least 85%, or at least 90% homology or complementary to an equal length region of SEQ ID NO: 1437, specifically, the saRNA activates/up-regulates the expression of the FVII gene, such as by at least 10% (e.g., at least 20%, at least 30%, at least 40%, at least 50%, at least 60%, at least 70%, at least 80%, at least 90%, at least 100%, at least 150%, at least 200%, at least 300%, at least 500%, at least 800%, at least 1000%, at least 2000%, or at least 5000% as compared to baseline expression of the FVII gene). In certain embodiments, upon administering the saRNA disclosed in the embodiments, e.g., to a cell or a subject, the expression of the FVII gene is activated/up-regulated by at least 2 fold (e.g., at least 3 fold, at least 4 fold, at least 5 fold, at least 6 fold or at least 7 folds compared to baseline expression of the FVII gene). In certain embodiments, a saRNA activates or up-regulates the expression of the FVII gene by about 6.5-fold. In certain embodiments, the expression of FVII gene is activated/up-regulated by administering the saRNA disclosed in the embodiments to a cell at a concentration of at least 0.01 nM, e.g., 0.02 nM, 0.05 nM, 0.08 nM, 0.1 nM, 0.2 nM, 0.3 nM, 0.4 nM, 0.5 nM, 0.6 nM, 0.8 nM, 1 nM, 5 nM, 10 nM, 25 nM, 50 nM, 75 nM, 100 nM, 150 nM, 200 nM, 400 nM, 800 nM, 1000 nM, or 1600 nM. In certain embodiments, the induction of FVII protein (FVII) is activated/up-regulated by administering the saRNA disclosed in the embodiments to a cell at a concentration of at least 0.01 nM, e.g., 0.02 nM, 0.05 nM, 0.08 nM, 0.1 nM, 0.2 nM, 0.3 nM, 0.4 nM, 0.5 nM, 0.6 nM, 0.8 nM, 1 nM, 2 nM, 3 nM, 4 nM, 5 nM, 10 nM, 25 nM, 50 nM, 75 nM, 100 nM, or 150 nM.

Another aspect of the present application relates to a method for preventing or treating FVII-related disorder or condition, such as those induced by insufficient expression of factor VII (FVII), a FVII gene mutation, low functional FVII levels in blood in an individual and/or those with normal FVII level but can be treated by increasing FVII level, such as hemophilia with inhibitor, comprising: administering an effective amount of the saRNA, the nucleic acid or polynucleotide encoding the saRNA, or the composition comprising the saRNA disclosed herein to the individual. In certain embodiments, the effective amount of the saRNA disclosed herein can be a concentration ranging from 0.01 nM to 1600 nM, e.g., 0.01 nM, 0.02 nM, 0.05 nM, 0.08 nM, 0.1 nM, 0.2 nM, 0.3 nM, 0.4 nM, 0.5 nM, 0.6 nM, 0.8 nM, 1 nM, 5 nM, 10 nM, 25 nM, 50 nM, 75 nM, 100 nM, 150 nM, 200 nM, 400 nM, 800 nM, 1000 nM, or 1600 nM. In certain embodiments, the effective amount of the saRNA disclosed herein can be a concentration ranging from 0.01 nM to 150 nM, e.g., 0.01 nM, 0.02 nM, 0.05 nM, 0.08 nM, 0.1 nM, 0.2 nM, 0.3 nM, 0.4 nM, 0.5 nM, 0.6 nM, 0.8 nM, 1 nM, 5 nM, 10 nM, 25 nM, 50 nM, 75 nM, 100 nM, or 150 nM. In some embodiments, the disorder or condition is hemophilia, such as hemophilia with inhibitor. In some embodiments, the individual is a mammal. In some embodiments, the individual is a human.

In any of the embodiments provided herein, such saRNA, nucleic acids encoding the saRNA of the present application, or compositions comprising such saRNA of the present application may be introduced directly into a cell, or may be produced intracellularly upon introduction of a nucleotide sequence encoding the saRNA into a cell, for example a mammalian cell including, but not limited to, PLC/PRF/5 and Huh-7, or a human cell. Such cells may be ex vivo, such as cell lines, and the like, or may be present in mammalian bodies, such as humans. In some embodiments, the human is a patient or individual suffering from a FVII-deficiency-related condition or hemophilia with inhibitor. In certain embodiments, a nucleic acid or a polynucleotide encoding a saRNA or a composition comprising the aforementioned saRNA as described herein, in respective amounts sufficient to treat hemophilia.

Another aspect of the present application relates administering an effective mount of the saRNA or the composition to an individual using administration pathway as described herein. In some embodiments, the administration pathway is selected from one or more of: parenteral infusions, oral administration, intranasal administration, inhaled administration, vaginal administration, and rectal administration. In some embodiments, the administration pathway is selected from one or more of: intrathecal, intramuscular, intravenous, intra-arterial, intraperitoneal, intravesical, intracerebroventricular, intravitreal and subcutaneous administrations.

### Dose Regiments and Route of Administration

Aspects of the present application relate to a pharmaceutical composition comprising the saRNA of the present application. In some embodiments, the pharmaceutical composition comprising the saRNA of the present application and a pharmaceutically acceptable carrier, a therapeutically inert carrier, diluent or pharmaceutically acceptable excipient. The pharmaceutical composition disclosed herein is to be developed into a medicament preventing or treating the FVII-deficiency-related condition or hemophilia (such as hemophilia with inhibitor).

Aspects of the present application also relate to methods of using the saRNAs of the present application to prepare such compositions.

Another aspect of the present application relates to use of the saRNA of the present application in manufacturing the pharmaceutical composition disclosed herein.

Another aspect of the present application relates to use of the saRNA or a polynucleotide, according to any one of the embodiments described herein, or a composition according to any one of the embodiments described herein, in the manufacture of a medicament for the prevention or treatment of a FVII gene or FVII protein-related symptom, such as that induced by the insufficient expression of FVII protein, a FVII gene mutation, low functional FVII levels in blood in an individual and/or hemophilia (such as hemophilia with inhibitor). The use according to certain embodiments, the condition can include a FVII gene-mutation-related disorder or condition, or hemophilia with inhibitor. The use according to certain embodiments, the symptom is induced by insufficient expression of FVII protein or hemophilia with inhibitor. Also related is the use according to certain embodiments wherein the individual is a mammal, for example a human.

The dosage at which the saRNAs or compositions of the present application can be administered can vary within wide limits and will be fitted to the individual requirements in each case. In certain embodiments, a first dose of a pharmaceutical composition according to the present application is administered when the subject is less than one week old, less than one month old, less than 3 months old, less than 6 months old, less than one-year-old, less than 2 years old, less than 15 years old, or older than 15 years old.

The single dose of the saRNA can be a single dose ranging from 0.01 mg/kg to 1000 mg/kg for example, about 0.01, 0.02, 0.05, 0.1, 0.2, 0.5, 1, 2, 2.5, 5, 7.5, 10, 12.5, 15, 17.5, 20, 25, 30, 40, 50, 75, 100, 120, 150, 200, 250, 300, 400, 500, 750, or 1000 mg/kg. The doses described herein may contain two or more of any of the saRNA sequences described herein.

In some embodiments, the proposed dose frequency is approximate. For example, in certain embodiments if the proposed dose frequency is a dose at day 1 and a second dose at day 29, a patient may receive a second dose 25, 26, 27, 28, 29, 30, 31, 32, 33, or 34 days after receipt of the first dose. In certain embodiments, if the proposed dose frequency is a dose at day 1 and a second dose at day 15, a patient may receive a second dose 10, 11, 12, 13, 14, 15, 16, 17, 18, 19, or 20 days after receipt of the first dose. In certain embodiments, if the proposed dose frequency is a dose at day 1 and a second dose at day 85, a patient may receive a second dose 80, 81, 82, 83, 84, 85, 86, 87, 88, 89, or 90 days after receipt of the first dose.

In certain embodiments, the dose and/or the volume of the injection will be adjusted based on the patient's age, the patient's body weight, and/or other factors that may require adjustment of the parameters of the injection.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may vary considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

Examples of other compositions or components associated with the saRNA, compositions, pharmaceutical compositions, and methods described herein include, but are not limited to: diluents, salts, buffers, chelating agents, preservatives, drying agents, antimicrobials, needles, syringes, packaging materials, tubes, bottles, flasks, beakers, and the like, for example, for using, modifying, assembling, storing, packaging, preparing, mixing, diluting, and/or preserving the components for a particular use. In embodiments where liquid forms of any of the components are used, the liquid form may be concentrated or ready to use.

In some embodiments, lipid moieties used in nucleic acid therapies can be applied in the present application for delivery of the saRNA molecules disclosed herein. In such methods, the nucleic acid (e.g., one or more saRNAs described herein) is introduced into preformed liposomes or lipoplexes made of mixtures of cationic lipids and neutral lipids. In certain methods, saRNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes and/or coated with a tissue-specific antibody.

In some embodiments, the saRNA can be delivered or administered via a vector. Any vectors that may be used for gene delivery may be used. In some embodiments, a viral vector may be used. Non-limiting examples of viral vectors that may be used in the present application include, but are not limited to, human immunodeficiency virus; HSV, herpes simplex virus; MMSV, Moloney murine sarcoma virus; MSCV, murine stem cell virus; SFV, Semliki Forest virus; SIN, Sindbis virus; VEE, Venezuelan equine encephalitis virus; VSV, vesicular stomatitis virus; VV, vaccinia virus; AAV, adeno-associated virus; adenovirus; lentivirus; and retrovirus.

In some embodiments, the vector is a recombinant AAV vector (rAAV). AAV vectors are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

Preparations, pharmaceutical compositions, or medicaments of the present application are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

For the preparations, pharmaceutical compositions, or medicaments of the present application, the delivery can be optionally through parenteral infusions including intrathecal, intramuscular, intravenous, intra-arterial, intraperitoneal, intravesical, intracerebroventricular, intravitreal or subcutaneous administration; or through oral administration, intranasal administration, inhaled administration, vaginal administration, or rectal administration.

A typical formulation of the oligonucleotide modulator in the present application is prepared by mixing a saRNA of the present application and a carrier or excipient. Suitable carriers and excipients are well known to those skilled in the art and are described in detail in, e.g., Ansel H. C. et al., Ansel's Pharmaceutical Dosage Forms and Drug Delivery Systems (2004) Lippincott, Williams & Wilkins, Philadelphia; Gennaro A. R. et al., Remington: The Science and Practice of Pharmacy (2000) Lippincott, Williams & Wilkins, Philadelphia; and Rowe R. C, Handbook of Pharmaceutical Excipients (2005) Pharmaceutical Press, Chicago. The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents, diluents and other known additives to provide an elegant presentation of the drug (i.e., a saRNA of the present application or pharmaceutical composition thereof) or aid in the manufacturing of the pharmaceutical product (i.e., medicament).

### Method of Diagnosis

Another aspect of the present application relates to a method for detecting FVII protein or FVII regulated protein in blood. In certain embodiments, the method includes detecting FVII protein or FVII regulated protein in a cell transfected with the saRNA, the polynucleotide, or the composition comprising the saRNA as disclosed herein. In certain embodiments, the method disclosed herein can be applied in detecting a specific sub-group of patients suffering a disorder or condition induced by insufficient expression of factor VII (FVII) protein, a FVII gene mutation, low functional FVII levels in blood and/or hemophilia with inhibitor. As alternative embodiments of the method disclosed herein, the method can be used in efficacy or safety monitoring of the aforementioned patients treated by the saRNA, nucleic acid or polynucleotide encoding the saRNA, composition, or medicament of the present application.

In certain embodiments, a baseline measurement is obtained from a biological sample, as defined herein, obtained from an individual prior to administering the therapy described herein. In certain embodiments, a baseline expression of the FVII gene is obtained from a biological sample prior to administering the saRNA described herein. In certain embodiments, the biological sample is peripheral blood mononuclear cells, blood plasma, serum, skin tissue or part of an organ.

In some embodiments, the saRNA provided herein activates the amount of functional FVII protein in blood as compared to the baseline measurement aforementioned, by at least 10% (e.g., at least 20%, at least 30%, at least 40%, at least 50%, at least 60%, at least 70%, at least 80%, at least 90%, at least 100%, at least 150%, at least 200%, at least 300%, at least 500%, at least 800%, at least 1000%, at least 2000%, or at least 5000%).

In some embodiments, the saRNA shows a greater than additive effect or synergy in the treatment, prevention, delaying progression and/or amelioration of diseases caused by the FVII gene mutation. In some embodiments, the saRNA shows a greater than additive effect or synergy in the protection of cells implicated in the pathophysiology of the disease, particularly for the treatment, prevention, delaying progression and/or amelioration hemophilia.

Another aspect of the present application relates to a method for activating/up-regulating expression of FVII gene in a cell comprising: administering the saRNA, or the polynucleotide, or the composition of the embodiments disclosed herein. In some embodiments, the saRNA, or the polynucleotide, or the composition is introduced into the cell. In some embodiments, the saRNA of the embodiments disclosed herein is produced in the cell after a nucleotide sequence encoding the saRNA is introduced into the cell. In some embodiments, the cell disclosed herein is a mammalian cell, preferably a human cell.

Another aspect of the present application relates to a method for increasing a level of FVII protein in a cell or a level of functional FVII protein in blood of a patient, comprising introducing an effective amount of the saRNA, the nucleic acid or polynucleotide encoding the saRNA, or the composition of the embodiments disclosed herein into the cell or subject.

### Kit

Another aspect of the present application relates to a kit for performing the method for increasing a level of FVII protein in a cell or a level of functional FVII protein in blood, comprising the saRNA disclosed herein. In certain embodiments, the kit further comprises means for administering said saRNA to an individual. In certain embodiments, the kit is in a labeled package and the label on said package indicates that the saRNA or the composition can be used in preventing or treating FVII-related disease or condition or disorder, such as the one induced by insufficient expression of factor VII (FVII), or against hemophilia (such as hemophilia with inhibitor).

A “kit” as used herein, typically defines a package, assembly, or container (such as an insulated container) including one or more of the components or embodiments of the application, and/or other components associated with the application, for example, as previously described. Any of the agents or components of the kit may be provided in liquid form (e.g., in solution), or in solid form (e.g., a dried powder, frozen, etc.).

In additional embodiments, a kit can include instructions or instructions to a website or other source in any form that are provided for using the kit in connection with the components and/or methods described herein. For instance, the instructions may include instructions for the use, modification, mixing, diluting, preserving, assembly, storage, packaging, and/or preparation of the components and/or other components associated with the kit. In some cases, the instructions may also include instructions for the delivery of the components, for example, for shipping or storage at room temperature, sub-zero temperatures, cryogenic temperatures, etc.

Another aspect of the present application relates to a kit for detecting FVII protein or FVII regulated protein in blood. In certain embodiments, the kit is for detecting FVII protein or FVII regulated protein in a cell transfected with any one or more of the saRNA disclosed herein, or the polynucleotide, or the composition disclosed herein. Also provided herein is a kit for increasing level of FVII protein in a cell.

### PARTICULAR EMBODIMENTS

The present application provides the following particular embodiments:

In some aspects, provided herein are saRNAs, in particular:
1. A small activating RNA (saRNA) comprising a sense strand and an antisense strand, wherein:
   each of the sense strand or the antisense strand of the saRNA comprises a consecutive oligonucleotide sequence of 16 to 35 consecutive nucleotides, wherein the consecutive oligonucleotide sequence independently has at least 75%, at least 80%, at least 85%, at least 90%, at least 95%, at least 98% or 100% homology or complementarity to an equal length consecutive fragment of SEQ ID NO: 1437; and wherein
   the saRNA increases the expression of FVII gene by at least 10% as compared to baseline expression of FVII gene.
2. The saRNA of embodiment 1, wherein the equal length consecutive fragment of SEQ ID NO: 1437 is located in a region upstream of the transcription start site (TSS) of FVII gene, and wherein
   the equal length consecutive fragment of SEQ ID NO: 1437 is located in a region selected from the group consisting of: region −557 to −379; region −346 to −298; region −271 to −91; and, region −96 to −1; and/or
   the equal length consecutive fragment of SEQ ID NO: 1437 is located in a region of SEQ ID NO: 1438, SEQ ID NO: 1439, SEQ ID NO: 1440, or SEQ ID NO: 1441.
3. The saRNA of embodiment 1 or 2, wherein the equal length consecutive fragment of SEQ ID NO: 1437 is selected from SEQ ID NO: 1-286.
4. The saRNA of any one of embodiments 1-3, wherein the equal length consecutive fragment of SEQ ID NO: 1437 is selected from SEQ ID NOs: 37, 55, 77, 86, 116, 117, 120, 124, 145, 146, 151, 177, 188, 205, 207, 218, 228, 241, 242, 10, 11, 29, 88, 121, 156, 259, 261, 274, 9, 27, 69, 81, 82, 107, 240 and 252.
5. The saRNA of any one of embodiments 1-4, wherein the consecutive oligonucleotide sequence has (1) GC content between 40% and 70%; (2) no more than 5 consecutive identical nucleotides; (3) no more than 3 dinucleotide or trinucleotide repeats; and/or the consecutive oligonucleotide sequence is complementary to the sense strand of the saRNA or to the antisense strand of the saRNA.
6. The saRNA of any one of embodiments 1-5, wherein the sense strand and the antisense strand independently has a length of about 16 to about 35, about 17 to about 30, about 18 to about 25, or about 19 to about 22 nucleotides.
7. The saRNA of any one of embodiments 1-6, wherein the sense strand and the antisense strand have a complementarity of at least 90%; and/or
   wherein the sense strand and the antisense strand are located on two different nucleic acid strands or on a contiguous nucleic acid strand; and/or
   wherein the consecutive oligonucleotide sequence comprises 0, 1, 2, or 3 mismatches to the complementary region of the sense strand or the complementary region of the antisense strand.
8. The saRNA of any one of embodiments 1-7, wherein the sense strand and the antisense strand each comprises a complementary region, and wherein the complementary regions of the sense strand and the antisense strand form a double-stranded nucleic acid structure.
9. The saRNA of embodiment 8, wherein the sense strand or the antisense strand comprises a 3′ overhang which is independently 1-6, 1-5, or 2-3 nucleotides in length; or the double-stranded nucleic acid structure is blunt-ended.
10. The saRNA of embodiment 9, wherein at least one of the nucleotides of the overhang is thymine deoxyribonucleotide (dT).
11. The saRNA of any one of embodiments 1-10, wherein the consecutive oligonucleotide sequence of the sense strand has at least 75% sequence homology to a nucleotide sequence selected from SEQ ID NOs: 287-858, and/or
   the consecutive oligonucleotide sequence of the antisense strand has at least 75% sequence homology to a nucleotide sequence selected from SEQ ID NOs: 859-1430.
12. The saRNA of any one of embodiments 1-11, wherein the consecutive oligonucleotide sequence of the sense strand is selected from SEQ ID NOs: 287-858, and the consecutive oligonucleotide sequence of the antisense strand is selected from SEQ ID NOs: 859-1430.
13. The saRNA of any one of embodiments 1-12, wherein when the consecutive oligonucleotide sequence of the sense strand is as set forth in SEQ ID NO: n, the consecutive oligonucleotide sequence of the antisense strand is as set forth in SEQ ID NO: n+572 or n+858, wherein n is an integer selected from 287-572; and/or, wherein when the consecutive oligonucleotide sequence of the sense strand is as set forth in SEQ ID NO: n′, the consecutive oligonucleotide sequence of the antisense strand is as set forth in SEQ ID NO: n′+286, wherein n′ is an integer selected from 573-858.
14. The saRNA of any one of embodiments 1-13, wherein the sense strand comprises a consecutive nucleotide sequence selected from SEQ ID NOs: 323, 341, 363, 372, 402, 403, 406, 410, 431, 432, 437, 463, 474, 491, 493, 504, 514, 527, 528, 296, 297, 315, 374, 407, 442, 545, 547, 560, 581, 582, 599, 627, 641, 653, 654, 658, 679, 749, 812, 813, and 824; and/or
   the antisense strand comprises a consecutive nucleotide sequence selected from SEQ ID NOs: 859, 913, 935, 944, 974, 975, 978, 982, 1003, 1004, 1009, 1035, 1046, 1063, 1065, 1076, 1086, 1099, 1100, 1154, 1155, 1173, 1232, 1265, 1268, 1300, 1349, 1351, 1362, 1372, 1385, 1403, 1405, 1418, 867, 868, 885, 927, 939, 940, 965, 1098, and 1110.
15. The saRNA of any one of embodiments 1-14, wherein the sense strand comprises a nucleotide sequence of SEQ ID NO: m, and the antisense strand comprises a nucleotide sequence of SEQ ID NO: m+572, and wherein m is selected from 323, 341, 363, 372, 402, 403, 406, 410, 431, 432, 437, 463, 474, 491, 493, 504, 514, 527, 528; and/or
   wherein the sense strand comprises a nucleotide sequence of SEQ ID NO: m′, and the antisense strand comprises a nucleotide sequence of SEQ ID NO: m+858, and wherein m is selected from 296, 297, 315, 374, 407, 410, 442, 491, 493, 504, 514, 527, 545, 547, 560; and/or, wherein the sense strand comprises a nucleotide sequence of SEQ ID NO: m″, and the antisense strand comprises a nucleotide sequence of SEQ ID NO: m″+286, and wherein in is selected from 581, 582, 599, 627, 641, 653, 654, 658, 679, 749, 812, 813, and 824.
16. The saRNA of any one of embodiments 1-15, wherein the sense strand comprises a consecutive nucleotide sequence selected from SEQ ID NOs: 1442, 1444, 1446, 1448, 1450, 1452, 1454, 1456, 1458, 1460, 1462, 1464, 1466, 1468, 1470, 1472, 1474 and 1476; and/or
   the antisense strand comprises a consecutive nucleotide sequence selected from SEQ ID NOs: 1443, 1445, 1447, 1449, 1451, 1453, 1455, 1457, 1459, 1461, 1463, 1465, 1467, 1469, 1471, 1473 and 1475; and/or
   the saRNA comprises the sense strand and the antisense strand to form a duplex selected from any of the duplexes listed in Table 11.
17. The saRNA of any one of embodiments 1-16, wherein at least one nucleotide of the saRNA is a chemically modified nucleotide.
18. The saRNA of embodiment 17, wherein the chemically modified nucleotide is a nucleotide with at least one the following modifications:
a) modification of a phosphodiester bond connecting nucleotides in the nucleotide sequence of the saRNA;
b) modification of 2′-OH of a ribose in the nucleotide sequence of the saRNA; and
c) modification of a base in the nucleotide sequence of the saRNA.
19. The saRNA of embodiment 18, wherein the modification of a phosphodiester bond connecting nucleotides is selected from a phosphorothioate modification and boranophosphate modification; and/or
   the modified of 2′-OH is selected from the group consisting of 2′-fluoro modification, 2′-oxymethyl modification, 2′-oxyethylidene methoxy modification, 2,4′-dinitrophenol modification, 2′-amino modification and 2′-deoxy modification; and/or
   the modification of a base is selected from the group consisting of 5′-bromouracil modification, 5′-iodouracil modification, N-methyluracil modification, and 2,6-diaminopurine modification.
20. The saRNA of embodiment 17, wherein at least one nucleotide of the saRNA is a locked nucleic acid, an abasic nucleotide, a 2′-amino-modified nucleotide, a 2′-alkyl-modified nucleotide, a morpholino nucleotide, a phosphoramidate, or a non-natural base comprising nucleotide; and/or wherein the chemical modification of the at least one chemically modified nucleotide is an addition of a (E)-vinylphosphonate moiety at the 5′ end of the sense strand or the antisense strand.

In some aspects, provided herein are oligonucleotide modulators, in particular:
21. An oligonucleotide modulator comprising one or more saRNA according to any one of embodiments 1-20.
22. The oligonucleotide modulator of embodiment 21, further comprising one or more moieties or components conjugated, combined or mixed with said saRNA(s).
23. The oligonucleotide modulator of embodiment 21, wherein the sense strand and/or the antisense strand of the saRNA is conjugated to one or more conjugation moieties selected from the group consisting of a lipid, a fatty acid (such as fatty acid comprising a carbon chain with 4-30, 12-24, 16-22 carbon atoms), a fluorophore, a ligand, a saccharide, a peptide, and an antibody.
24. The oligonucleotide modulator of embodiment 22, wherein the conjugation moiety is independently selected from a lipid, a cell-penetrating peptide, a polyethylene glycol, an alkaloid, a tryptamine, a benzimidazole, a quinolone, an amino acid, a cholesterol, a glucose, a N-acetylgalactosamine, and any combinations thereof.
25. The oligonucleotide modulator of embodiment 22, wherein the conjugation moieties is independently selected from S9, tC2, tC2x6, C5x5, and any combinations thereof,

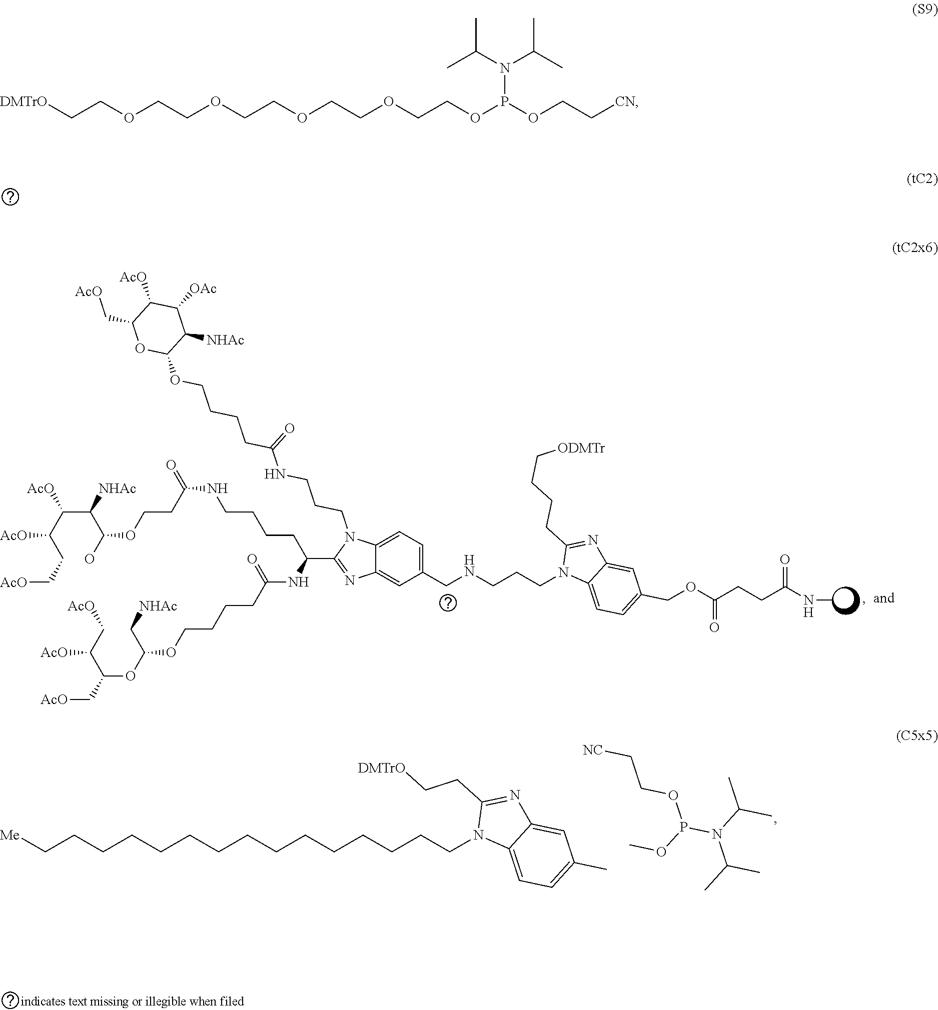

wherein [symbol] represents a support material.
26. The oligonucleotide modulator of embodiment 21, wherein the oligonucleotide modulator further comprises a saRNA conjugated to or combined with one or more of other active moieties for FVII related disease or condition or disorder treatment, wherein the one or more of other active moieties are independently selected from a small molecule chemical moiety, a polypeptide and an antibody.

In some aspects, provided herein are target sites, in particular:
27. An isolated oligonucleotide, wherein (a) the isolated oligonucleotide is a consecutive oligonucleotide sequence of 16-35 consecutive nucleotides having at least 75%, at least 80%, at least 85%, at least 90%, at least 95%, at least 98% or 100% homology to an equal length consecutive fragment of SEQ ID NO: 1437; or
wherein (b) the isolated oligonucleotide is a consecutive oligonucleotide sequence of 16-35 consecutive nucleotides having at least 75%, at least 80%, at least 85%, at least 90%, at least 95%, at least 98% or 100% complementarity to an equal length consecutive fragment of SEQ ID NO: 1437.
28. The isolated oligonucleotide of embodiment 27, wherein the equal length consecutive fragment of SEQ ID NO: 1437 is located in a region upstream of the transcription start site (TSS) of FVII gene, and wherein
   the equal length consecutive fragment of SEQ ID NO: 1437 is located in a region selected from the group consisting of: region −557 to −379; region −346 to −298; region −271 to −91; and, region −96 to −1; and/or
   the equal length consecutive fragment of SEQ ID NO: 1437 is located in a region of SEQ ID NO: 1438, SEQ ID NO: 1439, SEQ ID NO: 1440, or SEQ ID NO: 1441.
29. The isolated oligonucleotide of embodiment 27, wherein the isolated oligonucleotide (a) is a nucleic acid sequence selected from SEQ ID NOs: 1-286, such as selected from SEQ ID NOs: 37, 55, 77, 86, 116, 117, 120, 124, 145, 146, 151, 177, 188, 205, 207, 218, 228, 241, 242, 10, 11, 29, 88, 121, 156, 259, 261, 274, 9, 27, 69, 81, 82, 107, 240 and 252; or
the isolated oligonucleotide (b) is a nucleic acid sequence complementary to a nucleic acid sequence selected from SEQ ID NOs: 1-286, such as complementary to SEQ ID NOs: 37, 55, 77, 86, 116, 117, 120, 124, 145, 146, 151, 177, 188, 205, 207, 218, 228, 241, 242, 10, 11, 29, 88, 121, 156, 259, 261, 274, 9, 27, 69, 81, 82, 107, 240 and 252;
30. An oligonucleotide complex comprising:
   the antisense strand of the saRNA of any of embodiment 1-20 and the sense strand of the isolated oligonucleotide (a) of any of embodiment 27-29; or
   the sense strand of the saRNA of any of embodiment 1-20 and the antisense strand of the isolated oligonucleotide (b) of any of embodiment 27-29.
31. The oligonucleotide complex of embodiment 30, wherein the oligonucleotide complex activates the expression of FVII gene by at least 10% as compared to baseline expression of the FVII gene.

In some aspects, provided herein are hotspots, in particular:
32. An isolated nucleic acid molecule, wherein at least 20% (such as at least 30%, at least 40%, at least 45%, at least 50%) of saRNAs designed to target the sequence of the isolated nucleic acid molecule activate the expression of FVII gene by at least 10%, wherein the designed saRNA has (1) GC content between 40% and 70%; (2) no more than 5 consecutive identical nucleotides; (3) no more than 3 dinucleotide or trinucleotide repeats.
33. An isolated nucleic acid molecule having a sequence corresponding to a region upstream of the transcription start site of the FVII gene, wherein the sequence is located on the region selected from region −557 to −379, region −346 to −298, region −271 to −91 and region −96 to −1 or any sub-regions in any of the above regions, upstream of the transcription start site of the FVII gene.
34. An isolated nucleic acid molecule having a sequence selected from SEQ ID NOs: 1438-1441.
35. The isolated nucleic acid molecule of any one of embodiments 32-34 comprising the isolated oligonucleotide of embodiment 27(a) or 29(a).
36. The isolated nucleic acid molecule of any one of embodiments 32-34, wherein designed saRNAs targeting the nucleic acid molecule are as defined in any one of embodiments 1-20; and/or the desired saRNA is blunt ended or with an overhang, and/or without chemical modification(s) or with chemical modification(s).

In some aspects, provided herein are DNAs, in particular:
37. An isolated polynucleotide encoding the saRNA of any one of embodiments 1-20 or the oligonucleotide modulator of any one of embodiments 21-26.
38. The isolated polynucleotide of embodiment 37, wherein the isolated polynucleotide is a DNA.
39. A vector comprising the isolated polynucleotide of any one of embodiments 37-38.

In some aspects, provided herein are cells, in particular:
40. A host cell comprising the saRNA of any one of embodiments 1-20, the oligonucleotide modulator of any one of embodiments 21-26, the isolated polynucleotide of any one of embodiments 37-38, or the vector of embodiment 39.

In some aspects, provided herein are products, in particular:
41. A product comprising the saRNA of any one of embodiments 1-20, the oligonucleotide modulator of any one of embodiments 21-26, the isolated oligonucleotide of any one of embodiments 27-29, the oligonucleotide complex of any one of embodiments 30-31, the isolated nucleic acid molecule of any one of embodiments 32-36, the isolated polynucleotide of any one of embodiments 37-38, the vector of embodiment 39 or the host cell of embodiment 40.
42. The product of embodiment 41, wherein the product is a product for prevention, treatment or diagnosis of FVII-related disease or disorder or condition (such as a FVII defect associated disease or hemophilia), for saRNA designing and/or screening.

In some aspects, provided herein are pharmaceutical compositions, in particular:
43. A composition comprising the saRNA of any one of embodiments 1-20, the oligonucleotide modulator of any one of embodiments 21-26, or the isolated polynucleotide of embodiment 37-38 and optionally, a pharmaceutically acceptable carrier.
44. The composition of embodiment 43, wherein the composition comprises 0.001-1600 nM, such as 1-150 nM of the saRNA.

In some aspects, provided herein are products for use and/or use in preparation of a medicament for activating/up-regulating FVII gene expression, in particular:
45. A product for activating/up-regulating FVII gene expression in a cell, wherein the product activates the expression of FVII gene by at least 10% as compared to baseline expression of the FVII gene, and wherein the product comprises an active substance selected from one or more of the saRNA of any one of embodiments 1-20 or an oligonucleotide modulator of any one of embodiments 21-26, the isolated polynucleotide of any one of embodiments 37-38, the vector of embodiment 39, or the composition of any one of embodiments 43-44.
46. Use of an active substance in the preparation of a product for activating/up-regulating FVII gene expression in a cell, wherein the product activates the expression of FVII gene by at least 10% as compared to baseline expression of the FVII gene, and wherein the active substance is selected from one or more of the saRNA of any one of embodiments 1-20 or an oligonucleotide modulator of any one of embodiments 21-26, the isolated polynucleotide of any one of embodiments 37-38, the vector of embodiment 39, or the composition of any one of embodiments 43-44.
47. A method for activating/up-regulating FVII gene expression in a cell, wherein the product activates the expression of FVII gene by at least 10% as compared to baseline expression of the FVII gene, and wherein the method comprises administering an effective amount of an active substance to a cell, wherein the active substance is selected from one or more of the saRNA of any one of embodiments 1-20 or an oligonucleotide modulator of any one of embodiments 21-26, the isolated polynucleotide of any one of embodiments 37-38, the vector of embodiment 39, or the composition of any one of embodiments 43-44.
48. The product for activating/up-regulating FVII gene expression in a cell of embodiment 45, the use of embodiment 46 or the method of embodiment 47, wherein the active substance is introduced into the cell; and/or
   wherein the cell is in vitro, ex vivo or in vivo; and/or
   wherein the cell is a mammalian cell.
49. The product, the use or the method of embodiment 48, wherein the active substance is introduced into the cell by:
   1) composing the active substance with a physiologically acceptable or pharmaceutically acceptable carrier, such as one or more selected from the group consisting of an aqueous carrier, a liposome, a high-molecular polymer, a polypeptide and an antibody, and/or
   2) conjugating the active substance to one or more conjugation moieties, such as one or more selected from a lipid, a cell-penetrating peptide, a polyethylene glycol, an alkaloid, a tryptamine, a benzimidazole, a quinolone, an amino acid, a cholesterol, a glucose, and a N-acetylgalactosamine, and any combinations thereof (for example two conjugation moieties wherein one is a lipid and the other is a N-acetylgalactosamine).
50. The product, the use or the method of embodiment 49, wherein said conjugation moiety is one or more selected from S9, tC2, tC2x6, and C5x5, or any combinations thereof (such as C5x5 and tC2x6):

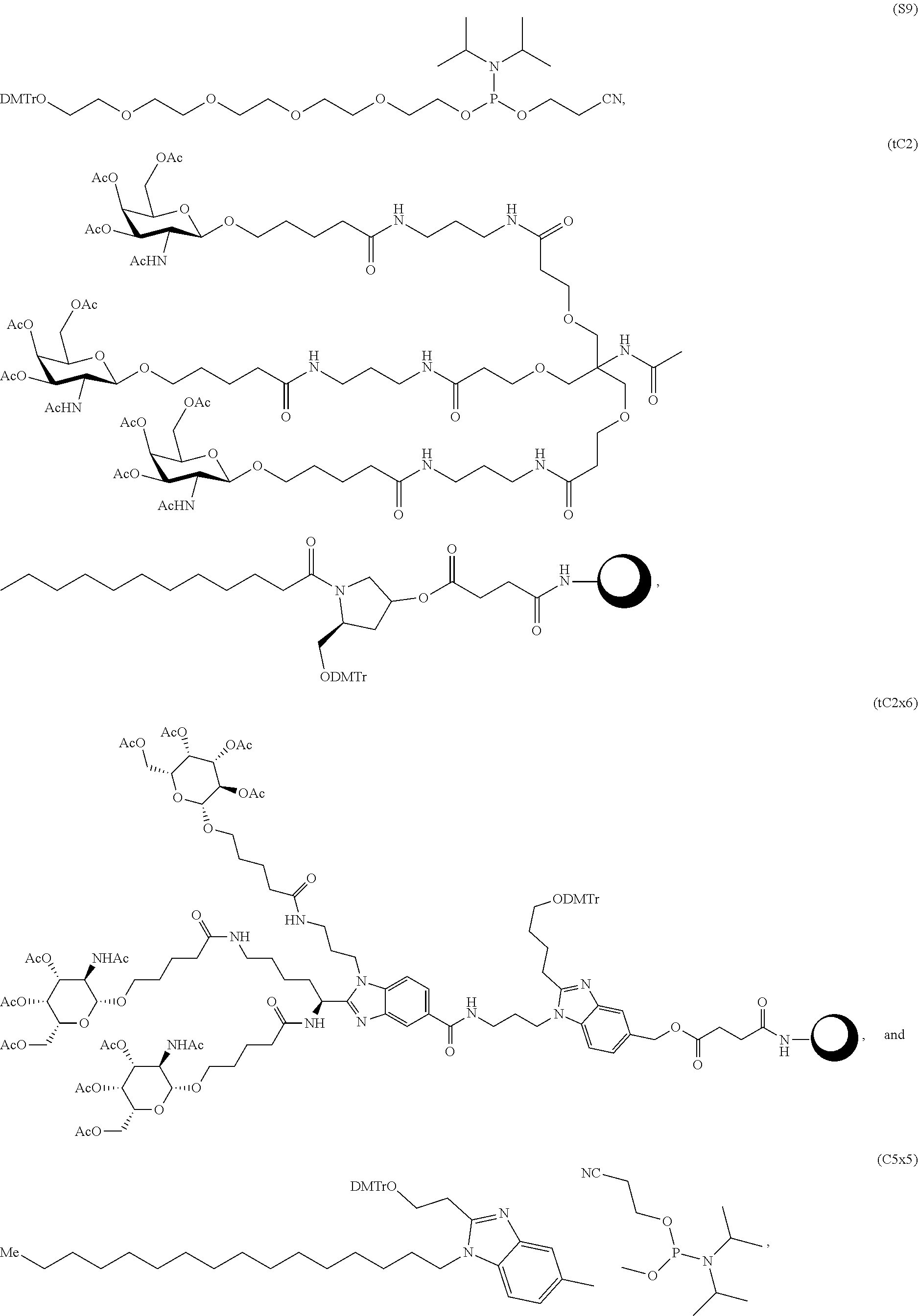

wherein [symbol] represents a support material.
51. The product, the use or the method of embodiment 49, wherein said conjugation moiety is a lipid selected from fatty acid comprising a carbon chain length of from 4 to 30, 12-24, 16-22 carbon atoms; and/or
wherein said conjugation moiety is fatty acid having a carbon chain length of 4-30, 12-24, 16-22, or 16 carbon atoms; and/or
wherein the conjugation moiety is independently derived from a fluorophore, a ligand, a saccharide, a peptide, and an antibody.
52. The product for activating/up-regulating FVII gene expression in a cell of embodiment 45, the use of embodiment 46 or the method of embodiment 47, wherein the cell is from a patient suffering from or in risk of having a disease or condition or disorder induced by insufficient expression of the FVII protein, a FVII gene mutation, low functional FVII levels in blood, and/or other diseases or conditions or disorders preventable or treatable by activating/up-regulating FVII gene expression, such as hemophilia (e.g., hemophilia with inhibitor) or Glanzmann's thrombasthenia (GT), wherein the active substance is administered in a sufficient amount to prevent or treat the disease or condition or disorder.

In some aspects, provided herein are product for use in method for/use in preparation of a medicament for treatment or prevention of disease, in particular:
53. A product for preventing or treating FVII-related disease or condition or disorder, wherein the product comprises an active substance selected from one or more of the saRNA of any one of embodiments 1-20 or an oligonucleotide modulator of any one of embodiments 21-26, the isolated polynucleotide of any one of embodiments 37-38, the vector of embodiment 39, or the composition of any one of embodiments 43-44.
54. Use of an active substance in the preparation of a product for preventing or treating FVII-related disease or condition or disorder, wherein the active substance is selected from one or more of the saRNA of any one of embodiments 1-20 or an oligonucleotide modulator of any one of embodiments 21-26, the isolated polynucleotide of any one of embodiments 37-38, the vector of embodiment 39, or the composition of any one of embodiments 43-44.
55. A method for preventing or treating FVII-related disease or condition or disorder, wherein the method comprises administering an effective amount of an active substance to a subject, wherein the active substance is selected from one or more of the saRNA of any one of embodiments 1-20 or an oligonucleotide modulator of any one of embodiments 21-26, the isolated polynucleotide of any one of embodiments 37-38, the vector of embodiment 39, or the composition of any one of embodiments 43-44.
56. The product of embodiment 53, the use of embodiment 54 or the method of embodiment 55, wherein the subject is a mammal (such as a human), preferably a mammal suffering from or in risk of having a disease or condition or disorder induced by insufficient expression of factor VII (FVII) protein, a FVII gene mutation, low functional FVII levels in blood and/or other diseases or conditions or disorders preventable or treatable by activating/up-regulating FVII level (such as hemophilia with inhibitor).
57. The product of embodiment 53, the use of embodiment 54 or the method of embodiment 55, wherein the active ingredient is administrated to the individual by an administration route selected from one or more of: parenteral infusions, oral administration, intranasal administration, inhaled administration, vaginal administration, and rectal administration.
58. The product of embodiment 53, the use of embodiment 54 or the method of embodiment 55, wherein the active ingredient is administrated to the individual by an administration route selected from one or more of intrathecal, intramuscular, intravenous, intraarterial, intraperitoneal, intravesical, intracerebroventricular, intravitreal and subcutaneous administrations.
59. The product of embodiment 53, the use of embodiment 54 or the method of embodiment 55, wherein the expression of the FVII gene mRNA in the individual is activates/up-regulates by at least 10% as compared to baseline expression of the FVII gene; and/or wherein the level of FVII protein in the individual is increased by at least 10% as compared to baseline level of FVII protein; and/or
   wherein the FVII-related disease or condition or disorder is selected from the group consisting of congenital FVII deficiency (Alexander's Disease), Acquired FVII deficiency (AFVIID), hemophilia (such as hemophilia with inhibitor) and Glanzmann's thrombasthenia (GT).

In some aspects, provided herein are methods for diagnosis, in particular:
60. A method for diagnosing FVII related disease or disorder by detecting FVII protein or FVII regulated protein in the cell of embodiment 40.

In some aspects, provided herein are kits, in particular:
61. A kit for performing the method of embodiment 60, comprising the saRNA of any one of embodiments 1-20 or the oligonucleotide modulator of any one of embodiments 21-26.
62. The kit of embodiment 61, wherein the instruction for use comprising means for administering the saRNA of any one of embodiments 1-20 or the oligonucleotide modulator of any one of embodiments 21-26 to an individual.
63. A kit comprising the saRNA of any one of embodiments 1-20 or the oligonucleotide modulator of any one of embodiments 21-26, the isolated polynucleotide of any one of embodiments 37-38, the vector of embodiment 39, or the composition of any one of embodiments 43-44 in a labeled package and the label on package indicates that the saRNA, the isolated polynucleotide, the vector or the composition can be used in preventing or treating a disease or condition or disorder induced by insufficient expression of factor VII (FVII), or against hemophilia.
64. A kit for detecting FVII protein or FVII regulated protein in the cell of embodiment 40.

In some aspects, provided herein are methods for obtaining saRNAs, in particular:
65. A Method for obtaining a saRNA capable of up-regulating the expression of FVII gene by at least 10% as compared to baseline expression of FVII gene, wherein the method comprises:
(a) synthesizing a saRNA comprising a sense strand and an antisense strand, wherein each of the sense strand or the antisense strand comprises a consecutive oligonucleotide sequence of 16 to 35 consecutive nucleotides, wherein the consecutive oligonucleotide sequence independently has at least 75%, at least 80%, at least 85%, at least 90%, at least 95%, at least 98% or 100% homology or complementarity to an equal length consecutive fragment of SEQ ID NO: 1437, and wherein the consecutive oligonucleotide sequence has (1) GC content between 40% and 70%; (2) no more than 5 consecutive identical nucleotides; (3) no more than 3 dinucleotide or trinucleotide repeats; and
(b) determining the capacity of the saRNA in up-regulating the expression of FVII gene.
66. A Method for obtaining a saRNA capable of up-regulating the expression of FVII gene by at least 10% as compared to baseline expression of FVII gene, wherein the method comprises:
(a) synthesizing a saRNA targeting a consecutive fragment of SEQ ID NO: 1437, and wherein the saRNA has (1) GC content between 40% and 70%; (2) no more than 5 consecutive identical nucleotides; (3) no more than 3 dinucleotide or trinucleotide repeats; and
(b) determining the capacity of the saRNA in up-regulating the expression of FVII gene.
67. The method of embodiment 65 or 66, wherein the consecutive fragment of SEQ ID NO: 1437 is located in a region upstream of the transcription start site (TSS) of FVII gene, and wherein the consecutive fragment of SEQ ID NO: 1437 is located in a region selected from the group consisting of: region −557 to −379; region −346 to −298; region −271 to −91; and, region −96 to −1; and/or
   the consecutive fragment of SEQ ID NO: 1437 is located in a region of SEQ ID NO: 1438, SEQ ID NO: 1439, SEQ ID NO: 1440, or SEQ ID NO: 1441.
68. The method of embodiment 65 or 66, wherein the consecutive fragment of SEQ ID NO: 1437 is selected from SEQ ID NO: 1-286.

In some aspects, provided herein are isolated double-stranded functional RNAs with blunt-ended structures, in particular:
69. A double-stranded functional saRNA molecule, wherein the functional RNA molecule is blunt-ended at both terminals; and/or
   wherein each strand in the functional RNA molecule has a length of about 16 to about 35, about 17 to about 30, about 18 to about 25, or about 19 to about 22 nucleotides.
70. The double-stranded functional RNA molecule of embodiment 69, wherein the functional RNA molecule targets and regulates FVII gene expression.
71. An oligonucleotide agent comprising one or more saRNA according to any one of claims 69-70.
72. The oligonucleotide agent of embodiment 71, further comprising one or more moieties or components conjugated with said agent(s).
73. The oligonucleotide agent of embodiment 72, wherein the sense strand and/or the antisense strand of the functional saRNA is conjugated to one or more conjugation moieties selected from the group consisting of a lipid, a fatty acid (such as fatty acid comprising a carbon chain with 4-30, 12-24, 16-22 carbon atoms), a fluorophore, a ligand, a saccharide, a peptide, and an antibody.
74. The oligonucleotide agent of embodiment 73, wherein the conjugation moiety is independently selected from a lipid, a cell-penetrating peptide, a polyethylene glycol, an alkaloid, a tryptamine, a benzimidazole, a quinolone, an amino acid, a cholesterol, a glucose, a N-acetylgalactosamine, and any combinations thereof.
75. The oligonucleotide agent of embodiment 74, wherein the conjugation moieties is independently selected from S9, tC2, tC2x6, C5x5, and any combinations thereof

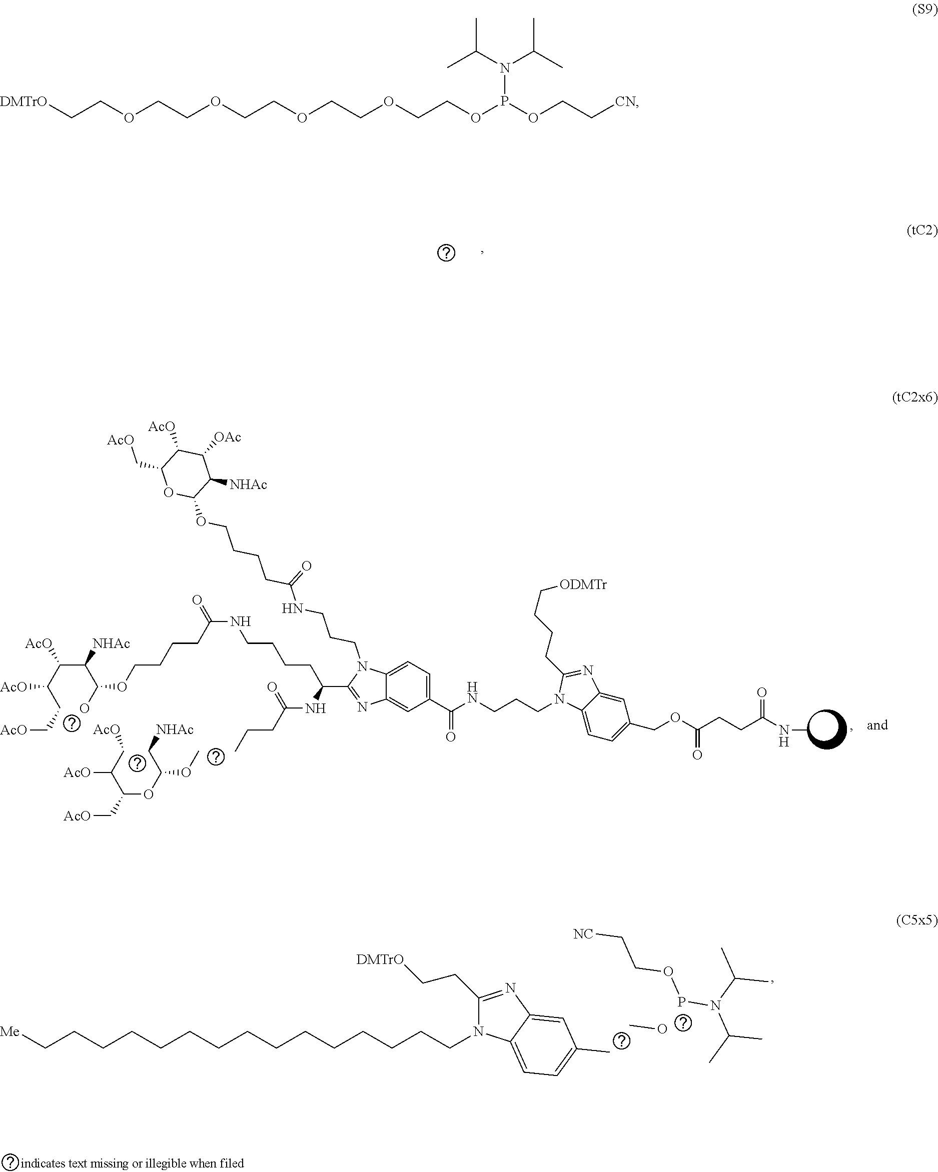

wherein [symbol] represents a support material.

While specific embodiments of the active substances (such as the saRNAs), products, compositions and methods herein have been discussed, many variations of the invention will become apparent to those skilled in the art upon review of this specification and the claims. The full scope of the invention should be determined by reference to the claims, along with their full scope of equivalents, and the specification, along with such variations.

### EXAMPLES

The present application will be further illustrated with reference to specific examples and drawings below. It should be understood that these examples are merely intended to illustrate the present application rather than limit the scope of the present application. In the following examples, study methods without specific conditions were generally in accordance with conventional conditions, such as conditions described in Sambrook, et al., Molecular Cloning: Laboratory Manual (New York: Cold Spring Harbor Laboratory Press, 1989), or conditions recommended by the manufacturer.

### Materials and Methods

saRNA Synthesis

### (1) Single Strand Synthesis

Single strand oligonucleotide was synthesized on a K&A DNA synthesizer (K&A Laborgeraete GbR, chaafheim, Germany) by a solid phase synthesis technique.

The starting material was universal solid support or special solid support commercially available or synthesis as disclosure in previous context. In general, phosphoramidite monomers including various linkers and conjugates (0.1M in acetonitrile or dichloromethane), were added sequentially onto a solid support in the DNA synthesizer to generate the desired full-length oligonucleotides.

Amidite addition: each cycle of amidite addition consisted of four chemical reactions including detritylation, coupling, oxidation/thiolation and capping. In the first step, the detritylation was performed by using 3% dichloroacetic acid (DCA) in DCM for 45 seconds. In the second step, phosphoramidite coupling was conducted for 6 minutes for all amidites by 12 eq. In the third step, oxidation was performed by using 0.02 M iodine in THF:pyridine:water (70:20:10, v/v/v) for 1 minute; if phosphorothioate modification was needed then replace oxidation by thiolation which was carried out with 0.1 M solution of xanthane hydride in pyridine:ACN (50:50, v/v) for 3 minutes. In the fourth step, the capping was performed by using a THF:acetic anhydride:pyridine (80:10:10, v/v/v) (CAP A) and N-methylimidazole:THF (10:90, v/v), (CAP B) for 20 seconds. The cycles of four chemical reactions were depended by the length of single of oligonucleotide.

Deprotection I (Nucleobase Deprotection): after completion of the synthesis, the solid support was transferred to a screw-cap microcentrifuge tube. For a 1 μmol synthesis scale, 1 ml of a mixture of methylamine and ammonium hydroxide was added. The tube containing the solid support was then heated in an oven at 60° C. to 65° C. for 15 min and then allowed to cool to room temperature. The cleavage solution was collected and evaporated to dryness in a speedvac to provide crude single strand of oligonucleotide.

Deprotection II (Removal of 2′-TBDMS Group): if the crude RNA oligonucleotide, still carrying the 2′-TBDMS groups, then dissolved it in 0.1 ml of DMSO. After adding 1 ml of triethylamine trihydrofluoride, the tube was capped, and the mixture was shaken vigorously to ensure complete dissolution and then heated in an oven at 65° C. for 15 minutes. The tube was removed from the oven and cooled down to room temperature. The solution containing the completely desilylated oligonucleotide was cooled on dry ice. Two ml of ice-cold n-butanol (−20° C.) were carefully added in 0.5 ml portions to precipitate the oligonucleotides. The precipitate was filtered, washed with 1 ml ice-cold n-butanol, and subsequently dissolved in 0.01 M Tris(hydroxymethyl)aminomethanol hydrochloride buffer.

### (2) Single Strand Purification

The purification of oligonucleotides was performed on an AKTA explorer 10 equipped with a Source 15Q 4.6/100 PE column using the following conditions: buffer A: (10 mM Tris-HCl, 1 mM EDTA, pH 7.5), B: (10 mM Tris-HCl, 1 mM EDTA, 2M NaCl, pH 7.5), gradient: 10% B to 60% B in 25 min, flow rate: 1 ml/min. The pure oligonucleotides were collected and desalting by a HiPrep 26/10 Desalting column.

### (3) Annealing to Form Duplex

For duplex, after the generation of desalted purified single strand solutions, sense strand and antisense strand were mixed by equal volumes at equimolar concentration in the tube. The tube was placed in a heat block at 95° C. for 5 min and then cooled to room temperature. Then, the thus obtained duplex were subsequently lyophilized to powder.

### Conjugation Moiety

The conjugation moieties can be synthesized via procedures known in the art, for example WO2024002046A1 is fully incorporated herein for synthetic process of tC2, tC2x6, C5x5.

### Cell Culture and Treatment

Human hepatocarcinoma Huh-7 (JCRB0403, Cobioer Biosciences CO.LTD, China) cells and HepG2 cells (SCSP-510, National collection of authenticated cell cultures, China) were cultured at 37° C. with 5% CO₂ in modified DMEM medium (Gibco, Thermo Fisher Scientific, Carlsbad, CA) supplemented with 10% bovine calf serum (Sigma-Aldrich) and 1% penicillin/streptomycin (Gibco). saRNAs were individually transfected into the Huh-7 cells and HepG2 cells in each well at a final concentration of 10, 25 nM, or at indicated concentrations (i.e., 0.1, 0.39, 1.56, 6.25, 25, 100 and 400 nM) with RNAiMAX (Invitrogen, Carlsbad, CA) by following the reverse transfection protocol, respectively. Cells were transfected in the absence of an oligonucleotide as Mock treatment. dsCon2, dsCon2M6v and dsCon2M3v served as a non-targeting duplex control, respectively. RD-13516 was a duplex siRNA for FVII gene and transfected as a silencing siRNA control. RD-15120 was a chemically modified siRNA for FVII gene and transfected as a silencing siRNA control.

### RNA Isolation and Reverse Transcription-Quantitative Polymerase Chain Reaction (RT-iPCR)

### (1) RNA Isolation and One-Step RT-qPCR

At the end of transfection, medium was discarded, and cells were washed once with 150 μL of PBS per well. After discarding the PBS, 100 μL of cell lysis buffer (Power SYBR© Green Cells-to-Ct™ Kit, Life Technologies) was added into each well and the mixture was incubated at room temperature for 5 min. 0.5 μL of the cell lysis was taken from each well and analyzed by RT-qPCR using One Step TB Green© PrimeScrip™ RT-PCR kit II (Takara, RR086A, Shlga, Japan) in a Roche Lightcycler 480 real-time PCR machine (Roche, ref: 4729749001, US). PCR reaction was prepared using Bravo Automated Liquid Handling Platform (Agilent, US). Each transfection sample was amplified in triplicates. PCR reaction mixture was shown in Table 2.

TABLE 2
Composition of the PCR reaction mixture

| Reagent | Volume (μL) |
|---|---|
| 2 × One Step TB Green RT-PCR buffer 4 | 2.5 |
| PrimeScript ™ 1 step enzyme Mix 2 | 0.2 |
| Forward and Reverse Primers Mix (5 μM) | 0.4 |
| dH₂O without RNase | 1.6 |
| Crude lysate (RNA) | 0.4 |
| Total volume | 5.1 |

The reaction conditions were as follows: reverse transcription reaction (stage 1): 42° C. for 5 min, 95° C. for 10 sec; PCR reaction (stage 2): 95° C. for 5 sec, 59° C. for 20 see, 72° C. for 10 sec, 40 cycles of amplification; and melting curve (stage 3). Human FVII gene was amplified as a target gene. Human reference genes (PGK1 and SDHA) were also amplified and their geometric means were used as an internal control for RNA loading. Primer sequences are listed in Table 3.

TABLE 3
Primer sequences for RT-qPCR assay

| | | SEQ ID | | Product |
|---|---|---|---|---|
| Primer | Gene | NO | Sequence (5′-3′) | size (bp) |
| FVII F | Human | 1431 | GGAACTGTGAGACGCACAAG | 169 |
| FVII R | FVII | 1432 | CATGGATATTCAACTGTGGGTG | |
| PGK1 F | Human | 1433 | CTCAACAACATGGAGATTGGCA | 120 |
| PGK1 R | PGK1 | 1434 | GACAAAGTCAACAGGCAAGGT | |
| SDHA F | Human | 1435 | AGAACATCGGAACTGCGACT | 155 |
| SDHA R | SDHA | 1436 | CAGACCATTCCCCGGTCG | |

### (2) RNA Isolation and Two-Step RT-qPCR

For quantifying mRNA expression in cells, total cellular RNA was isolated from treated cells using a RNeasy Plus Mini kit (Qiagen, Hilden, Germany) according to its manual. The resultant RNA (~1 μg) was reverse transcribed into cDNA by using a PrimeScript™ RT reagent kit with gDNA Eraser (Takara, RR047A, Shlga, Japan). The resultant cDNA was amplified in a Roche LightCycler 480 Multiwell Plate 384 (Roche, ref: 4729749001, US) using TB Green® Premix Ex Taq™ II (Takara, RR820A, Shlga, Japan) reagents and primers specifically for amplified target genes of interest.

Reaction conditions were as follows: reverse transcription reaction (stage 1): 42° C. for 5 min, 95° C. for 10 sec; PCR reaction (stage 2): 95° C. for 5 sec, 60° C. for 30 see, 72° C. for 10 sec, 40 cycles of amplification; and melting curve (stage 3). PCR reaction conditions were shown in Table 4 and Table 5.

TABLE 4
RT reaction

| Reaction-1 (Takara, RR047A) | Volume (μL) |
|---|---|
| 5 × gDNA Eraser Buffer | 2 |
| gDNA Eraser | 1 |
| Total RNA (1 μg) + RNase Free dH₂O | 7 |
| Total Volume | 10 |

| 42° C. 5 min, store at 4° C. | |
|---|---|
| Reaction-2 (Takara, RR047A) | Volume (μL) |
| 5 × PrimeScript Buffer2 | 4 |
| PrimeScript RT Enzyme Mix I | 1 |
| RT Prime Mix | 1 |
| RNase free dH₂O | 4 |
| Reaction-1 | 10 |
| Total Volume | 20 |

37° C. 15 min, 85° C. 5 sec, store at 4° C.

TABLE 5
RT-qPCR reaction

| Reagent (Takara, RR820A) | Volume (μL) |
|---|---|
| SYBR Premix Ex Taq II (2×) | 5 |
| PCR Primer (forward + reverse) 5 μM | 1 |
| cDNA (RT product) | 4 |
| Total | 10 |

To calculate the relative expression level (Eᵣₑₗ) of FVII (target gene) mRNA in a saRNA-transfected sample relative to control treatment (Mock), the Ct values of the target gene and the two internal reference genes were substituted into Formula 1,

$${E}_{rel} = {2}^{( CtTm - CtTs )} / ( {( {2}^{CtR 1 m - CtR 1 s )} * {2}^{( CtR 2 m - CtR 2 s )} )}^{( 1 / 2 )} )$$

wherein CtTₘ was the Ct value of the target gene from the mock-treated sample; CtTₛ was the Ct value of the target gene from the saRNA-treated sample; CtR1ₘ was the Ct value of the internal reference gene 1 from the mock-treated sample; CtR1ₛ was the Ct value of the internal reference gene 1 from the saRNA-treated sample; CtR2ₘ was the Ct value of the internal reference gene 2 from the mock-treated sample; and CtR2ₛ was the Ct value of the internal reference gene 2 from the saRNA treated sample.

### Animal Study

The cynomolgus macaques (crab-eating monkeys, male and female) were purchased from Kunming Biomed International (KBI). All animal procedures were conducted by certified laboratory personnel following protocols consistent with local and state regulations and approved by the Institutional Animal Care and Use Committee. Formulations for animal treatments were freshly prepared prior to use by dissolving allotments of lyophilized oligonucleotide into saline to create stock solutions for dilution to the intended treatment concentrations. Animals were randomly allocated into study groups based on body weight and sex.

### Coagulation Function Test

To assess the coagulation function in non-human primates cynomolgus macaque (crab-eating monkeys), the monkeys were administered with the CM-saRNA. One group of monkeys were administered with the indicated CM-saRNA via SC injection at day 0 (10 mg/kg) and day 7 (10 mg/kg). Another group of monkeys were administered with siRNA control via SC injection at day 0 (5 mg/kg). Treatment with saline alone in monkeys served as vehicle control. Plasma samples were harvested from monkeys on day 21 and 28 post first dosing. Plasma samples were prepared by collecting 1.8 mL blood per cynomolgus monkey into plastic blood collection tubes with sodium citrate as an anticoagulant, followed by mixing and centrifuging 10 min at 2500 g within 30 minutes of collection. The samples were detected immediately by automatic coagulation analyzer (BCA-700, GeteinBiotech).

### Example 1: Design and Synthesis of saRNAs Targeting Human FVII Promoter

The coding strand sequence of human FVII gene promoter was retrieved from the UCSC genome database (SEQ ID NO: 1437, as shown in Table 6). It consisted of 600 nucleotides ranging from position −1 bp to −600 bp relative to the transcription start site (TSS).

TABLE 6
Putative human FVII promoter sequence (5′-3′) (SEQ ID NO: 1437)

| | |
|---|---|
| −600 | aagactctgt ctcaaacaaa caaaacaaaa caaacaaaaa gacgtaagat |
| −550 | gtggaccgct ggagaatggg ggtgctgcct gcagtcaaaa cggagtgggg |
| −500 | gtgcccagct cagggccaga atgatcctat tcccggcact tctcagtgag |
| −450 | gctctgtggc tcacctaaga aaccagcctc ccttgcaggc aacggcctag |
| −400 | ctggcctggt ctggaggctc tcttcaaata tttacatcca cacccaagat |
| −350 | acagtcttga gatttgactc gcatgattgc tatgggacaa gttttcatct |
| −300 | gcagtttaaa tctgtttccc aacttacatt aggggtttgg aattctagat |
| −250 | cgtatttgaa gtgttggtgc cacacacacc ttaacacctg cacgctggca |
| −200 | acaaaaccgt ccgctctgca gcacagctgg ggtcacctga cctttctcct |
| −150 | gtccccccca cttgagctca gtggctgggc agcaggggat gcatggccac |
| −100 | tggccggcca ggtgcagctc tcagctgggg tgttcagagg acgcctgtgt |
| −50 | cctcccctcc cccatccctc tgtcaccctt ggaggcagag aactttgccc |

579 possible target sites in the promoter sequence were identified at 22 nucleotides (nt) in length by performing a simple 1-bp walk within the 600 bp promoter region and 286 of which were selected as targets for saRNAs based on the following criteria: (i) GC content between 40% and 70%, (ii) less than 5 consecutive identical nucleotides, and (iii) fewer than 3 dinucleotide or trinucleotide repeats. For the 286 selected target sequences, a total of 858 duplexes were designed (listed in Table 1.1).

### Example 2: High Throughput Screening of saRNAs Targeting Human FVII Promoter

To identify saRNAs capable of up-regulating FVII mRNA expression, Huh-7 cells were transfected with each of the aforementioned saRNAs at 25 nM for 72 hours followed by gene expression analysis via one-step RT-qPCR. A non-targeting duplex (dsCon2) served as a non-targeting control, while a siRNA (i.e., RD-13516) targeting human FVII transcript was used as a transfection control to monitor knockdown via RNA interference (RNAi).

Results showed that 133 (15.5%), 187 (21.8%), and 84 (9.8%) out of 858 tested saRNAs have high (≥1.5 fold), moderate (1.2~1.5 fold), and mild activation (1.1~1.2 fold) on FVII expression, respectively. Results grouped by high, moderate, and mild activation are summarized in Table 7.

TABLE 7
Summary of saRNA activity in inducing FVII
mRNA expression screened in Huh-7 cells

| | log₂ fold change (arithmetic | Number of | Percentage |
|---|---|---|---|
| saRNA activity | fold) in FVII expression | saRNAs | (%) |
| High activation | ≥0.49 (1.50)~≤1.60 (3.03) | 133 | 15.5 |
| Moderate | ≥0.26 (1.20)~<0.49 (1.50) | 187 | 21.8 |
| activation | | | |
| Mild activation | ≥0.13 (1.10)~<0.26 (1.20) | 84 | 9.8 |

Relative changes of FVII expression caused by saRNA treatments are also summarized in Table 1.2 and plotted in FIG. 1.

Sorting all saRNAs by their target site location on human FVII promoter reveals clustering of functional saRNAs in discrete regions or saRNA “hotspot regions” in which target sequences for functional saRNAs are enriched (FIG. 2). They are regions −557 to −379 (H1), −346 to −298 (H2), −271 to −91 (H3) and −96 to −1 (H4) relative to the TSS (FIG. 2). The corresponding DNA sequence for each hotspot region is listed in Table 8.

TABLE 8
Human FVII saRNA hotspot regions and their sequences

| | Hotspot region sequences on FVII promoter | SEQ | Size |
|---|---|---|---|
| Hotspot region | (5′-3′) | ID NO | (nt) |
| H1 | gtaagatgtggaccgctggagaatgggggtgctgcctg | 1438 | 179 |
| (−557 to −379) | cagtcaaaacggagtgggggtgcccagctcagggccag | | |
| | aatgatcctattcccggcacttctcagtgaggctctgt | | |
| | ggctcacctaagaaaccagcctcccttgcaggcaacgg | | |
| | cctagctggcctggtctggaggctctc | | |
| H2 | tcttgagatttgactcgcatgattgctatgggacaagt | 1439 | 49 |
| (−346 to −298) | tttcatctgca | | |
| H3 | taggggtttggaattctagatcgtatttgaagtgttgg | 1440 | 181 |
| (−271 to −91) | tgccacacacaccttaacacctgcacgctggcaacaaa | | |
| | accgtccgctctgcagcacagctggggtcacctgacct | | |
| | ttctcctgtcccccccacttgagctcagtggctgggca | | |
| | gcaggggatgcatggccactggccggcca | | |
| H4 | cggccaggtgcagctctcagctggggtgttcagaggac | 1441 | 96 |
| (−96 to −1) | gcctgtgtcctcccctcccccatccctctgtcaccctt | | |
| | ggaggcagagaactttgccc | | |

### Example 3: Confirmation of Screen Results and Dose-Dependent Induction of FVII mRNA by saRNAs in Huh-7 Cells

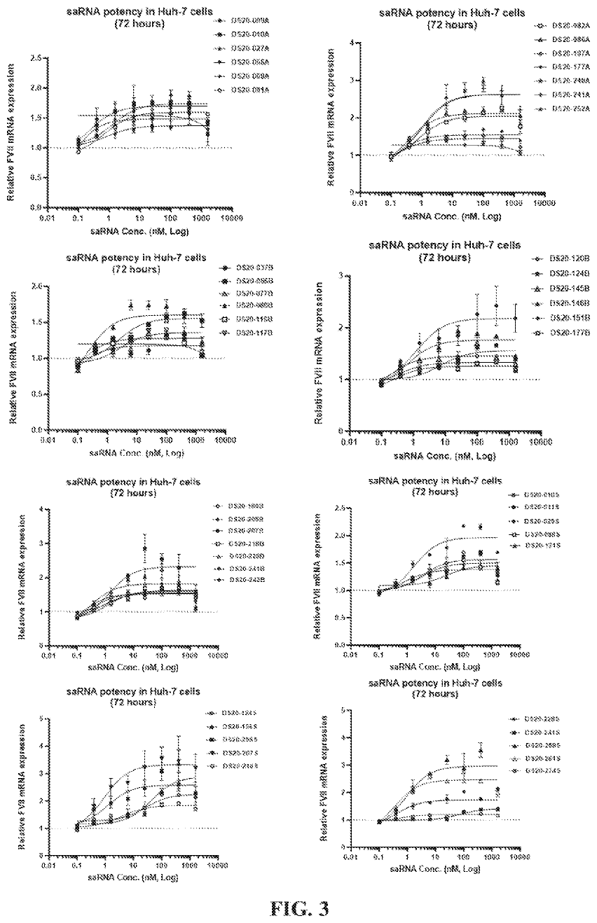

B To confirm the screening results, 47 functional FVII saRNAs were transfected into Huh-7 cells at 7 indicated concentrations (i.e., 0.1, 0.39, 1.56, 6.25, 25, 100 and 400 nM) for 72 hours to generate dose response curve via RT-qPCR (FIG. 3). EC₅₀ values, as well as Eₘₐₓ levels, can be extrapolated to define potency in context to maximal activity for each of the tested saRNAs that demonstrated dose-dependent induction of FVII mRNA (see Table 9).

TABLE 9
FVII mRNA levels caused by saRNAs in Huh-7 cells

| | FVII mRNA levels (fold change) | | | | | | | | | | | | | |
|---|---|---|---|---|---|---|---|---|---|---|---|---|---|---|
| | 0.1 nM | | 0.39 nM | | 1.56 nM | | 6.25 nM | | 25 nM | | 100 nM | | 400 nM | |
| saRNA name | Mean | SEM | Mean | SEM | Mean | SEM | Mean | SEM | Mean | SEM | Mean | SEM | Mean | SEM |
| DS20-207B | 0.85 | 0.004 | 1.15 | 0.004 | 1.43 | 0.025 | 2.05 | 0.042 | 2.85 | 0.290 | 2.56 | 0.110 | 2.29 | 0.283 |
| DS20-151B | 0.93 | 0.014 | 1.35 | 0.032 | 1.71 | 0.127 | 1.79 | 0.134 | 1.85 | 0.134 | 2.26 | 0.276 | 2.43 | 0.272 |
| DS20-228B | 0.91 | 0.046 | 1.24 | 0.060 | 1.58 | 0.088 | 1.95 | 0.113 | 2.03 | 0.170 | 2.24 | 0.170 | 1.76 | 0.078 |
| DS20-146B | 0.92 | 0.007 | 1.28 | 0.007 | 1.40 | 0.028 | 1.63 | 0.060 | 1.94 | 0.028 | 1.89 | 0.028 | 1.84 | 0.007 |
| DS20-086B | 0.91 | 0.071 | 1.22 | 0.011 | 1.43 | 0.021 | 1.74 | 0.049 | 1.74 | 0.042 | 1.73 | 0.067 | 1.59 | 0.074 |
| DS20-242B | 0.97 | 0.014 | 1.17 | 0.004 | 1.29 | 0.021 | 1.38 | 0.042 | 1.63 | 0.028 | 1.73 | 0.014 | 1.67 | 0.039 |
| DS20-188B | 0.89 | 0.011 | 1.12 | 0.078 | 1.43 | 0.032 | 1.55 | 0.067 | 1.42 | 0.039 | 1.58 | 0.078 | 1.70 | 0.085 |
| DS20-145B | 0.97 | 0.014 | 1.23 | 0.035 | 1.37 | 0.018 | 1.40 | 0.103 | 1.43 | 0.081 | 1.71 | 0.074 | 1.42 | 0.018 |
| DS20-218B | 0.82 | 0.004 | 1.05 | 0.007 | 1.25 | 0.060 | 1.40 | 0.053 | 1.69 | 0.028 | 1.69 | 0.032 | 1.67 | 0.099 |
| DS20-241B | 0.87 | 0.011 | 1.10 | 0.032 | 1.29 | 0.021 | 1.38 | 0.028 | 1.62 | 0.095 | 1.80 | 0.113 | 1.57 | 0.004 |
| DS20-205B | 0.85 | 0.014 | 1.22 | 0.071 | 1.44 | 0.028 | 1.57 | 0.004 | 1.51 | 0.071 | 1.62 | 0.025 | 1.78 | 0.209 |
| DS20-055B | 0.90 | 0.039 | 1.13 | 0.011 | 1.27 | 0.028 | 1.27 | 0.035 | 1.47 | 0.007 | 1.58 | 0.021 | 1.62 | 0.049 |
| DS20-124B | 0.91 | 0.039 | 1.11 | 0.011 | 1.13 | 0.018 | 1.23 | 0.028 | 1.38 | 0.060 | 1.62 | 0.049 | 1.66 | 0.014 |
| DS20-120B | 0.97 | 0.039 | 1.16 | 0.057 | 1.21 | 0.049 | 1.27 | 0.039 | 1.32 | 0.060 | 1.46 | 0.004 | 1.41 | 0.049 |
| DS20-117B | 0.91 | 0.032 | 1.14 | 0.032 | 1.19 | 0.042 | 1.29 | 0.046 | 1.38 | 0.007 | 1.40 | 0.004 | 1.26 | 0.014 |
| DS20-077B | 0.84 | 0.018 | 1.12 | 0.032 | 1.10 | 0.053 | 1.13 | 0.078 | 1.35 | 0.049 | 1.53 | 0.014 | 1.36 | 0.060 |
| DS20-116B | 0.96 | 0.011 | 1.14 | 0.000 | 1.21 | 0.007 | 1.26 | 0.039 | 1.30 | 0.018 | 1.30 | 0.028 | 1.22 | 0.032 |
| DS20-177B | 0.90 | 0.032 | 1.07 | 0.028 | 1.19 | 0.032 | 1.25 | 0.014 | 1.21 | 0.032 | 1.31 | 0.032 | 1.34 | 0.000 |
| DS20-037B | 0.88 | 0.000 | 1.07 | 0.007 | 1.13 | 0.018 | 1.07 | 0.032 | 1.11 | 0.042 | 1.33 | 0.039 | 1.33 | 0.064 |
| DS20-252A | 0.94 | 0.028 | 1.33 | 0.039 | 1.83 | 0.067 | 2.56 | 0.233 | 2.72 | 0.124 | 2.99 | 0.064 | 2.61 | 0.180 |
| DS20-241A | 0.97 | 0.025 | 1.36 | 0.042 | 1.87 | 0.025 | 2.33 | 0.032 | 2.75 | 0.134 | 2.78 | 0.064 | 2.72 | 0.035 |
| DS20-086A | 0.96 | 0.085 | 1.46 | 0.032 | 1.76 | 0.046 | 2.12 | 0.042 | 2.19 | 0.018 | 2.20 | 0.067 | 2.21 | 0.021 |
| DS20-082A | 0.96 | 0.042 | 1.28 | 0.018 | 1.63 | 0.032 | 1.88 | 0.021 | 2.00 | 0.064 | 2.13 | 0.028 | 2.26 | 0.028 |
| DS20-010A | 1.14 | 0.046 | 1.38 | 0.039 | 1.61 | 0.095 | 1.75 | 0.067 | 1.77 | 0.081 | 1.77 | 0.011 | 1.75 | 0.085 |
| DS20-027A | 1.08 | 0.011 | 1.22 | 0.018 | 1.40 | 0.014 | 1.62 | 0.021 | 1.74 | 0.000 | 1.90 | 0.018 | 1.88 | 0.042 |
| DS20-009A | 1.09 | 0.021 | 1.48 | 0.134 | 1.63 | 0.103 | 1.71 | 0.240 | 1.49 | 0.103 | 1.71 | 0.184 | 1.66 | 0.120 |
| DS20-177A | 0.91 | 0.049 | 1.26 | 0.011 | 1.48 | 0.053 | 1.48 | 0.035 | 1.48 | 0.014 | 1.69 | 0.007 | 1.64 | 0.028 |
| DS20-081A | 0.93 | 0.014 | 1.20 | 0.004 | 1.35 | 0.032 | 1.47 | 0.032 | 1.55 | 0.032 | 1.58 | 0.004 | 1.72 | 0.035 |
| DS20-069A | 1.01 | 0.025 | 1.31 | 0.035 | 1.48 | 0.067 | 1.53 | 0.095 | 1.38 | 0.064 | 1.67 | 0.216 | 1.60 | 0.028 |
| DS20-107A | 0.97 | 0.014 | 1.21 | 0.025 | 1.35 | 0.004 | 1.46 | 0.060 | 1.53 | 0.085 | 1.52 | 0.113 | 1.48 | 0.042 |
| DS20-055A | 1.02 | 0.021 | 1.20 | 0.025 | 1.22 | 0.042 | 1.29 | 0.028 | 1.32 | 0.042 | 1.33 | 0.011 | 1.56 | 0.117 |
| DS20-240A | 1.01 | 0.053 | 1,23 | 0.032 | 1.40 | 0.032 | 1.38 | 0.000 | 1.28 | 0.018 | 1.31 | 0.042 | 1.28 | 0.067 |
| DS20-207S | 1.07 | 0.049 | 1.80 | 0.205 | 2.50 | 0.226 | 2.66 | 0.187 | 3.22 | 0.435 | 3.56 | 0.290 | 3.46 | 0.293 |
| DS20-259S | 0.93 | 0.018 | 1.38 | 0.035 | 2.09 | 0.078 | 2.70 | 0.007 | 3.20 | 0.120 | 2.90 | 0.378 | 3.55 | 0.184 |
| DS20-205S | 1.05 | 0.081 | 1.69 | 0.148 | 1.92 | 0.127 | 2.08 | 0.177 | 2.47 | 0.212 | 3.08 | 0.237 | 2.61 | 0.110 |
| DS20-261S | 0.96 | 0.011 | 1.43 | 0.000 | 2.21 | 0.177 | 2.61 | 0.074 | 2.42 | 0.032 | 2.76 | 0.000 | 2.42 | 0.000 |
| DS20-124S | 1.08 | 0.007 | 1.34 | 0.011 | 1.47 | 0.014 | 1.49 | 0.042 | 1.57 | 0.007 | 2.24 | 0.039 | 2.22 | 0.035 |
| DS20-156S | 1.04 | 0.007 | 1.20 | 0.039 | 1.27 | 0.018 | 1.57 | 0.103 | 1.52 | 0.035 | 2.36 | 0.074 | 3.37 | 0.711 |
| DS20-029S | 0.94 | 0.025 | 1.11 | 0.007 | 1.48 | 0.042 | 1.65 | 0.007 | 1.78 | 0.127 | 2.18 | 0.004 | 2.16 | 0.035 |
| DS20-228S | 0.91 | 0.025 | 1.23 | 0.049 | 1.51 | 0.071 | 1.64 | 0.035 | 1.77 | 0.110 | 2.03 | 0.025 | 1.77 | 0.032 |
| DS20-218S | 0.96 | 0.018 | 1.21 | 0.057 | 1.33 | 0.067 | 1.44 | 0.035 | 1.76 | 0.152 | 1.86 | 0.057 | 1.92 | 0.035 |
| DS20-010S | 0.96 | 0.004 | 1.09 | 0.004 | 1.21 | 0.025 | 1.29 | 0.138 | 1.50 | 0.057 | 1.70 | 0.011 | 1.67 | 0.028 |
| DS20-011S | 0.96 | 0.025 | 1.08 | 0.021 | 1.21 | 0.007 | 1.30 | 0.032 | 1.33 | 0.064 | 1.62 | 0.014 | 1.70 | 0.025 |
| DS20-088S | 0.99 | 0.053 | 1.12 | 0.060 | 1.18 | 0.049 | 1.37 | 0.074 | 1.37 | 0.060 | 1.55 | 0.057 | 1.43 | 0.018 |
| DS20-121S | 0.99 | 0.004 | 1.12 | 0.028 | 1.22 | 0.028 | 1.14 | 0.060 | 1.25 | 0.004 | 1.42 | 0.032 | 1.46 | 0.060 |
| DS20-274S | 0.97 | 0.004 | 1.07 | 0.007 | 1.12 | 0.000 | 1.18 | 0.011 | 1.13 | 0.011 | 1.30 | 0.000 | 1.22 | 0.000 |
| DS20-241S | 0.95 | 0.028 | 1.02 | 0.035 | 1.03 | 0.018 | 1.02 | 0.021 | 1.02 | 0.039 | 1.29 | 0.014 | 1.38 | 0.014 |

Note:
SEM represents Standard Error of the Mean.

### Example 4: In Vitro Activation of FVII saRNA and Chemically Modified saRNAs (CM-saRNAs) in HepG2 and Huh-7 Cells

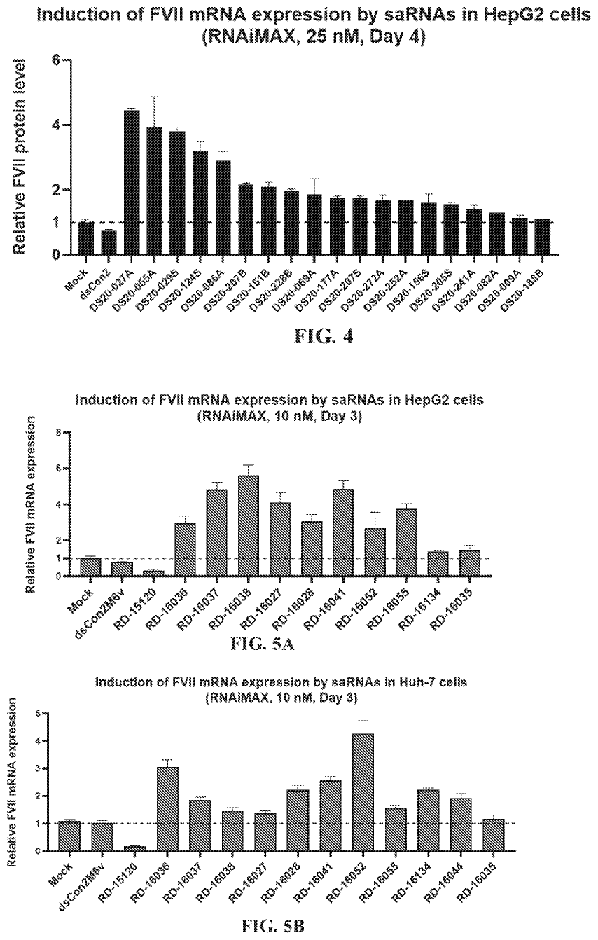

To assess the activation of FVII saRNAs on the expression of FVII protein, 19 indicated saRNAs (i.e., DS20-027A, DS20-055A, DS2-029S, DS20-124S, DS20-086A, DS2-207B, DS20-151B, DS20-228B, DS20-069A, DS20-177A, DS20-207S, DS20-272A, DS20-252A, DS20-156S, DS20-205S, DS20-241A, DS20-082A, DS20-009A and DS20-188B) were individually transfected into HepG2 cells at 25 nM for 4 days. FVII protein levels were detected by western blotting using an antibody against human FVII protein. FIG. 4 showed the FVII protein levels following saRNA treatments in HepG2 cells. Table 10 summarizes the FVIJ protein levels following saRNA treatments in HepG2 cells.

TABLE 10
FVII protein levels following FVII
saRNA treatments in HepG2 cells

| | |
|---|---|
| saRNA | FVII protein level (25 nM) |

| name | Mean | SEM |
|---|---|---|
| Mock | 1.00 | 0.020 |
| dsCon2 | 0.73 | 0.019 |
| DS20-027A | 4.45 | 0.035 |
| DS20-055A | 3.95 | 0.460 |
| DS20-029S | 3.80 | 0.071 |
| DS20-124S | 3.20 | 0.141 |
| DS20-086A | 2.90 | 0.141 |
| DS20-207B | 2.15 | 0.035 |
| DS20-151B | 2.10 | 0.071 |
| DS20-228B | 1.95 | 0.035 |
| DS20-069A | 1.85 | 0.247 |
| DS20-177A | 1.75 | 0.035 |
| DS20-207S | 1.75 | 0.035 |
| DS20-272A | 1.70 | 0.071 |
| DS20-252A | 1.70 | 0.000 |
| DS20-156S | 1.60 | 0.141 |
| DS20-205S | 1.55 | 0.035 |
| DS20-241A | 1.40 | 0.071 |
| DS20-082A | 1.30 | 0.000 |
| DS20-009A | 1.15 | 0.035 |
| DS20-188B | 1.10 | 0.000 |

Note:
SEM represents Standard Error of the Mean.

To assess the activation of FVII CM-saRNAs on the expression of FVII mRNA, 10 indicated FVII CM-saRNAs (i.e., RD-16036, RD-16037, RD-16038, RD-16027, RD-16028, RD-16041, RD-16052, RD-16055, RD-16134 and RD-16035) (see Table 11) were transfected into HepG2 cells at 10 nM for 3 days. 11 indicated FVII CM-saRNAs (i.e., RD-16036, RD-16037, RD-16038, RD-16027, RD-16028, RD-16041, RD-16052, RD-16055, RD-16134, RD-16044 and RD-16035) (see Table 11) were transfected into Huh-7 cells at 25 nM for 3 days. RD-15120 was a chemically modified siRNA for FVII gene and transfected as a silencing siRNA control. FVII mRNA levels were quantified by two step RT-qPCR. FIG. 5A-5B showed the FVII mRNA levels following CM-saRNA treatments in HepG2 and Huh-7 cells. Table 12 summarizes the FVII mRNA levels following CM-saRNA treatments in HepG2 and Huh-7 cells.

TABLE 11
Oligonucleotide sequences and compositions

| Oligonucleotide | | SEQ | | Size |
|---|---|---|---|---|
| name | Strand | ID NO | Sequence (5′-3′) | (nt) |
| RD-16012 | sense | 1442 | mG*fG*mUfGmCfUmGfCfCfUfGfCmAfGmUfCmAfAmAfA | 20 |
| | antisense | 1443 | VpmU*fU*mUfUmGfAmCfUmGfCmAfGmGfCmAfGmCfAmCf | 22 |
| | | | C*mC*fC | |
| RD-16013 | sense | 1444 | mC*fU*mCfAmGfGmGfCfCfAfGfAmAfUmGfAmUfCmCfU | 20 |
| | antisense | 1445 | VpmA*fG*mGfAmUfCmAfUmUfCmUfGmGfCmCfCmUfGmAf | 22 |
| | | | G*mC*fU | |
| RD-16017 | sense | 1446 | mA*fG*mAfAmAfCmCfAfGfCfCfUmCfCmCfUmUfGmCfA | 20 |
| | antisense | 1447 | VpmU*fG*mCfAmAfGmGfGmAfGmGfCmUfGmGfUmUfUmCf | 22 |
| | | | U*mU*fA | |
| RD-16024 | sense | 1448 | mG*mG*mUmGmCfUmGfCfCfUmGmCmAmGmUmCmAmAm | 20 |
| | | | AmA | |
| | antisense | 1449 | VpmU*fU*mUmUmGfAmCmUmGmCmAfGmGfCmAmGmCmA | 22 |
| | | | mCmC*mC*mC | |
| RD-16027 | sense | 1450 | mG*mC*mUmCmUfGmUfGfGfCmUmCmAmCmCmUmAmAm | 20 |
| | | | GmA | |
| | antisense | 1451 | VpmU*fC*mUmUmAfGmGmUmGmAmGfCmCfAmCmAmGmA | 22 |
| | | | mGmC*mC*mU | |
| RD-16028 | sense | 1452 | mC*mC*mUmAmAfGmAfAfAfCmCmAmGmCmCmUmCmCmC | 20 |
| | | | mU | |
| | antisense | 1453 | VpmA*fG*mGmGmAfGmGmCmUmGmGfUmUfUmCmUmUm | 22 |
| | | | AmGmG*mU*mG | |
| RD-16029 | sense | 1454 | mA*mG*mAmAmAfCmCfAfGfCmCmUmCmCmCmUmUmGm | 20 |
| | | | CmA | |
| | antisense | 1455 | VpmU*fG*mCmAmAfGmGmGmAmGmGfCmUfGmGmUmUm | 22 |
| | | | UmCmU*mU*mA | |
| RD-16035 | sense | 1456 | mA*mG*mGmAmCfGmCfCfUfGmUmGmUmCmCmUmCmCm | 20 |
| | | | CmC | |
| | antisense | 1457 | VpmG*fG*mGmGmAfGmGmAmCmAmCfAmGfGmCmGmUmC | 22 |
| | | | mCmU*mC*mU | |
| RD-16036 | sense | 1458 | mG*mG*mUmGmCfUmGfCfCfUmGmCmAfGmUmCmAmAmA | 20 |
| | | | mA | |
| | antisense | 1459 | VpmU*fU*mUfUmGfAmCfUmGmCmAfGmGfCmAfGmCmAm | 22 |
| | | | CmC*mC*mC | |
| RD-16037 | sense | 1460 | mC*mU*mCmAmGfGmGfCfCfAmGmAmAfUmGmAmUmCmC | 20 |
| | | | mU | |
| | antisense | 1461 | VpmA*fG*mGfAmUfCmAfUmUmCmUfGmGfCmCfCmUmGm | 22 |
| | | | AmG*mC*mU | |
| RD-16038 | sense | 1462 | mC*mA*mCmUmUfCmUfCfAfGmUmGmAfGmGmCmUmCmU | 20 |
| | | | mG | |
| | antisense | 1463 | VpmC*fA*mGfAmGfCmCfUmCmAmCfUmGfAmGfAmAmGm | 22 |
| | | | UmG*mC*mC | |
| RD-16041 | sense | 1464 | mA*mG*mAmAmAfCmCfAfGfCmCmUmCfCmCmUmUmGmC | 20 |
| | | | mA | |
| | antisense | 1465 | VpmU*fG*mCfAmAfGmGfGmAmGmGfCmUfGmGfUmUmUm | 22 |
| | | | CmU*mU*mA | |
| RD-16044 | sense | 1466 | mG*mC*mCmAmGfGmUfGfCfAmGmCmUfCmUmCmAmGmC | 20 |
| | | | mU | |
| | antisense | 1467 | VpmA*fG*mCfUmGfAmGfAmGmCmUfGmCfAmCfCmUmGm | 22 |
| | | | GmC*mC*mG | |
| RD-16052 | sense | 1468 | mCfGmUfAmUfUmUfGfAfAfGmUmGfUfUmGmGmUmGfCmC | 21 |
| | antisense | 1469 | mGfGmCfAmCfCmAfAfCfAfCmUmUfCfAmAmAmUmAfCmG | 21 |
| RD-16055 | sense | 1470 | mCfUmGfGmGmGmUmCfAdCfCmUmGmAfCmCmUmUmUfC | 21 |
| | | | mU | |
| | antisense | 1471 | mAfGmAfAmAmGmGmUfCdAfGmGmUmGfAmCmCmCmCfA | 21 |
| | | | mG | |
| RD-16120 | sense | 1472 | VpmC*fU*mGfGmGfGmUfGmUfUmCfAmGfAmGfGmAfCmGf | 22 |
| | | | C*mC*fU | |
| | antisense | 1473 | mG*fC*mGfUmCfCmUfCfUfGfAfAmCfAmCfCmCfCmAfG | 20 |
| RD-16134 | sense | 1474 | VpmC*fU*mGmGmGfGmUmCmAmCmCfUmGfAmCmCmUmU | 22 |
| | | | mUmC*mU*mC | |
| | antisense | 1475 | mG*mA*mAmAmGfGmUfCfAfGmGmUmGfAmCfCmCfCmAm | 20 |
| | | | G | |
| RD-17272 | sense | 1476 | mC*mU*mCmAmGfGmGfCfCfAmGmAmAfUmGmAmUmCmC | 20 |
| | | | mU-tC2×6 | |
| | antisense | 1461 | VpmA*fG*mGfAmUfCmAfUmUmCmUfGmGfCmCfCmUmGm | 22 |
| | | | AmG*mC*mU | |
| RD-16985 | sense | 1477 | mU*mC*mCmUmGfGmCfAfCfCmAmAmAmUmCmCmCmAm | 20 |
| | | | UmA-tC2x6 | |
| | antisense | 1478 | VpmU*fA*mUmGmGfGmAmUmUmUmGfGmUfGmCmCmAm | 22 |
| | | | GmGmA*mC*mA | |
| RD-15120 | sense | 1479 | mU*mC*mCmUmGfGmCfAfCfCmAmAmAmUmCmCmCmA*m | 20 |
| | | | U*mA | |
| | antisense | 1478 | VpmU*fA*mUmGmGfGmAmUmUmUmGfGmUfGmCmCmAm | 22 |
| | | | GmGmA*mC*mA | |
| RD-13516 | sense | 1480 | CCUGGCACCAAAUCCCAUATT | 21 |
| | antisense | 1481 | UAUGGGAUUUGGUGCCAGGTT | 21 |
| dsCon2 | sense | 1482 | ACUACUGAGUGACAGUAGATT | 21 |
| | antisense | 1483 | UCUACUGUCACUCAGUAGUTT | 21 |
| dsCon2M3v | sense | 1484 | mG*fA*mCfUmAfCmUfGmAfGfUfGmAfCmAfGmUfA*mG*fA | 20 |
| | antisense | 1485 | VpmU*fC*mUfAmCfUmGfUmCfAmCfUmCfAmGfUmAfGmUf | 22 |
| | | | C*mG*fU | |
| dsCon2M6v | sense | 1486 | mG*mA*mCmUmAfCmUfGfAfGmUmGmAmCmAmGmUmA* | 20 |
| | | | mG*mA | |
| | antisense | 1487 | VpmU*fC*mUmAmCfUmGmUmCmAmCfUmCfAmGmUmAmG | 22 |
| | | | mUmC*mG*mU | |

Note:
Uppercase, RNA; *, phosphorothioate (PS) backbone modification; f, 2′-fluoro; m, 2′-O-methyl (2′-OMe); Vp, 5′-(E)-vinylphosphonate; dC, Cytosine deoxyribonucleic acid; dA, Adenosine deoxyribonucleic acid; tC2x6 compound see WO2024002046A1 application.

TABLE 12
FVII mRNA levels following FVII CM-saRNA
treatments in HepG2 and Huh-7 cells

| | | |
|---|---|---|
| | FVII mRNA level in | FVII mRNA level in |
| CM-saRNA | HepG2 cells (10 nM) | Huh-7 cells (10 nM) |

| name | Mean | SEM | Mean | SEM |
|---|---|---|---|---|
| Mock | 1.02 | 0.025 | 1.09 | 0.014 |
| dsCon2M6v | 0.81 | 0.006 | 1.04 | 0.018 |
| RD-15120 | 0.32 | 0.019 | 0.18 | 0.006 |
| RD-16036 | 2.95 | 0.104 | 3.05 | 0.065 |
| RD-16037 | 4.83 | 0.102 | 1.86 | 0.024 |
| RD-16038 | 5.63 | 0.142 | 1.46 | 0.032 |
| RD-16027 | 4.09 | 0.144 | 1.36 | 0.025 |
| RD-16028 | 3.08 | 0.092 | 2.21 | 0.044 |
| RD-16041 | 4.89 | 0.115 | 2.58 | 0.035 |
| RD-16052 | 2.69 | 0.219 | 4.26 | 0.120 |
| RD-16055 | 3.79 | 0.067 | 1.57 | 0.023 |
| RD-16134 | 1.38 | 0.017 | 2.23 | 0.016 |
| RD-16044 | / | / | 1.92 | 0.042 |
| RD-16035 | 1.46 | 0.069 | 1.18 | 0.031 |

Note:
SEM represents Standard Error of the Mean.
/ represents not tested.

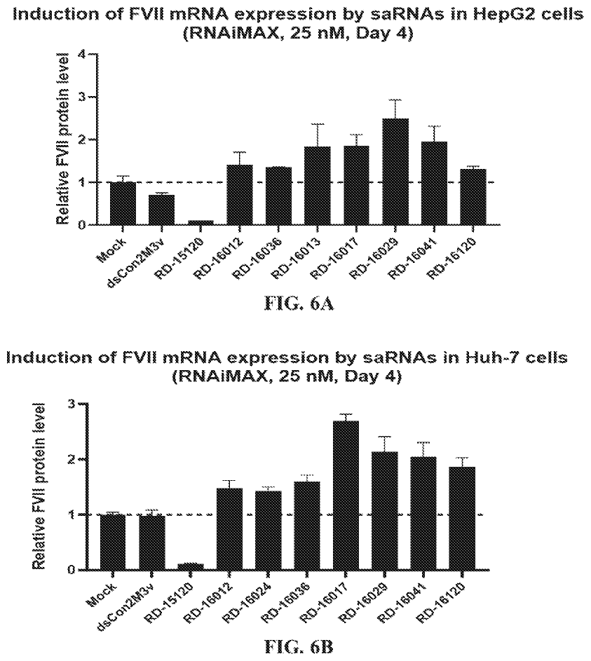

To further assess the activation of FVII CM-saRNAs on the expression of FVII protein, 7 indicated FVII CM-saRNAs (i.e., RD-16012, RD-16036, RD-16013, RD-16017, RD-16029, RD-16041 and RD-16120) were transfected into HepG2 cells at 25 nM for 4 days. 7 indicated FVII CM-saRNAs (i.e., RD-16012, RD-16024, RD-16036, RD-16017, RD-16029, RD-16041 and RD-16120) were transfected into Huh-7 cells at 25 nM for 4 days. RD-15120 was a chemically modified siRNA for FVII gene and transfected as a silencing siRNA control. FVII protein levels were detected by western blotting using an antibody against human FVII protein. As shown in FIG. 6A-6B, all CM-saRNAs induced more than 1.3-fold induction. FVII protein levels following CM-saRNA treatments in HepG2 and Huh-7 cells are summarized in Table 13.

TABLE 13
FVII protein levels following FVII CM-
saRNA treatments in HepG2 and Huh-7 cells

| | | |
|---|---|---|
| | FVII protein level in | FVII protein level in |
| CM-saRNA | HepG2 cells (10 nM) | Huh-7 cells (10 nM) |

| name | Mean | SEM | Mean | SEM |
|---|---|---|---|---|
| Mock | 1.07 | 0.103 | 0.97 | 0.014 |
| dsCon2M3v | 0.71 | 0.025 | 0.97 | 0.057 |
| RD-15120 | 0.11 | 0.000 | 0.12 | 0.004 |
| RD-16012 | 1.42 | 0.145 | 1.49 | 0.067 |
| RD-16024 | / | / | 1.43 | 0.039 |
| RD-16036 | 1.35 | 0.007 | 1.60 | 0.060 |
| RD-16013 | 1.84 | 0.262 | / | / |
| RD-16017 | 1.85 | 0.134 | 2.70 | 0.060 |
| RD-16029 | 2.49 | 0.223 | 2.14 | 0.138 |
| RD-16041 | 1.96 | 0.180 | 2.06 | 0.124 |
| RD-16120 | 1.32 | 0.032 | 1.86 | 0.085 |

Note:
SEM represents Standard Error of the Mean.
/ represents not tested.

### Example 5: Improvement of Coagulation Function in Non-Human Primates Treated by FVII CM-saRNAs

To assess the coagulation function in non-human primates cynomolgus macaque (crab-eating monkeys), the monkeys were administered with the CM-saRNA. One group of monkeys (one male about 4.1~4.9 years old, weighing 3.33~4.68 kg; one female about 4.2~4.8 years old, weighing 2.54~3.09 kg) were administered with the indicated CM-saRNA (i.e., RD-17272) via SC injection at day 0 (10 mg/kg) and day 7 (10 mg/kg). Another group of monkeys (one male about 4.1~4.9 years old, weighing 3.33~4.68 kg; one female about 4.2~4.8 years old, weighing 2.54~3.09 kg) were administered with RD-16985 via SC injection at day 0 (5 mg/kg) to serve as a silencing siRNA control. Treatment with saline alone in monkeys served as vehicle control. Plasma samples were harvested from monkeys on day 21 and 28 post first dosing. Prothrombin time (second, s) of monkey plasmas were detected by automatic coagulation analyzer on day 21 and 28 post first dosing. Thrombin time (second, s) of monkey plasmas were detected by automatic coagulation analyzer on day 28 post first dosing. Prothrombin time and thrombin time were summarized in Table 14.

TABLE 14
Prothrombin time and thrombin time in monkey plasmas.

Prothrombin time (second, s)

| Group (Day 21, n = 2) | Mean | SEM |
|---|---|---|
| Saline | 8.10 | 0.071 |
| RD-16985 | 8.35 | 0.035 |
| RD-17272 | 7.85 | 0.106 |

Prothrombin time (second, s)

| Group (Day 28, n = 2) | Mean | SEM |
|---|---|---|
| Saline | 8.40 | 0.283 |
| RD-16985 | 8.65 | 0.177 |
| RD-17272 | 8.00 | 0.283 |

Thrombin time (second, s)

| Group (Day 28, n = 2) | Mean | SEM |
|---|---|---|
| Saline | 15.45 | 0.106 |
| RD-16985 | 16.30 | 0.141 |
| RD-17272 | 15.25 | 0.389 |

Note:
SEM represents Standard Error of Mean.

In summary, the high throughput screening data revealed a plurality of “hotspot regions” for saRNA activity in the promoter of human FVII gene. Exemplary saRNAs increased expression of both FVII mRNA and FVII protein levels while demonstrating a dose-dependent manner. These results provide evidence that targeted activation of FVII expression via saRNAs is a promising strategy to treat FVII-related disease or condition or disorder, such as congenital FVII deficiency (Alexander's Disease), Acquired FVII deficiency (AFVIID), hemophilia (e.g., hemophilia with inhibitor), Glanzmann's thrombasthenia (GT).

TABLE 1.1
Target sequences and strand compositions of human FVII gene saRNAs

| | SEQ | | SEQ | | SEQ | |
|---|---|---|---|---|---|---|
| saRNA | ID | Target | ID | | ID | Antisense |
| name | NO | sequence (5′-3′) | NO | Sense strand (5′-3′) | NO | strand (5′-3′) |
| DS20-001B | 1 | cgtaagatgtggaccgctggag | 287 | CGUAAGAUGUGGACCGCUGGAG | 859 | CUCCAGCGGUCCACAUCUUACG |
| DS20-002B | 2 | gtaagatgtggaccgctggaga | 288 | GUAAGAUGUGGACCGCUGGAGA | 860 | UCUCCAGCGGUCCACAUCUUAC |
| DS20-003B | 3 | taagatgtggaccgctggagaa | 289 | UAAGAUGUGGACCGCUGGAGAA | 861 | UUCUCCAGCGGUCCACAUCUUA |
| DS20-004B | 4 | aagatgtggaccgctggagaat | 290 | AAGAUGUGGACCGCUGGAGAAU | 862 | AUUCUCCAGCGGUCCACAUCUU |
| DS20-005B | 5 | agatgtggaccgctggagaatg | 291 | AGAUGUGGACCGCUGGAGAAUG | 863 | CAUUCUCCAGCGGUCCACAUCU |
| DS20-006B | 6 | gatgtggaccgctggagaatgg | 292 | GAUGUGGACCGCUGGAGAAUGG | 864 | CCAUUCUCCAGCGGUCCACAUC |
| DS20-007B | 7 | atgtggaccgctggagaatggg | 293 | AUGUGGACCGCUGGAGAAUGGG | 865 | CCCAUUCUCCAGCGGUCCACAU |
| DS20-008B | 8 | tgtggaccgctggagaatgggg | 294 | UGUGGACCGCUGGAGAAUGGGG | 866 | CCCCAUUCUCCAGCGGUCCACA |
| DS20-009B | 9 | ggggtgctgcctgcagtcaaaa | 295 | GGGGUGCUGCCUGCAGUCAAAA | 867 | UUUUGACUGCAGGCAGCACCCC |
| DS20-010B | 10 | gggtgctgcctgcagtcaaaac | 296 | GGGUGCUGCCUGCAGUCAAAAC | 868 | GUUUUGACUGCAGGCAGCACCC |
| DS20-011B | 11 | ggtgctgcctgcagtcaaaacg | 297 | GGUGCUGCCUGCAGUCAAAACG | 869 | CGUUUUGACUGCAGGCAGCACC |
| DS20-012B | 12 | gtgctgcctgcagtcaaaacgg | 298 | GUGCUGCCUGCAGUCAAAACGG | 870 | CCGUUUUGACUGCAGGCAGCAC |
| DS20-013B | 13 | tgctgcctgcagtcaaaacgga | 299 | UGCUGCCUGCAGUCAAAACGGA | 871 | UCCGUUUUGACUGCAGGCAGCA |
| DS20-014B | 14 | gctgcctgcagtcaaaacggag | 300 | GCUGCCUGCAGUCAAAACGGAG | 872 | CUCCGUUUUGACUGCAGGCAGC |
| DS20-015B | 15 | ctgcctgcagtcaaaacggagt | 301 | CUGCCUGCAGUCAAAACGGAGU | 873 | ACUCCGUUUUGACUGCAGGCAG |
| DS20-016B | 16 | tgcctgcagtcaaaacggagtg | 302 | UGCCUGCAGUCAAAACGGAGUG | 874 | CACUCCGUUUUGACUGCAGGCA |
| DS20-017B | 17 | gcctgcagtcaaaacggagtgg | 303 | GCCUGCAGUCAAAACGGAGUGG | 875 | CCACUCCGUUUUGACUGCAGGC |
| DS20-018B | 18 | cctgcagtcaaaacggagtggg | 304 | CCUGCAGUCAAAACGGAGUGGG | 876 | CCCACUCCGUUUUGACUGCAGG |
| DS20-019B | 19 | ctgcagtcaaaacggagtgggg | 305 | CUGCAGUCAAAACGGAGUGGGG | 877 | CCCCACUCCGUUUUGACUGCAG |
| DS20-020B | 20 | ggtgcccagctcagggccagaa | 306 | GGUGCCCAGCUCAGGGCCAGAA | 878 | UUCUGGCCCUGAGCUGGGCACC |
| DS20-021B | 21 | gtgcccagctcagggccagaat | 307 | GUGCCCAGCUCAGGGCCAGAAU | 879 | AUUCUGGCCCUGAGCUGGGCAC |
| DS20-022B | 22 | tgcccagctcagggccagaatg | 308 | UGCCCAGCUCAGGGCCAGAAUG | 880 | CAUUCUGGCCCUGAGCUGGGCA |
| DS20-023B | 23 | gcccagctcagggccagaatga | 309 | GCCCAGCUCAGGGCCAGAAUGA | 881 | UCAUUCUGGCCCUGAGCUGGGC |
| DS20-024B | 24 | cccagctcagggccagaatgat | 310 | CCCAGCUCAGGGCCAGAAUGAU | 882 | AUCAUUCUGGCCCUGAGCUGGG |
| DS20-025B | 25 | ccagctcagggccagaatgatc | 311 | CCAGCUCAGGGCCAGAAUGAUC | 883 | GAUCAUUCUGGCCCUGAGCUGG |
| DS20-026B | 26 | cagctcagggccagaatgatcc | 312 | CAGCUCAGGGCCAGAAUGAUCC | 884 | GGAUCAUUCUGGCCCUGAGCUG |
| DS20-027B | 27 | agctcagggccagaatgatcct | 313 | AGCUCAGGGCCAGAAUGAUCCU | 885 | AGGAUCAUUCUGGCCCUGAGCU |
| DS20-028B | 28 | gctcagggccagaatgatccta | 314 | GCUCAGGGCCAGAAUGAUCCUA | 886 | UAGGAUCAUUCUGGCCCUGAGC |
| DS20-029B | 29 | ctcagggccagaatgatcctat | 315 | CUCAGGGCCAGAAUGAUCCUAU | 887 | AUAGGAUCAUUCUGGCCCUGAG |
| DS20-030B | 30 | tcagggccagaatgatcctatt | 316 | UCAGGGCCAGAAUGAUCCUAUU | 888 | AAUAGGAUCAUUCUGGCCCUGA |
| DS20-031B | 31 | cagggccagaatgatcctattc | 317 | CAGGGCCAGAAUGAUCCUAUUC | 889 | GAAUAGGAUCAUUCUGGCCCUG |
| DS20-032B | 32 | agggccagaatgatcctattcc | 318 | AGGGCCAGAAUGAUCCUAUUCC | 890 | GGAAUAGGAUCAUUCUGGCCCU |
| DS20-033B | 33 | gggccagaatgatcctattccc | 319 | GGGCCAGAAUGAUCCUAUUCCC | 891 | GGGAAUAGGAUCAUUCUGGCCC |
| DS20-034B | 34 | ggccagaatgatcctattcccg | 320 | GGCCAGAAUGAUCCUAUUCCCG | 892 | CGGGAAUAGGAUCAUUCUGGCC |
| DS20-035B | 35 | gccagaatgatcctattcccgg | 321 | GCCAGAAUGAUCCUAUUCCCGG | 893 | CCGGGAAUAGGAUCAUUCUGGC |
| DS20-036B | 36 | ccagaatgatcctattcccggc | 322 | CCAGAAUGAUCCUAUUCCCGGC | 894 | GCCGGGAAUAGGAUCAUUCUGG |
| DS20-037B | 37 | cagaatgatcctattcccggca | 323 | CAGAAUGAUCCUAUUCCCGGCA | 895 | UGCCGGGAAUAGGAUCAUUCUG |
| DS20-038B | 38 | agaatgatcctattcccggcac | 324 | AGAAUGAUCCUAUUCCCGGCAC | 896 | GUGCCGGGAAUAGGAUCAUUCU |
| DS20-039B | 39 | gaatgatcctattcccggcact | 325 | GAAUGAUCCUAUUCCCGGCACU | 897 | AGUGCCGGGAAUAGGAUCAUUC |
| DS20-040B | 40 | aatgatcctattcccggcactt | 326 | AAUGAUCCUAUUCCCGGCACUU | 898 | AAGUGCCGGGAAUAGGAUCAUU |
| DS20-041B | 41 | atgatcctattcccggcacttc | 327 | AUGAUCCUAUUCCCGGCACUUC | 899 | GAAGUGCCGGGAAUAGGAUCAU |
| DS20-042B | 42 | tgatcctattcccggcacttct | 328 | UGAUCCUAUUCCCGGCACUUCU | 900 | AGAAGUGCCGGGAAUAGGAUCA |
| DS20-043B | 43 | gatcctattcccggcacttctc | 329 | GAUCCUAUUCCCGGCACUUCUC | 901 | GAGAAGUGCCGGGAAUAGGAUC |
| DS20-044B | 44 | atcctattcccggcacttctca | 330 | AUCCUAUUCCCGGCACUUCUCA | 902 | UGAGAAGUGCCGGGAAUAGGAU |
| DS20-045B | 45 | tcctattcccggcacttctcag | 331 | UCCUAUUCCCGGCACUUCUCAG | 903 | CUGAGAAGUGCCGGGAAUAGGA |
| DS20-046B | 46 | cctattcccggcacttctcagt | 332 | CCUAUUCCCGGCACUUCUCAGU | 904 | ACUGAGAAGUGCCGGGAAUAGG |
| DS20-047B | 47 | ctattcccggcacttctcagtg | 333 | CUAUUCCCGGCACUUCUCAGUG | 905 | CACUGAGAAGUGCCGGGAAUAG |
| DS20-048B | 48 | tattcccggcacttctcagtga | 334 | UAUUCCCGGCACUUCUCAGUGA | 906 | UCACUGAGAAGUGCCGGGAAUA |
| DS20-049B | 49 | attcccggcacttctcagtgag | 335 | AUUCCCGGCACUUCUCAGUGAG | 907 | CUCACUGAGAAGUGCCGGGAAU |
| DS20-050B | 50 | ttcccggcacttctcagtgagg | 336 | UUCCCGGCACUUCUCAGUGAGG | 908 | CCUCACUGAGAAGUGCCGGGAA |
| DS20-051B | 51 | tcccggcacttctcagtgaggc | 337 | UCCCGGCACUUCUCAGUGAGGC | 909 | GCCUCACUGAGAAGUGCCGGGA |
| DS20-052B | 52 | cccggcacttctcagtgaggct | 338 | CCCGGCACUUCUCAGUGAGGCU | 910 | AGCCUCACUGAGAAGUGCCGGG |
| DS20-053B | 53 | ccggcacttctcagtgaggctc | 339 | CCGGCACUUCUCAGUGAGGCUC | 911 | GAGCCUCACUGAGAAGUGCCGG |
| DS20-054B | 54 | cggcacttctcagtgaggctct | 340 | CGGCACUUCUCAGUGAGGCUCU | 912 | AGAGCCUCACUGAGAAGUGCCG |
| DS20-055B | 55 | ggcacttctcagtgaggctctg | 341 | GGCACUUCUCAGUGAGGCUCUG | 913 | CAGAGCCUCACUGAGAAGUGCC |
| DS20-056B | 56 | gcacttctcagtgaggctctgt | 342 | GCACUUCUCAGUGAGGCUCUGU | 914 | ACAGAGCCUCACUGAGAAGUGC |
| DS20-057B | 57 | cacttctcagtgaggctctgtg | 343 | CACUUCUCAGUGAGGCUCUGUG | 915 | CACAGAGCCUCACUGAGAAGUG |
| DS20-058B | 58 | acttctcagtgaggctctgtgg | 344 | ACUUCUCAGUGAGGCUCUGUGG | 916 | CCACAGAGCCUCACUGAGAAGU |
| DS20-059B | 59 | cttctcagtgaggctctgtggc | 345 | CUUCUCAGUGAGGCUCUGUGGC | 917 | GCCACAGAGCCUCACUGAGAAG |
| DS20-060B | 60 | ttctcagtgaggctctgtggct | 346 | UUCUCAGUGAGGCUCUGUGGCU | 918 | AGCCACAGAGCCUCACUGAGAA |
| DS20-061B | 61 | tctcagtgaggctctgtggctc | 347 | UCUCAGUGAGGCUCUGUGGCUC | 919 | GAGCCACAGAGCCUCACUGAGA |
| DS20-062B | 62 | ctcagtgaggctctgtggctca | 348 | CUCAGUGAGGCUCUGUGGCUCA | 920 | UGAGCCACAGAGCCUCACUGAG |
| DS20-063B | 63 | tcagtgaggctctgtggctcac | 349 | UCAGUGAGGCUCUGUGGCUCAC | 921 | GUGAGCCACAGAGCCUCACUGA |
| DS20-064B | 64 | cagtgaggctctgtggctcacc | 350 | CAGUGAGGCUCUGUGGCUCACC | 922 | GGUGAGCCACAGAGCCUCACUG |
| DS20-065B | 65 | agtgaggctctgtggctcacct | 351 | AGUGAGGCUCUGUGGCUCACCU | 923 | AGGUGAGCCACAGAGCCUCACU |
| DS20-066B | 66 | gtgaggctctgtggctcaccta | 352 | GUGAGGCUCUGUGGCUCACCUA | 924 | UAGGUGAGCCACAGAGCCUCAC |
| DS20-067B | 67 | tgaggctctgtggctcacctaa | 353 | UGAGGCUCUGUGGCUCACCUAA | 925 | UUAGGUGAGCCACAGAGCCUCA |
| DS20-068B | 68 | gaggctctgtggctcacctaag | 354 | GAGGCUCUGUGGCUCACCUAAG | 926 | CUUAGGUGAGCCACAGAGCCUC |
| DS20-069B | 69 | aggctctgtggctcacctaaga | 355 | AGGCUCUGUGGCUCACCUAAGA | 927 | UCUUAGGUGAGCCACAGAGCCU |
| DS20-070B | 70 | ggctctgtggctcacctaagaa | 356 | GGCUCUGUGGCUCACCUAAGAA | 928 | UUCUUAGGUGAGCCACAGAGCC |
| DS20-071B | 71 | gctctgtggctcacctaagaaa | 357 | GCUCUGUGGCUCACCUAAGAAA | 929 | UUUCUUAGGUGAGCCACAGAGC |
| DS20-072B | 72 | ctctgtggctcacctaagaaac | 358 | CUCUGUGGCUCACCUAAGAAAC | 930 | GUUUCUUAGGUGAGCCACAGAG |
| DS20-073B | 73 | tctgtggctcacctaagaaacc | 359 | UCUGUGGCUCACCUAAGAAACC | 931 | GGUUUCUUAGGUGAGCCACAGA |
| DS20-074B | 74 | ctgtggctcacctaagaaacca | 360 | CUGUGGCUCACCUAAGAAACCA | 932 | UGGUUUCUUAGGUGAGCCACAG |
| DS20-075B | 75 | tgtggctcacctaagaaaccag | 361 | UGUGGCUCACCUAAGAAACCAG | 933 | CUGGUUUCUUAGGUGAGCCACA |
| DS20-076B | 76 | gtggctcacctaagaaaccagc | 362 | GUGGCUCACCUAAGAAACCAGC | 934 | GCUGGUUUCUUAGGUGAGCCAC |
| DS20-077B | 77 | tggctcacctaagaaaccagcc | 363 | UGGCUCACCUAAGAAACCAGCC | 935 | GGCUGGUUUCUUAGGUGAGCCA |
| DS20-078B | 78 | ggctcacctaagaaaccagcct | 364 | GGCUCACCUAAGAAACCAGCCU | 936 | AGGCUGGUUUCUUAGGUGAGCC |
| DS20-079B | 79 | gctcacctaagaaaccagcctc | 365 | GCUCACCUAAGAAACCAGCCUC | 937 | GAGGCUGGUUUCUUAGGUGAGC |
| DS20-080B | 80 | ctcacctaagaaaccagcctcc | 366 | CUCACCUAAGAAACCAGCCUCC | 938 | GGAGGCUGGUUUCUUAGGUGAG |
| DS20-081B | 81 | tcacctaagaaaccagcctccc | 367 | UCACCUAAGAAACCAGCCUCCC | 939 | GGGAGGCUGGUUUCUUAGGUGA |
| DS20-082B | 82 | cacctaagaaaccagcctccct | 368 | CACCUAAGAAACCAGCCUCCCU | 940 | AGGGAGGCUGGUUUCUUAGGUG |
| DS20-083B | 83 | acctaagaaaccagcctccctt | 369 | ACCUAAGAAACCAGCCUCCCUU | 941 | AAGGGAGGCUGGUUUCUUAGGU |
| DS20-084B | 84 | cctaagaaaccagcctcccttg | 370 | CCUAAGAAACCAGCCUCCCUUG | 942 | CAAGGGAGGCUGGUUUCUUAGG |
| DS20-085B | 85 | ctaagaaaccagcctcccttgc | 371 | CUAAGAAACCAGCCUCCCUUGC | 943 | GCAAGGGAGGCUGGUUUCUUAG |
| DS20-086B | 86 | taagaaaccagcctcccttgca | 372 | UAAGAAACCAGCCUCCCUUGCA | 944 | UGCAAGGGAGGCUGGUUUCUUA |
| DS20-087B | 87 | aagaaaccagcctcccttgcag | 373 | AAGAAACCAGCCUCCCUUGCAG | 945 | CUGCAAGGGAGGCUGGUUUCUU |
| DS20-088B | 88 | agaaaccagcctcccttgcagg | 374 | AGAAACCAGCCUCCCUUGCAGG | 946 | CCUGCAAGGGAGGCUGGUUUCU |
| DS20-089B | 89 | gaaaccagcctcccttgcaggc | 375 | GAAACCAGCCUCCCUUGCAGGC | 947 | GCCUGCAAGGGAGGCUGGUUUC |
| DS20-090B | 90 | aaaccagcctcccttgcaggca | 376 | AAACCAGCCUCCCUUGCAGGCA | 948 | UGCCUGCAAGGGAGGCUGGUUU |
| DS20-091B | 91 | aaccagcctcccttgcaggcaa | 377 | AACCAGCCUCCCUUGCAGGCAA | 949 | UUGCCUGCAAGGGAGGCUGGUU |
| DS20-092B | 92 | accagcctcccttgcaggcaac | 378 | ACCAGCCUCCCUUGCAGGCAAC | 950 | GUUGCCUGCAAGGGAGGCUGGU |
| DS20-093B | 93 | ccagcctcccttgcaggcaacg | 379 | CCAGCCUCCCUUGCAGGCAACG | 951 | CGUUGCCUGCAAGGGAGGCUGG |
| DS20-094B | 94 | cagcctcccttgcaggcaacgg | 380 | CAGCCUCCCUUGCAGGCAACGG | 952 | CCGUUGCCUGCAAGGGAGGCUG |
| DS20-095B | 95 | agcctcccttgcaggcaacggc | 381 | AGCCUCCCUUGCAGGCAACGGC | 953 | GCCGUUGCCUGCAAGGGAGGCU |
| DS20-096B | 96 | cctcccttgcaggcaacggcct | 382 | CCUCCCUUGCAGGCAACGGCCU | 954 | AGGCCGUUGCCUGCAAGGGAGG |
| DS20-097B | 97 | ctcccttgcaggcaacggccta | 383 | CUCCCUUGCAGGCAACGGCCUA | 955 | UAGGCCGUUGCCUGCAAGGGAG |
| DS20-098B | 98 | tcccttgcaggcaacggcctag | 384 | UCCCUUGCAGGCAACGGCCUAG | 956 | CUAGGCCGUUGCCUGCAAGGGA |
| DS20-099B | 99 | cccttgcaggcaacggcctagc | 385 | CCCUUGCAGGCAACGGCCUAGC | 957 | GCUAGGCCGUUGCCUGCAAGGG |
| DS20-100B | 100 | ccttgcaggcaacggcctagct | 386 | CCUUGCAGGCAACGGCCUAGCU | 958 | AGCUAGGCCGUUGCCUGCAAGG |
| DS20-101B | 101 | cttgcaggcaacggcctagctg | 387 | CUUGCAGGCAACGGCCUAGCUG | 959 | CAGCUAGGCCGUUGCCUGCAAG |
| DS20-102B | 102 | ttgcaggcaacggcctagctgg | 388 | UUGCAGGCAACGGCCUAGCUGG | 960 | CCAGCUAGGCCGUUGCCUGCAA |
| DS20-103B | 103 | tgcaggcaacggcctagctggc | 389 | UGCAGGCAACGGCCUAGCUGGC | 961 | GCCAGCUAGGCCGUUGCCUGCA |
| DS20-104B | 104 | caggcaacggcctagctggcct | 390 | CAGGCAACGGCCUAGCUGGCCU | 962 | AGGCCAGCUAGGCCGUUGCCUG |
| DS20-105B | 105 | aggcaacggcctagctggcctg | 391 | AGGCAACGGCCUAGCUGGCCUG | 963 | CAGGCCAGCUAGGCCGUUGCCU |
| DS20-106B | 106 | gcaacggcctagctggcctggt | 392 | GCAACGGCCUAGCUGGCCUGGU | 964 | ACCAGGCCAGCUAGGCCGUUGC |
| DS20-107B | 107 | caacggcctagctggcctggtc | 393 | CAACGGCCUAGCUGGCCUGGUC | 965 | GACCAGGCCAGCUAGGCCGUUG |
| DS20-108B | 108 | aacggcctagctggcctggtct | 394 | AACGGCCUAGCUGGCCUGGUCU | 966 | AGACCAGGCCAGCUAGGCCGUU |
| DS20-109B | 109 | acggcctagctggcctggtctg | 395 | ACGGCCUAGCUGGCCUGGUCUG | 967 | CAGACCAGGCCAGCUAGGCCGU |
| DS20-110B | 110 | ggcctagctggcctggtctgga | 396 | GGCCUAGCUGGCCUGGUCUGGA | 968 | UCCAGACCAGGCCAGCUAGGCC |
| DS20-111B | 111 | gcctagctggcctggtctggag | 397 | GCCUAGCUGGCCUGGUCUGGAG | 969 | CUCCAGACCAGGCCAGCUAGGC |
| DS20-112B | 112 | cctagctggcctggtctggagg | 398 | CCUAGCUGGCCUGGUCUGGAGG | 970 | CCUCCAGACCAGGCCAGCUAGG |
| DS20-113B | 113 | ctagctggcctggtctggaggc | 399 | CUAGCUGGCCUGGUCUGGAGGC | 971 | GCCUCCAGACCAGGCCAGCUAG |
| DS20-114B | 114 | tagctggcctggtctggaggct | 400 | UAGCUGGCCUGGUCUGGAGGCU | 972 | AGCCUCCAGACCAGGCCAGCUA |
| DS20-115B | 115 | agctggcctggtctggaggctc | 401 | AGCUGGCCUGGUCUGGAGGCUC | 973 | GAGCCUCCAGACCAGGCCAGCU |
| DS20-116B | 116 | gctggcctggtctggaggctct | 402 | GCUGGCCUGGUCUGGAGGCUCU | 974 | AGAGCCUCCAGACCAGGCCAGC |
| DS20-117B | 117 | ctggcctggtctggaggctctc | 403 | CUGGCCUGGUCUGGAGGCUCUC | 975 | GAGAGCCUCCAGACCAGGCCAG |
| DS20-118B | 118 | tttacatccacacccaagatac | 404 | UUUACAUCCACACCCAAGAUAC | 976 | GUAUCUUGGGUGUGGAUGUAAA |
| DS20-119B | 119 | tcttgagatttgactcgcatga | 405 | UCUUGAGAUUUGACUCGCAUGA | 977 | UCAUGCGAGUCAAAUCUCAAGA |
| DS20-120B | 120 | cttgagatttgactcgcatgat | 406 | CUUGAGAUUUGACUCGCAUGAU | 978 | AUCAUGCGAGUCAAAUCUCAAG |
| DS20-121B | 121 | tgagatttgactcgcatgattg | 407 | UGAGAUUUGACUCGCAUGAUUG | 979 | CAAUCAUGCGAGUCAAAUCUCA |
| DS20-122B | 122 | gagatttgactcgcatgattgc | 408 | GAGAUUUGACUCGCAUGAUUGC | 980 | GCAAUCAUGCGAGUCAAAUCUC |
| DS20-123B | 123 | agatttgactcgcatgattgct | 409 | AGAUUUGACUCGCAUGAUUGCU | 981 | AGCAAUCAUGCGAGUCAAAUCU |
| DS20-124B | 124 | gatttgactcgcatgattgcta | 410 | GAUUUGACUCGCAUGAUUGCUA | 982 | UAGCAAUCAUGCGAGUCAAAUC |
| DS20-125B | 125 | tttgactcgcatgattgctatg | 411 | UUUGACUCGCAUGAUUGCUAUG | 983 | CAUAGCAAUCAUGCGAGUCAAA |
| DS20-126B | 126 | ttgactcgcatgattgctatgg | 412 | UUGACUCGCAUGAUUGCUAUGG | 984 | CCAUAGCAAUCAUGCGAGUCAA |
| DS20-127B | 127 | tgactcgcatgattgctatggg | 413 | UGACUCGCAUGAUUGCUAUGGG | 985 | CCCAUAGCAAUCAUGCGAGUCA |
| DS20-128B | 128 | gactcgcatgattgctatggga | 414 | GACUCGCAUGAUUGCUAUGGGA | 986 | UCCCAUAGCAAUCAUGCGAGUC |
| DS20-129B | 129 | actcgcatgattgctatgggac | 415 | ACUCGCAUGAUUGCUAUGGGAC | 987 | GUCCCAUAGCAAUCAUGCGAGU |
| DS20-130B | 130 | ctcgcatgattgctatgggaca | 416 | CUCGCAUGAUUGCUAUGGGACA | 988 | UGUCCCAUAGCAAUCAUGCGAG |
| DS20-131B | 131 | tcgcatgattgctatgggacaa | 417 | UCGCAUGAUUGCUAUGGGACAA | 989 | UUGUCCCAUAGCAAUCAUGCGA |
| DS20-132B | 132 | cgcatgattgctatgggacaag | 418 | CGCAUGAUUGCUAUGGGACAAG | 990 | CUUGUCCCAUAGCAAUCAUGCG |
| DS20-133B | 133 | gcatgattgctatgggacaagt | 419 | GCAUGAUUGCUAUGGGACAAGU | 991 | ACUUGUCCCAUAGCAAUCAUGC |
| DS20-134B | 134 | catgattgctatgggacaagtt | 420 | CAUGAUUGCUAUGGGACAAGUU | 992 | AACUUGUCCCAUAGCAAUCAUG |
| DS20-135B | 135 | gattgctatgggacaagttttc | 421 | GAUUGCUAUGGGACAAGUUUUC | 993 | GAAAACUUGUCCCAUAGCAAUC |
| DS20-136B | 136 | tgctatgggacaagttttcatc | 422 | UGCUAUGGGACAAGUUUUCAUC | 994 | GAUGAAAACUUGUCCCAUAGCA |
| DS20-137B | 137 | gctatgggacaagttttcatct | 423 | GCUAUGGGACAAGUUUUCAUCU | 995 | AGAUGAAAACUUGUCCCAUAGC |
| DS20-138B | 138 | ctatgggacaagttttcatctg | 424 | CUAUGGGACAAGUUUUCAUCUG | 996 | CAGAUGAAAACUUGUCCCAUAG |
| DS20-139B | 139 | tatgggacaagttttcatctgc | 425 | UAUGGGACAAGUUUUCAUCUGC | 997 | GCAGAUGAAAACUUGUCCCAUA |
| DS20-140B | 140 | atgggacaagttttcatctgca | 426 | AUGGGACAAGUUUUCAUCUGCA | 998 | UGCAGAUGAAAACUUGUCCCAU |
| DS20-141B | 141 | tgggacaagttttcatctgcag | 427 | UGGGACAAGUUUUCAUCUGCAG | 999 | CUGCAGAUGAAAACUUGUCCCA |
| DS20-142B | 142 | gggacaagttttcatctgcagt | 428 | GGGACAAGUUUUCAUCUGCAGU | 1000 | ACUGCAGAUGAAAACUUGUCCC |
| DS20-143B | 143 | ggacaagttttcatctgcagtt | 429 | GGACAAGUUUUCAUCUGCAGUU | 1001 | AACUGCAGAUGAAAACUUGUCC |
| DS20-144B | 144 | ctgcagtttaaatctgtttccc | 430 | CUGCAGUUUAAAUCUGUUUCCC | 1002 | GGGAAACAGAUUUAAACUGCAG |
| DS20-145B | 145 | taggggtttggaattctagatc | 431 | UAGGGGUUUGGAAUUCUAGAUC | 1003 | GAUCUAGAAUUCCAAACCCCUA |
| DS20-146B | 146 | aggggtttggaattctagatcg | 432 | AGGGGUUUGGAAUUCUAGAUCG | 1004 | CGAUCUAGAAUUCCAAACCCCU |
| DS20-147B | 147 | ggggtttggaattctagatcgt | 433 | GGGGUUUGGAAUUCUAGAUCGU | 1005 | ACGAUCUAGAAUUCCAAACCCC |
| DS20-148B | 148 | gggtttggaattctagatcgta | 434 | GGGUUUGGAAUUCUAGAUCGUA | 1006 | UACGAUCUAGAAUUCCAAACCC |
| DS20-149B | 149 | gatcgtatttgaagtgttggtg | 435 | GAUCGUAUUUGAAGUGUUGGUG | 1007 | CACCAACACUUCAAAUACGAUC |
| DS20-150B | 150 | atcgtatttgaagtgttggtgc | 436 | AUCGUAUUUGAAGUGUUGGUGC | 1008 | GCACCAACACUUCAAAUACGAU |
| DS20-151B | 151 | tcgtatttgaagtgttggtgcc | 437 | UCGUAUUUGAAGUGUUGGUGCC | 1009 | GGCACCAACACUUCAAAUACGA |
| DS20-152B | 152 | cgtatttgaagtgttggtgcca | 438 | CGUAUUUGAAGUGUUGGUGCCA | 1010 | UGGCACCAACACUUCAAAUACG |
| DS20-153B | 153 | gtatttgaagtgttggtgccac | 439 | GUAUUUGAAGUGUUGGUGCCAC | 1011 | GUGGCACCAACACUUCAAAUAC |
| DS20-154B | 154 | tatttgaagtgttggtgccaca | 440 | UAUUUGAAGUGUUGGUGCCACA | 1012 | UGUGGCACCAACACUUCAAAUA |
| DS20-155B | 155 | atttgaagtgttggtgccacac | 441 | AUUUGAAGUGUUGGUGCCACAC | 1013 | GUGUGGCACCAACACUUCAAAU |
| DS20-156B | 156 | cacaccttaacacctgcacgct | 442 | CACACCUUAACACCUGCACGCU | 1014 | AGCGUGCAGGUGUUAAGGUGUG |
| DS20-157B | 157 | acaccttaacacctgcacgctg | 443 | ACACCUUAACACCUGCACGCUG | 1015 | CAGCGUGCAGGUGUUAAGGUGU |
| DS20-158B | 158 | caccttaacacctgcacgctgg | 444 | CACCUUAACACCUGCACGCUGG | 1016 | CCAGCGUGCAGGUGUUAAGGUG |
| DS20-159B | 159 | accttaacacctgcacgctggc | 445 | ACCUUAACACCUGCACGCUGGC | 1017 | GCCAGCGUGCAGGUGUUAAGGU |
| DS20-160B | 160 | ccttaacacctgcacgctggca | 446 | CCUUAACACCUGCACGCUGGCA | 1018 | UGCCAGCGUGCAGGUGUUAAGG |
| DS20-161B | 161 | cttaacacctgcacgctggcaa | 447 | CUUAACACCUGCACGCUGGCAA | 1019 | UUGCCAGCGUGCAGGUGUUAAG |
| DS20-162B | 162 | ttaacacctgcacgctggcaac | 448 | UUAACACCUGCACGCUGGCAAC | 1020 | GUUGCCAGCGUGCAGGUGUUAA |
| DS20-163B | 163 | taacacctgcacgctggcaaca | 449 | UAACACCUGCACGCUGGCAACA | 1021 | UGUUGCCAGCGUGCAGGUGUUA |
| DS20-164B | 164 | aacacctgcacgctggcaacaa | 450 | AACACCUGCACGCUGGCAACAA | 1022 | UUGUUGCCAGCGUGCAGGUGUU |
| DS20-165B | 165 | acacctgcacgctggcaacaaa | 451 | ACACCUGCACGCUGGCAACAAA | 1023 | UUUGUUGCCAGCGUGCAGGUGU |
| DS20-166B | 166 | cacctgcacgctggcaacaaaa | 452 | CACCUGCACGCUGGCAACAAAA | 1024 | UUUUGUUGCCAGCGUGCAGGUG |
| DS20-167B | 167 | acctgcacgctggcaacaaaac | 453 | ACCUGCACGCUGGCAACAAAAC | 1025 | GUUUUGUUGCCAGCGUGCAGGU |
| DS20-168B | 168 | cctgcacgctggcaacaaaacc | 454 | CCUGCACGCUGGCAACAAAACC | 1026 | GGUUUUGUUGCCAGCGUGCAGG |
| DS20-169B | 169 | ctgcacgctggcaacaaaaccg | 455 | CUGCACGCUGGCAACAAAACCG | 1027 | CGGUUUUGUUGCCAGCGUGCAG |
| DS20-170B | 170 | tgcacgctggcaacaaaaccgt | 456 | UGCACGCUGGCAACAAAACCGU | 1028 | ACGGUUUUGUUGCCAGCGUGCA |
| DS20-171B | 171 | gcacgctggcaacaaaaccgtc | 457 | GCACGCUGGCAACAAAACCGUC | 1029 | GACGGUUUUGUUGCCAGCGUGC |
| DS20-172B | 172 | cacgctggcaacaaaaccgtcc | 458 | CACGCUGGCAACAAAACCGUCC | 1030 | GGACGGUUUUGUUGCCAGCGUG |
| DS20-173B | 173 | acgctggcaacaaaaccgtccg | 459 | ACGCUGGCAACAAAACCGUCCG | 1031 | CGGACGGUUUUGUUGCCAGCGU |
| DS20-174B | 174 | cgctggcaacaaaaccgtccgc | 460 | CGCUGGCAACAAAACCGUCCGC | 1032 | GCGGACGGUUUUGUUGCCAGCG |
| DS20-175B | 175 | gctggcaacaaaaccgtccgct | 461 | GCUGGCAACAAAACCGUCCGCU | 1033 | AGCGGACGGUUUUGUUGCCAGC |
| DS20-176B | 176 | ctggcaacaaaaccgtccgctc | 462 | CUGGCAACAAAACCGUCCGCUC | 1034 | GAGCGGACGGUUUUGUUGCCAG |
| DS20-177B | 177 | tggcaacaaaaccgtccgctct | 463 | UGGCAACAAAACCGUCCGCUCU | 1035 | AGAGCGGACGGUUUUGUUGCCA |
| DS20-178B | 178 | ggcaacaaaaccgtccgctctg | 464 | GGCAACAAAACCGUCCGCUCUG | 1036 | CAGAGCGGACGGUUUUGUUGCC |
| DS20-179B | 179 | gcaacaaaaccgtccgctctgc | 465 | GCAACAAAACCGUCCGCUCUGC | 1037 | GCAGAGCGGACGGUUUUGUUGC |
| DS20-180B | 180 | caacaaaaccgtccgctctgca | 466 | CAACAAAACCGUCCGCUCUGCA | 1038 | UGCAGAGCGGACGGUUUUGUUG |
| DS20-181B | 181 | aacaaaaccgtccgctctgcag | 467 | AACAAAACCGUCCGCUCUGCAG | 1039 | CUGCAGAGCGGACGGUUUUGUU |
| DS20-182B | 182 | acaaaaccgtccgctctgcagc | 468 | ACAAAACCGUCCGCUCUGCAGC | 1040 | GCUGCAGAGCGGACGGUUUUGU |
| DS20-183B | 183 | caaaaccgtccgctctgcagca | 469 | CAAAACCGUCCGCUCUGCAGCA | 1041 | UGCUGCAGAGCGGACGGUUUUG |
| DS20-184B | 184 | aaaaccgtccgctctgcagcac | 470 | AAAACCGUCCGCUCUGCAGCAC | 1042 | GUGCUGCAGAGCGGACGGUUUU |
| DS20-185B | 185 | aaaccgtccgctctgcagcaca | 471 | AAACCGUCCGCUCUGCAGCACA | 1043 | UGUGCUGCAGAGCGGACGGUUU |
| DS20-186B | 186 | aaccgtccgctctgcagcacag | 472 | AACCGUCCGCUCUGCAGCACAG | 1044 | CUGUGCUGCAGAGCGGACGGUU |
| DS20-187B | 187 | accgtccgctctgcagcacagc | 473 | ACCGUCCGCUCUGCAGCACAGC | 1045 | GCUGUGCUGCAGAGCGGACGGU |
| DS20-188B | 188 | ccgtccgctctgcagcacagct | 474 | CCGUCCGCUCUGCAGCACAGCU | 1046 | AGCUGUGCUGCAGAGCGGACGG |
| DS20-189B | 189 | cgtccgctctgcagcacagctg | 475 | CGUCCGCUCUGCAGCACAGCUG | 1047 | CAGCUGUGCUGCAGAGCGGACG |
| DS20-190B | 190 | gtccgctctgcagcacagctgg | 476 | GUCCGCUCUGCAGCACAGCUGG | 1048 | CCAGCUGUGCUGCAGAGCGGAC |
| DS20-191B | 191 | tccgctctgcagcacagctggg | 477 | UCCGCUCUGCAGCACAGCUGGG | 1049 | CCCAGCUGUGCUGCAGAGCGGA |
| DS20-192B | 192 | cgctctgcagcacagctggggt | 478 | CGCUCUGCAGCACAGCUGGGGU | 1050 | ACCCCAGCUGUGCUGCAGAGCG |
| DS20-193B | 193 | gctctgcagcacagctggggtc | 479 | GCUCUGCAGCACAGCUGGGGUC | 1051 | GACCCCAGCUGUGCUGCAGAGC |
| DS20-194B | 194 | ctctgcagcacagctggggtca | 480 | CUCUGCAGCACAGCUGGGGUCA | 1052 | UGACCCCAGCUGUGCUGCAGAG |
| DS20-195B | 195 | tctgcagcacagctggggtcac | 481 | UCUGCAGCACAGCUGGGGUCAC | 1053 | GUGACCCCAGCUGUGCUGCAGA |
| DS20-196B | 196 | ctgcagcacagctggggtcacc | 482 | CUGCAGCACAGCUGGGGUCACC | 1054 | GGUGACCCCAGCUGUGCUGCAG |
| DS20-197B | 197 | tgcagcacagctggggtcacct | 483 | UGCAGCACAGCUGGGGUCACCU | 1055 | AGGUGACCCCAGCUGUGCUGCA |
| DS20-198B | 198 | gcagcacagctggggtcacctg | 484 | GCAGCACAGCUGGGGUCACCUG | 1056 | CAGGUGACCCCAGCUGUGCUGC |
| DS20-199B | 199 | cagcacagctggggtcacctga | 485 | CAGCACAGCUGGGGUCACCUGA | 1057 | UCAGGUGACCCCAGCUGUGCUG |
| DS20-200B | 200 | agcacagctggggtcacctgac | 486 | AGCACAGCUGGGGUCACCUGAC | 1058 | GUCAGGUGACCCCAGCUGUGCU |
| DS20-201B | 201 | gcacagctggggtcacctgacc | 487 | GCACAGCUGGGGUCACCUGACC | 1059 | GGUCAGGUGACCCCAGCUGUGC |
| DS20-202B | 202 | cacagctggggtcacctgacct | 488 | CACAGCUGGGGUCACCUGACCU | 1060 | AGGUCAGGUGACCCCAGCUGUG |
| DS20-203B | 203 | acagctggggtcacctgacctt | 489 | ACAGCUGGGGUCACCUGACCUU | 1061 | AAGGUCAGGUGACCCCAGCUGU |
| DS20-204B | 204 | cagctggggtcacctgaccttt | 490 | CAGCUGGGGUCACCUGACCUUU | 1062 | AAAGGUCAGGUGACCCCAGCUG |
| DS20-205B | 205 | agctggggtcacctgacctttc | 491 | AGCUGGGGUCACCUGACCUUUC | 1063 | GAAAGGUCAGGUGACCCCAGCU |
| DS20-206B | 206 | gctggggtcacctgacctttct | 492 | GCUGGGGUCACCUGACCUUUCU | 1064 | AGAAAGGUCAGGUGACCCCAGC |
| DS20-207B | 207 | ctggggtcacctgacctttctc | 493 | CUGGGGUCACCUGACCUUUCUC | 1065 | GAGAAAGGUCAGGUGACCCCAG |
| DS20-208B | 208 | tggggtcacctgacctttctcc | 494 | UGGGGUCACCUGACCUUUCUCC | 1066 | GGAGAAAGGUCAGGUGACCCCA |
| DS20-209B | 209 | ggggtcacctgacctttctcct | 495 | GGGGUCACCUGACCUUUCUCCU | 1067 | AGGAGAAAGGUCAGGUGACCCC |
| DS20-210B | 210 | gggtcacctgacctttctcctg | 496 | GGGUCACCUGACCUUUCUCCUG | 1068 | CAGGAGAAAGGUCAGGUGACCC |
| DS20-211B | 211 | ggtcacctgacctttctcctgt | 497 | GGUCACCUGACCUUUCUCCUGU | 1069 | ACAGGAGAAAGGUCAGGUGACC |
| DS20-212B | 212 | gtcacctgacctttctcctgtc | 498 | GUCACCUGACCUUUCUCCUGUC | 1070 | GACAGGAGAAAGGUCAGGUGAC |
| DS20-213B | 213 | tcacctgacctttctcctgtcc | 499 | UCACCUGACCUUUCUCCUGUCC | 1071 | GGACAGGAGAAAGGUCAGGUGA |
| DS20-214B | 214 | cacctgacctttctcctgtccc | 500 | CACCUGACCUUUCUCCUGUCCC | 1072 | GGGACAGGAGAAAGGUCAGGUG |
| DS20-215B | 215 | ccccacttgagctcagtggctg | 501 | CCCCACUUGAGCUCAGUGGCUG | 1073 | CAGCCACUGAGCUCAAGUGGGG |
| DS20-216B | 216 | cccacttgagctcagtggctgg | 502 | CCCACUUGAGCUCAGUGGCUGG | 1074 | CCAGCCACUGAGCUCAAGUGGG |
| DS20-217B | 217 | ccacttgagctcagtggctggg | 503 | CCACUUGAGCUCAGUGGCUGGG | 1075 | CCCAGCCACUGAGCUCAAGUGG |
| DS20-218B | 218 | cacttgagctcagtggctgggc | 504 | CACUUGAGCUCAGUGGCUGGGC | 1076 | GCCCAGCCACUGAGCUCAAGUG |
| DS20-219B | 219 | acttgagctcagtggctgggca | 505 | ACUUGAGCUCAGUGGCUGGGCA | 1077 | UGCCCAGCCACUGAGCUCAAGU |
| DS20-220B | 220 | cttgagctcagtggctgggcag | 506 | CUUGAGCUCAGUGGCUGGGCAG | 1078 | CUGCCCAGCCACUGAGCUCAAG |
| DS20-221B | 221 | ttgagctcagtggctgggcagc | 507 | UUGAGCUCAGUGGCUGGGCAGC | 1079 | GCUGCCCAGCCACUGAGCUCAA |
| DS20-222B | 222 | tgagctcagtggctgggcagca | 508 | UGAGCUCAGUGGCUGGGCAGCA | 1080 | UGCUGCCCAGCCACUGAGCUCA |
| DS20-223B | 223 | gagctcagtggctgggcagcag | 509 | GAGCUCAGUGGCUGGGCAGCAG | 1081 | CUGCUGCCCAGCCACUGAGCUC |
| DS20-224B | 224 | agctcagtggctgggcagcagg | 510 | AGCUCAGUGGCUGGGCAGCAGG | 1082 | CCUGCUGCCCAGCCACUGAGCU |
| DS20-225B | 225 | tcagtggctgggcagcagggga | 511 | UCAGUGGCUGGGCAGCAGGGGA | 1083 | UCCCCUGCUGCCCAGCCACUGA |
| DS20-226B | 226 | cagtggctgggcagcaggggat | 512 | CAGUGGCUGGGCAGCAGGGGAU | 1084 | AUCCCCUGCUGCCCAGCCACUG |
| DS20-227B | 227 | agtggctgggcagcaggggatg | 513 | AGUGGCUGGGCAGCAGGGGAUG | 1085 | CAUCCCCUGCUGCCCAGCCACU |
| DS20-228B | 228 | tggctgggcagcaggggatgca | 514 | UGGCUGGGCAGCAGGGGAUGCA | 1086 | UGCAUCCCCUGCUGCCCAGCCA |
| DS20-229B | 229 | ggctgggcagcaggggatgcat | 515 | GGCUGGGCAGCAGGGGAUGCAU | 1087 | AUGCAUCCCCUGCUGCCCAGCC |
| DS20-230B | 230 | gctgggcagcaggggatgcatg | 516 | GCUGGGCAGCAGGGGAUGCAUG | 1088 | CAUGCAUCCCCUGCUGCCCAGC |
| DS20-231B | 231 | ctgggcagcaggggatgcatgg | 517 | CUGGGCAGCAGGGGAUGCAUGG | 1089 | CCAUGCAUCCCCUGCUGCCCAG |
| DS20-232B | 232 | tgggcagcaggggatgcatggc | 518 | UGGGCAGCAGGGGAUGCAUGGC | 1090 | GCCAUGCAUCCCCUGCUGCCCA |
| DS20-233B | 233 | ggcagcaggggatgcatggcca | 519 | GGCAGCAGGGGAUGCAUGGCCA | 1091 | UGGCCAUGCAUCCCCUGCUGCC |
| DS20-234B | 234 | gcagcaggggatgcatggccac | 520 | GCAGCAGGGGAUGCAUGGCCAC | 1092 | GUGGCCAUGCAUCCCCUGCUGC |
| DS20-235B | 235 | cagcaggggatgcatggccact | 521 | CAGCAGGGGAUGCAUGGCCACU | 1093 | AGUGGCCAUGCAUCCCCUGCUG |
| DS20-236B | 236 | agcaggggatgcatggccactg | 522 | AGCAGGGGAUGCAUGGCCACUG | 1094 | CAGUGGCCAUGCAUCCCCUGCU |
| DS20-237B | 237 | gcaggggatgcatggccactgg | 523 | GCAGGGGAUGCAUGGCCACUGG | 1095 | CCAGUGGCCAUGCAUCCCCUGC |
| DS20-238B | 238 | caggggatgcatggccactggc | 524 | CAGGGGAUGCAUGGCCACUGGC | 1096 | GCCAGUGGCCAUGCAUCCCCUG |
| DS20-239B | 239 | aggggatgcatggccactggcc | 525 | AGGGGAUGCAUGGCCACUGGCC | 1097 | GGCCAGUGGCCAUGCAUCCCCU |
| DS20-240B | 240 | atgcatggccactggccggcca | 526 | AUGCAUGGCCACUGGCCGGCCA | 1098 | UGGCCGGCCAGUGGCCAUGCAU |
| DS20-241B | 241 | cggccaggtgcagctctcagct | 527 | CGGCCAGGUGCAGCUCUCAGCU | 1099 | AGCUGAGAGCUGCACCUGGCCG |
| DS20-242B | 242 | ggccaggtgcagctctcagctg | 528 | GGCCAGGUGCAGCUCUCAGCUG | 1100 | CAGCUGAGAGCUGCACCUGGCC |
| DS20-243B | 243 | gccaggtgcagctctcagctgg | 529 | GCCAGGUGCAGCUCUCAGCUGG | 1101 | CCAGCUGAGAGCUGCACCUGGC |
| DS20-244B | 244 | ccaggtgcagctctcagctggg | 530 | CCAGGUGCAGCUCUCAGCUGGG | 1102 | CCCAGCUGAGAGCUGCACCUGG |
| DS20-245B | 245 | caggtgcagctctcagctgggg | 531 | CAGGUGCAGCUCUCAGCUGGGG | 1103 | CCCCAGCUGAGAGCUGCACCUG |
| DS20-246B | 246 | aggtgcagctctcagctggggt | 532 | AGGUGCAGCUCUCAGCUGGGGU | 1104 | ACCCCAGCUGAGAGCUGCACCU |
| DS20-247B | 247 | ggtgcagctctcagctggggtg | 533 | GGUGCAGCUCUCAGCUGGGGUG | 1105 | CACCCCAGCUGAGAGCUGCACC |
| DS20-248B | 248 | gtgcagctctcagctggggtgt | 534 | GUGCAGCUCUCAGCUGGGGUGU | 1106 | ACACCCCAGCUGAGAGCUGCAC |
| DS20-249B | 249 | tgcagctctcagctggggtgtt | 535 | UGCAGCUCUCAGCUGGGGUGUU | 1107 | AACACCCCAGCUGAGAGCUGCA |
| DS20-250B | 250 | gcagctctcagctggggtgttc | 536 | GCAGCUCUCAGCUGGGGUGUUC | 1108 | GAACACCCCAGCUGAGAGCUGC |
| DS20-251B | 251 | cagctctcagctggggtgttca | 537 | CAGCUCUCAGCUGGGGUGUUCA | 1109 | UGAACACCCCAGCUGAGAGCUG |
| DS20-252B | 252 | agctctcagctggggtgttcag | 538 | AGCUCUCAGCUGGGGUGUUCAG | 1110 | CUGAACACCCCAGCUGAGAGCU |
| DS20-253B | 253 | gctctcagctggggtgttcaga | 539 | GCUCUCAGCUGGGGUGUUCAGA | 1111 | UCUGAACACCCCAGCUGAGAGC |
| DS20-254B | 254 | ctctcagctggggtgttcagag | 540 | CUCUCAGCUGGGGUGUUCAGAG | 1112 | CUCUGAACACCCCAGCUGAGAG |
| DS20-255B | 255 | tctcagctggggtgttcagagg | 541 | UCUCAGCUGGGGUGUUCAGAGG | 1113 | CCUCUGAACACCCCAGCUGAGA |
| DS20-256B | 256 | ctcagctggggtgttcagagga | 542 | CUCAGCUGGGGUGUUCAGAGGA | 1114 | UCCUCUGAACACCCCAGCUGAG |
| DS20-257B | 257 | tcagctggggtgttcagaggac | 543 | UCAGCUGGGGUGUUCAGAGGAC | 1115 | GUCCUCUGAACACCCCAGCUGA |
| DS20-258B | 258 | cagctggggtgttcagaggacg | 544 | CAGCUGGGGUGUUCAGAGGACG | 1116 | CGUCCUCUGAACACCCCAGCUG |
| DS20-259B | 259 | agctggggtgttcagaggacgc | 545 | AGCUGGGGUGUUCAGAGGACGC | 1117 | GCGUCCUCUGAACACCCCAGCU |
| DS20-260B | 260 | gctggggtgttcagaggacgcc | 546 | GCUGGGGUGUUCAGAGGACGCC | 1118 | GGCGUCCUCUGAACACCCCAGC |
| DS20-261B | 261 | ctggggtgttcagaggacgcct | 547 | CUGGGGUGUUCAGAGGACGCCU | 1119 | AGGCGUCCUCUGAACACCCCAG |
| DS20-262B | 262 | tggggtgttcagaggacgcctg | 548 | UGGGGUGUUCAGAGGACGCCUG | 1120 | CAGGCGUCCUCUGAACACCCCA |
| DS20-263B | 263 | ggggtgttcagaggacgcctgt | 549 | GGGGUGUUCAGAGGACGCCUGU | 1121 | ACAGGCGUCCUCUGAACACCCC |
| DS20-264B | 264 | gggtgttcagaggacgcctgtg | 550 | GGGUGUUCAGAGGACGCCUGUG | 1122 | CACAGGCGUCCUCUGAACACCC |
| DS20-265B | 265 | ggtgttcagaggacgcctgtgt | 551 | GGUGUUCAGAGGACGCCUGUGU | 1123 | ACACAGGCGUCCUCUGAACACC |
| DS20-266B | 266 | gtgttcagaggacgcctgtgtc | 552 | GUGUUCAGAGGACGCCUGUGUC | 1124 | GACACAGGCGUCCUCUGAACAC |
| DS20-267B | 267 | tgttcagaggacgcctgtgtcc | 553 | UGUUCAGAGGACGCCUGUGUCC | 1125 | GGACACAGGCGUCCUCUGAACA |
| DS20-268B | 268 | gttcagaggacgcctgtgtcct | 554 | GUUCAGAGGACGCCUGUGUCCU | 1126 | AGGACACAGGCGUCCUCUGAAC |
| DS20-269B | 269 | ttcagaggacgcctgtgtcctc | 555 | UUCAGAGGACGCCUGUGUCCUC | 1127 | GAGGACACAGGCGUCCUCUGAA |
| DS20-270B | 270 | tcagaggacgcctgtgtcctcc | 556 | UCAGAGGACGCCUGUGUCCUCC | 1128 | GGAGGACACAGGCGUCCUCUGA |
| DS20-271B | 271 | cagaggacgcctgtgtcctccc | 557 | CAGAGGACGCCUGUGUCCUCCC | 1129 | GGGAGGACACAGGCGUCCUCUG |
| DS20-272B | 272 | agaggacgcctgtgtcctcccc | 558 | AGAGGACGCCUGUGUCCUCCCC | 1130 | GGGGAGGACACAGGCGUCCUCU |
| DS20-273B | 273 | gaggacgcctgtgtcctcccct | 559 | GAGGACGCCUGUGUCCUCCCCU | 1131 | AGGGGAGGACACAGGCGUCCUC |
| DS20-274B | 274 | aggacgcctgtgtcctcccctc | 560 | AGGACGCCUGUGUCCUCCCCUC | 1132 | GAGGGGAGGACACAGGCGUCCU |
| DS20-275B | 275 | ctctgtcacccttggaggcaga | 561 | CUCUGUCACCCUUGGAGGCAGA | 1133 | UCUGCCUCCAAGGGUGACAGAG |
| DS20-276B | 276 | tctgtcacccttggaggcagag | 562 | UCUGUCACCCUUGGAGGCAGAG | 1134 | CUCUGCCUCCAAGGGUGACAGA |
| DS20-277B | 277 | ctgtcacccttggaggcagaga | 563 | CUGUCACCCUUGGAGGCAGAGA | 1135 | UCUCUGCCUCCAAGGGUGACAG |
| DS20-278B | 278 | tgtcacccttggaggcagagaa | 564 | UGUCACCCUUGGAGGCAGAGAA | 1136 | UUCUCUGCCUCCAAGGGUGACA |
| DS20-279B | 279 | gtcacccttggaggcagagaac | 565 | GUCACCCUUGGAGGCAGAGAAC | 1137 | GUUCUCUGCCUCCAAGGGUGAC |
| DS20-280B | 280 | tcacccttggaggcagagaact | 566 | UCACCCUUGGAGGCAGAGAACU | 1138 | AGUUCUCUGCCUCCAAGGGUGA |
| DS20-281B | 281 | cacccttggaggcagagaactt | 567 | CACCCUUGGAGGCAGAGAACUU | 1139 | AAGUUCUCUGCCUCCAAGGGUG |
| DS20-282B | 282 | acccttggaggcagagaacttt | 568 | ACCCUUGGAGGCAGAGAACUUU | 1140 | AAAGUUCUCUGCCUCCAAGGGU |
| DS20-283B | 283 | cccttggaggcagagaactttg | 569 | CCCUUGGAGGCAGAGAACUUUG | 1141 | CAAAGUUCUCUGCCUCCAAGGG |
| DS20-284B | 284 | ccttggaggcagagaactttgc | 570 | CCUUGGAGGCAGAGAACUUUGC | 1142 | GCAAAGUUCUCUGCCUCCAAGG |
| DS20-285B | 285 | cttggaggcagagaactttgcc | 571 | CUUGGAGGCAGAGAACUUUGCC | 1143 | GGCAAAGUUCUCUGCCUCCAAG |
| DS20-286B | 286 | ttggaggcagagaactttgccc | 572 | UUGGAGGCAGAGAACUUUGCCC | 1144 | GGGCAAAGUUCUCUGCCUCCAA |
| DS20-001S | 1 | cgtaagatgtggaccgctggag | 287 | CGUAAGAUGUGGACCGCUGGAG | 1145 | CCAGCGGUCCACAUCUUACG |
| DS20-002S | 2 | gtaagatgtggaccgctggaga | 288 | GUAAGAUGUGGACCGCUGGAGA | 1146 | UCCAGCGGUCCACAUCUUAC |
| DS20-003S | 3 | taagatgtggaccgctggagaa | 289 | UAAGAUGUGGACCGCUGGAGAA | 1147 | CUCCAGCGGUCCACAUCUUA |
| DS20-004S | 4 | aagatgtggaccgctggagaat | 290 | AAGAUGUGGACCGCUGGAGAAU | 1148 | UCUCCAGCGGUCCACAUCUU |
| DS20-005S | 5 | agatgtggaccgctggagaatg | 291 | AGAUGUGGACCGCUGGAGAAUG | 1149 | UUCUCCAGCGGUCCACAUCU |
| DS20-006S | 6 | gatgtggaccgctggagaatgg | 292 | GAUGUGGACCGCUGGAGAAUGG | 1150 | AUUCUCCAGCGGUCCACAUC |
| DS20-007S | 7 | atgtggaccgctggagaatggg | 293 | AUGUGGACCGCUGGAGAAUGGG | 1151 | CAUUCUCCAGCGGUCCACAU |
| DS20-008S | 8 | tgtggaccgctggagaatgggg | 294 | UGUGGACCGCUGGAGAAUGGGG | 1152 | CCAUUCUCCAGCGGUCCACA |
| DS20-009S | 9 | ggggtgctgcctgcagtcaaaa | 295 | GGGGUGCUGCCUGCAGUCAAAA | 1153 | UUGACUGCAGGCAGCACCCC |
| DS20-010S | 10 | gggtgctgcctgcagtcaaaac | 296 | GGGUGCUGCCUGCAGUCAAAAC | 1154 | UUUGACUGCAGGCAGCACCC |
| DS20-011S | 11 | ggtgctgcctgcagtcaaaacg | 297 | GGUGCUGCCUGCAGUCAAAACG | 1155 | UUUUGACUGCAGGCAGCACC |
| DS20-012S | 12 | gtgctgcctgcagtcaaaacgg | 298 | GUGCUGCCUGCAGUCAAAACGG | 1156 | GUUUUGACUGCAGGCAGCAC |
| DS20-013S | 13 | tgctgcctgcagtcaaaacgga | 299 | UGCUGCCUGCAGUCAAAACGGA | 1157 | CGUUUUGACUGCAGGCAGCA |
| DS20-014S | 14 | gctgcctgcagtcaaaacggag | 300 | GCUGCCUGCAGUCAAAACGGAG | 1158 | CCGUUUUGACUGCAGGCAGC |
| DS20-015S | 15 | ctgcctgcagtcaaaacggagt | 301 | CUGCCUGCAGUCAAAACGGAGU | 1159 | UCCGUUUUGACUGCAGGCAG |
| DS20-016S | 16 | tgcctgcagtcaaaacggagtg | 302 | UGCCUGCAGUCAAAACGGAGUG | 1160 | CUCCGUUUUGACUGCAGGCA |
| DS20-017S | 17 | gcctgcagtcaaaacggagtgg | 303 | GCCUGCAGUCAAAACGGAGUGG | 1161 | ACUCCGUUUUGACUGCAGGC |
| DS20-018S | 18 | cctgcagtcaaaacggagtggg | 304 | CCUGCAGUCAAAACGGAGUGGG | 1162 | CACUCCGUUUUGACUGCAGG |
| DS20-019S | 19 | ctgcagtcaaaacggagtgggg | 305 | CUGCAGUCAAAACGGAGUGGGG | 1163 | CCACUCCGUUUUGACUGCAG |
| DS20-020S | 20 | ggtgcccagctcagggccagaa | 306 | GGUGCCCAGCUCAGGGCCAGAA | 1164 | CUGGCCCUGAGCUGGGCACC |
| DS20-021S | 21 | gtgcccagctcagggccagaat | 307 | GUGCCCAGCUCAGGGCCAGAAU | 1165 | UCUGGCCCUGAGCUGGGCAC |
| DS20-022S | 22 | tgcccagctcagggccagaatg | 308 | UGCCCAGCUCAGGGCCAGAAUG | 1166 | UUCUGGCCCUGAGCUGGGCA |
| DS20-023S | 23 | gcccagctcagggccagaatga | 309 | GCCCAGCUCAGGGCCAGAAUGA | 1167 | AUUCUGGCCCUGAGCUGGGC |
| DS20-024S | 24 | cccagctcagggccagaatgat | 310 | CCCAGCUCAGGGCCAGAAUGAU | 1168 | CAUUCUGGCCCUGAGCUGGG |
| DS20-025S | 25 | ccagctcagggccagaatgatc | 311 | CCAGCUCAGGGCCAGAAUGAUC | 1169 | UCAUUCUGGCCCUGAGCUGG |
| DS20-026S | 26 | cagctcagggccagaatgatcc | 312 | CAGCUCAGGGCCAGAAUGAUCC | 1170 | AUCAUUCUGGCCCUGAGCUG |
| DS20-027S | 27 | agctcagggccagaatgatcct | 313 | AGCUCAGGGCCAGAAUGAUCCU | 1171 | GAUCAUUCUGGCCCUGAGCU |
| DS20-028S | 28 | gctcagggccagaatgatccta | 314 | GCUCAGGGCCAGAAUGAUCCUA | 1172 | GGAUCAUUCUGGCCCUGAGC |
| DS20-029S | 29 | ctcagggccagaatgatcctat | 315 | CUCAGGGCCAGAAUGAUCCUAU | 1173 | AGGAUCAUUCUGGCCCUGAG |
| DS20-030S | 30 | tcagggccagaatgatcctatt | 316 | UCAGGGCCAGAAUGAUCCUAUU | 1174 | UAGGAUCAUUCUGGCCCUGA |
| DS20-031S | 31 | cagggccagaatgatcctattc | 317 | CAGGGCCAGAAUGAUCCUAUUC | 1175 | AUAGGAUCAUUCUGGCCCUG |
| DS20-032S | 32 | agggccagaatgatcctattcc | 318 | AGGGCCAGAAUGAUCCUAUUCC | 1176 | AAUAGGAUCAUUCUGGCCCU |
| DS20-033S | 33 | gggccagaatgatcctattccc | 319 | GGGCCAGAAUGAUCCUAUUCCC | 1177 | GAAUAGGAUCAUUCUGGCCC |
| DS20-034S | 34 | ggccagaatgatcctattcccg | 320 | GGCCAGAAUGAUCCUAUUCCCG | 1178 | GGAAUAGGAUCAUUCUGGCC |
| DS20-035S | 35 | gccagaatgatcctattcccgg | 321 | GCCAGAAUGAUCCUAUUCCCGG | 1179 | GGGAAUAGGAUCAUUCUGGC |
| DS20-036S | 36 | ccagaatgatcctattcccggc | 322 | CCAGAAUGAUCCUAUUCCCGGC | 1180 | CGGGAAUAGGAUCAUUCUGG |
| DS20-037S | 37 | cagaatgatcctattcccggca | 323 | CAGAAUGAUCCUAUUCCCGGCA | 1181 | CCGGGAAUAGGAUCAUUCUG |
| DS20-038S | 38 | agaatgatcctattcccggcac | 324 | AGAAUGAUCCUAUUCCCGGCAC | 1182 | GCCGGGAAUAGGAUCAUUCU |
| DS20-039S | 39 | gaatgatcctattcccggcact | 325 | GAAUGAUCCUAUUCCCGGCACU | 1183 | UGCCGGGAAUAGGAUCAUUC |
| DS20-040S | 40 | aatgatcctattcccggcactt | 326 | AAUGAUCCUAUUCCCGGCACUU | 1184 | GUGCCGGGAAUAGGAUCAUU |
| DS20-041S | 41 | atgatcctattcccggcacttc | 327 | AUGAUCCUAUUCCCGGCACUUC | 1185 | AGUGCCGGGAAUAGGAUCAU |
| DS20-042S | 42 | tgatcctattcccggcacttct | 328 | UGAUCCUAUUCCCGGCACUUCU | 1186 | AAGUGCCGGGAAUAGGAUCA |
| DS20-043S | 43 | gatcctattcccggcacttctc | 329 | GAUCCUAUUCCCGGCACUUCUC | 1187 | GAAGUGCCGGGAAUAGGAUC |
| DS20-044S | 44 | atcctattcccggcacttctca | 330 | AUCCUAUUCCCGGCACUUCUCA | 1188 | AGAAGUGCCGGGAAUAGGAU |
| DS20-045S | 45 | tcctattcccggcacttctcag | 331 | UCCUAUUCCCGGCACUUCUCAG | 1189 | GAGAAGUGCCGGGAAUAGGA |
| DS20-046S | 46 | cctattcccggcacttctcagt | 332 | CCUAUUCCCGGCACUUCUCAGU | 1190 | UGAGAAGUGCCGGGAAUAGG |
| DS20-047S | 47 | ctattcccggcacttctcagtg | 333 | CUAUUCCCGGCACUUCUCAGUG | 1191 | CUGAGAAGUGCCGGGAAUAG |
| DS20-048S | 48 | tattcccggcacttctcagtga | 334 | UAUUCCCGGCACUUCUCAGUGA | 1192 | ACUGAGAAGUGCCGGGAAUA |
| DS20-049S | 49 | attcccggcacttctcagtgag | 335 | AUUCCCGGCACUUCUCAGUGAG | 1193 | CACUGAGAAGUGCCGGGAAU |
| DS20-050S | 50 | ttoccggcacttctcagtgagg | 336 | UUCCCGGCACUUCUCAGUGAGG | 1194 | UCACUGAGAAGUGCCGGGAA |
| DS20-051S | 51 | tcccggcacttctcagtgaggc | 337 | UCCCGGCACUUCUCAGUGAGGC | 1195 | CUCACUGAGAAGUGCCGGGA |
| DS20-052S | 52 | cccggcacttctcagtgaggct | 338 | CCCGGCACUUCUCAGUGAGGCU | 1196 | CCUCACUGAGAAGUGCCGGG |
| DS20-053S | 53 | ccggcacttctcagtgaggctc | 339 | CCGGCACUUCUCAGUGAGGCUC | 1197 | GCCUCACUGAGAAGUGCCGG |
| DS20-054S | 54 | cggcacttctcagtgaggctct | 340 | CGGCACUUCUCAGUGAGGCUCU | 1198 | AGCCUCACUGAGAAGUGCCG |
| DS20-055S | 55 | ggcacttctcagtgaggctctg | 341 | GGCACUUCUCAGUGAGGCUCUG | 1199 | GAGCCUCACUGAGAAGUGCC |
| DS20-056S | 56 | gcacttctcagtgaggctctgt | 342 | GCACUUCUCAGUGAGGCUCUGU | 1200 | AGAGCCUCACUGAGAAGUGC |
| DS20-057S | 57 | cacttctcagtgaggctctgtg | 343 | CACUUCUCAGUGAGGCUCUGUG | 1201 | CAGAGCCUCACUGAGAAGUG |
| DS20-058S | 58 | acttctcagtgaggctctgtgg | 344 | ACUUCUCAGUGAGGCUCUGUGG | 1202 | ACAGAGCCUCACUGAGAAGU |
| DS20-059S | 59 | cttctcagtgaggctctgtggc | 345 | CUUCUCAGUGAGGCUCUGUGGC | 1203 | CACAGAGCCUCACUGAGAAG |
| DS20-060S | 60 | ttctcagtgaggctctgtggct | 346 | UUCUCAGUGAGGCUCUGUGGCU | 1204 | CCACAGAGCCUCACUGAGAA |
| DS20-061S | 61 | tctcagtgaggctctgtggctc | 347 | UCUCAGUGAGGCUCUGUGGCUC | 1205 | GCCACAGAGCCUCACUGAGA |
| DS20-062S | 62 | ctcagtgaggctctgtggctca | 348 | CUCAGUGAGGCUCUGUGGCUCA | 1206 | AGCCACAGAGCCUCACUGAG |
| DS20-063S | 63 | tcagtgaggctctgtggctcac | 349 | UCAGUGAGGCUCUGUGGCUCAC | 1207 | GAGCCACAGAGCCUCACUGA |
| DS20-064S | 64 | cagtgaggctctgtggctcacc | 350 | CAGUGAGGCUCUGUGGCUCACC | 1208 | UGAGCCACAGAGCCUCACUG |
| DS20-065S | 65 | agtgaggctctgtggctcacct | 351 | AGUGAGGCUCUGUGGCUCACCU | 1209 | GUGAGCCACAGAGCCUCACU |
| DS20-066S | 66 | gtgaggctctgtggctcaccta | 352 | GUGAGGCUCUGUGGCUCACCUA | 1210 | GGUGAGCCACAGAGCCUCAC |
| DS20-067S | 67 | tgaggctctgtggctcacctaa | 353 | UGAGGCUCUGUGGCUCACCUAA | 1211 | AGGUGAGCCACAGAGCCUCA |
| DS20-068S | 68 | gaggctctgtggctcacctaag | 354 | GAGGCUCUGUGGCUCACCUAAG | 1212 | UAGGUGAGCCACAGAGCCUC |
| DS20-069S | 69 | aggctctgtggctcacctaaga | 355 | AGGCUCUGUGGCUCACCUAAGA | 1213 | UUAGGUGAGCCACAGAGCCU |
| DS20-070S | 70 | ggctctgtggctcacctaagaa | 356 | GGCUCUGUGGCUCACCUAAGAA | 1214 | CUUAGGUGAGCCACAGAGCC |
| DS20-071S | 71 | gctctgtggctcacctaagaaa | 357 | GCUCUGUGGCUCACCUAAGAAA | 1215 | UCUUAGGUGAGCCACAGAGC |
| DS20-072S | 72 | ctctgtggctcacctaagaaac | 358 | CUCUGUGGCUCACCUAAGAAAC | 1216 | UUCUUAGGUGAGCCACAGAG |
| DS20-073S | 73 | tctgtggctcacctaagaaacc | 359 | UCUGUGGCUCACCUAAGAAACC | 1217 | UUUCUUAGGUGAGCCACAGA |
| DS20-074S | 74 | ctgtggctcacctaagaaacca | 360 | CUGUGGCUCACCUAAGAAACCA | 1218 | GUUUCUUAGGUGAGCCACAG |
| DS20-075S | 75 | tgtggctcacctaagaaaccag | 361 | UGUGGCUCACCUAAGAAACCAG | 1219 | GGUUUCUUAGGUGAGCCACA |
| DS20-076S | 76 | gtggctcacctaagaaaccagc | 362 | GUGGCUCACCUAAGAAACCAGC | 1220 | UGGUUUCUUAGGUGAGCCAC |
| DS20-077S | 77 | tggctcacctaagaaaccagcc | 363 | UGGCUCACCUAAGAAACCAGCC | 1221 | CUGGUUUCUUAGGUGAGCCA |
| DS20-078S | 78 | ggctcacctaagaaaccagcct | 364 | GGCUCACCUAAGAAACCAGCCU | 1222 | GCUGGUUUCUUAGGUGAGCC |
| DS20-079S | 79 | gctcacctaagaaaccagcctc | 365 | GCUCACCUAAGAAACCAGCCUC | 1223 | GGCUGGUUUCUUAGGUGAGC |
| DS20-080S | 80 | ctcacctaagaaaccagcctcc | 366 | CUCACCUAAGAAACCAGCCUCC | 1224 | AGGCUGGUUUCUUAGGUGAG |
| DS20-081S | 81 | tcacctaagaaaccagcctccc | 367 | UCACCUAAGAAACCAGCCUCCC | 1225 | GAGGCUGGUUUCUUAGGUGA |
| DS20-082S | 82 | cacctaagaaaccagcctccct | 368 | CACCUAAGAAACCAGCCUCCCU | 1226 | GGAGGCUGGUUUCUUAGGUG |
| DS20-083S | 83 | acctaagaaaccagcctccctt | 369 | ACCUAAGAAACCAGCCUCCCUU | 1227 | GGGAGGCUGGUUUCUUAGGU |
| DS20-084S | 84 | cctaagaaaccagcctcccttg | 370 | CCUAAGAAACCAGCCUCCCUUG | 1228 | AGGGAGGCUGGUUUCUUAGG |
| DS20-085S | 85 | ctaagaaaccagcctcccttgc | 371 | CUAAGAAACCAGCCUCCCUUGC | 1229 | AAGGGAGGCUGGUUUCUUAG |
| DS20-086S | 86 | taagaaaccagcctcccttgca | 372 | UAAGAAACCAGCCUCCCUUGCA | 1230 | CAAGGGAGGCUGGUUUCUUA |
| DS20-087S | 87 | aagaaaccagcctcccttgcag | 373 | AAGAAACCAGCCUCCCUUGCAG | 1231 | GCAAGGGAGGCUGGUUUCUU |
| DS20-088S | 88 | agaaaccagcctcccttgcagg | 374 | AGAAACCAGCCUCCCUUGCAGG | 1232 | UGCAAGGGAGGCUGGUUUCU |
| DS20-089S | 89 | gaaaccagcctcccttgcaggc | 375 | GAAACCAGCCUCCCUUGCAGGC | 1233 | CUGCAAGGGAGGCUGGUUUC |
| DS20-090S | 90 | aaaccagcctcccttgcaggca | 376 | AAACCAGCCUCCCUUGCAGGCA | 1234 | CCUGCAAGGGAGGCUGGUUU |
| DS20-091S | 91 | aaccagcctcccttgcaggcaa | 377 | AACCAGCCUCCCUUGCAGGCAA | 1235 | GCCUGCAAGGGAGGCUGGUU |
| DS20-092S | 92 | accagcctcccttgcaggcaac | 378 | ACCAGCCUCCCUUGCAGGCAAC | 1236 | UGCCUGCAAGGGAGGCUGGU |
| DS20-093S | 93 | ccagcctcccttgcaggcaacg | 379 | CCAGCCUCCCUUGCAGGCAACG | 1237 | UUGCCUGCAAGGGAGGCUGG |
| DS20-094S | 94 | cagcctcccttgcaggcaacgg | 380 | CAGCCUCCCUUGCAGGCAACGG | 1238 | GUUGCCUGCAAGGGAGGCUG |
| DS20-095S | 95 | agcctcccttgcaggcaacggc | 381 | AGCCUCCCUUGCAGGCAACGGC | 1239 | CGUUGCCUGCAAGGGAGGCU |
| DS20-096S | 96 | cctcccttgcaggcaacggcct | 382 | CCUCCCUUGCAGGCAACGGCCU | 1240 | GCCGUUGCCUGCAAGGGAGG |
| DS20-097S | 97 | ctcccttgcaggcaacggccta | 383 | CUCCCUUGCAGGCAACGGCCUA | 1241 | GGCCGUUGCCUGCAAGGGAG |
| DS20-098S | 98 | tcccttgcaggcaacggcctag | 384 | UCCCUUGCAGGCAACGGCCUAG | 1242 | AGGCCGUUGCCUGCAAGGGA |
| DS20-099S | 99 | cccttgcaggcaacggcctagc | 385 | CCCUUGCAGGCAACGGCCUAGC | 1243 | UAGGCCGUUGCCUGCAAGGG |
| DS20-100S | 100 | ccttgcaggcaacggcctagct | 386 | CCUUGCAGGCAACGGCCUAGCU | 1244 | CUAGGCCGUUGCCUGCAAGG |
| DS20-101S | 101 | cttgcaggcaacggcctagctg | 387 | CUUGCAGGCAACGGCCUAGCUG | 1245 | GCUAGGCCGUUGCCUGCAAG |
| DS20-102S | 102 | ttgcaggcaacggcctagctgg | 388 | UUGCAGGCAACGGCCUAGCUGG | 1246 | AGCUAGGCCGUUGCCUGCAA |
| DS20-103S | 103 | tgcaggcaacggcctagctggc | 389 | UGCAGGCAACGGCCUAGCUGGC | 1247 | CAGCUAGGCCGUUGCCUGCA |
| DS20-104S | 104 | caggcaacggcctagctggcct | 390 | CAGGCAACGGCCUAGCUGGCCU | 1248 | GCCAGCUAGGCCGUUGCCUG |
| DS20-105S | 105 | aggcaacggcctagctggcctg | 391 | AGGCAACGGCCUAGCUGGCCUG | 1249 | GGCCAGCUAGGCCGUUGCCU |
| DS20-106S | 106 | gcaacggcctagctggcctggt | 392 | GCAACGGCCUAGCUGGCCUGGU | 1250 | CAGGCCAGCUAGGCCGUUGC |
| DS20-107S | 107 | caacggcctagctggcctggtc | 393 | CAACGGCCUAGCUGGCCUGGUC | 1251 | CCAGGCCAGCUAGGCCGUUG |
| DS20-108S | 108 | aacggcctagctggcctggtct | 394 | AACGGCCUAGCUGGCCUGGUCU | 1252 | ACCAGGCCAGCUAGGCCGUU |
| DS20-109S | 109 | acggcctagctggcctggtctg | 395 | ACGGCCUAGCUGGCCUGGUCUG | 1253 | GACCAGGCCAGCUAGGCCGU |
| DS20-110S | 110 | ggcctagctggcctggtctgga | 396 | GGCCUAGCUGGCCUGGUCUGGA | 1254 | CAGACCAGGCCAGCUAGGCC |
| DS20-111S | 111 | gcctagctggcctggtctggag | 397 | GCCUAGCUGGCCUGGUCUGGAG | 1255 | CCAGACCAGGCCAGCUAGGC |
| DS20-112S | 112 | cctagctggcctggtctggagg | 398 | CCUAGCUGGCCUGGUCUGGAGG | 1256 | UCCAGACCAGGCCAGCUAGG |
| DS20-113S | 113 | ctagctggcctggtctggaggc | 399 | CUAGCUGGCCUGGUCUGGAGGC | 1257 | CUCCAGACCAGGCCAGCUAG |
| DS20-114S | 114 | tagctggcctggtctggaggct | 400 | UAGCUGGCCUGGUCUGGAGGCU | 1258 | CCUCCAGACCAGGCCAGCUA |
| DS20-115S | 115 | agctggcctggtctggaggctc | 401 | AGCUGGCCUGGUCUGGAGGCUC | 1259 | GCCUCCAGACCAGGCCAGCU |
| DS20-116S | 116 | gctggcctggtctggaggctct | 402 | GCUGGCCUGGUCUGGAGGCUCU | 1260 | AGCCUCCAGACCAGGCCAGC |
| DS20-117S | 117 | ctggcctggtctggaggctctc | 403 | CUGGCCUGGUCUGGAGGCUCUC | 1261 | GAGCCUCCAGACCAGGCCAG |
| DS20-118S | 118 | tttacatccacacccaagatac | 404 | UUUACAUCCACACCCAAGAUAC | 1262 | AUCUUGGGUGUGGAUGUAAA |
| DS20-119S | 119 | tcttgagatttgactcgcatga | 405 | UCUUGAGAUUUGACUCGCAUGA | 1263 | AUGCGAGUCAAAUCUCAAGA |
| DS20-120S | 120 | cttgagatttgactcgcatgat | 406 | CUUGAGAUUUGACUCGCAUGAU | 1264 | CAUGCGAGUCAAAUCUCAAG |
| DS20-121S | 121 | tgagatttgactcgcatgattg | 407 | UGAGAUUUGACUCGCAUGAUUG | 1265 | AUCAUGCGAGUCAAAUCUCA |
| DS20-122S | 122 | gagatttgactcgcatgattgc | 408 | GAGAUUUGACUCGCAUGAUUGC | 1266 | AAUCAUGCGAGUCAAAUCUC |
| DS20-123S | 123 | agatttgactcgcatgattgct | 409 | AGAUUUGACUCGCAUGAUUGCU | 1267 | CAAUCAUGCGAGUCAAAUCU |
| DS20-124S | 124 | gatttgactcgcatgattgcta | 410 | GAUUUGACUCGCAUGAUUGCUA | 1268 | GCAAUCAUGCGAGUCAAAUC |
| DS20-125S | 125 | tttgactcgcatgattgctatg | 411 | UUUGACUCGCAUGAUUGCUAUG | 1269 | UAGCAAUCAUGCGAGUCAAA |
| DS20-126S | 126 | ttgactcgcatgattgctatgg | 412 | UUGACUCGCAUGAUUGCUAUGG | 1270 | AUAGCAAUCAUGCGAGUCAA |
| DS20-127S | 127 | tgactcgcatgattgctatggg | 413 | UGACUCGCAUGAUUGCUAUGGG | 1271 | CAUAGCAAUCAUGCGAGUCA |
| DS20-128S | 128 | gactcgcatgattgctatggga | 414 | GACUCGCAUGAUUGCUAUGGGA | 1272 | CCAUAGCAAUCAUGCGAGUC |
| DS20-129S | 129 | actcgcatgattgctatgggac | 415 | ACUCGCAUGAUUGCUAUGGGAC | 1273 | CCCAUAGCAAUCAUGCGAGU |
| DS20-130S | 130 | ctcgcatgattgctatgggaca | 416 | CUCGCAUGAUUGCUAUGGGACA | 1274 | UCCCAUAGCAAUCAUGCGAG |
| DS20-131S | 131 | tcgcatgattgctatgggacaa | 417 | UCGCAUGAUUGCUAUGGGACAA | 1275 | GUCCCAUAGCAAUCAUGCGA |
| DS20-132S | 132 | cgcatgattgctatgggacaag | 418 | CGCAUGAUUGCUAUGGGACAAG | 1276 | UGUCCCAUAGCAAUCAUGCG |
| DS20-133S | 133 | gcatgattgctatgggacaagt | 419 | GCAUGAUUGCUAUGGGACAAGU | 1277 | UUGUCCCAUAGCAAUCAUGC |
| DS20-134S | 134 | catgattgctatgggacaagtt | 420 | CAUGAUUGCUAUGGGACAAGUU | 1278 | CUUGUCCCAUAGCAAUCAUG |
| DS20-135S | 135 | gattgctatgggacaagttttc | 421 | GAUUGCUAUGGGACAAGUUUUC | 1279 | AAACUUGUCCCAUAGCAAUC |
| DS20-136S | 136 | tgctatgggacaagttttcatc | 422 | UGCUAUGGGACAAGUUUUCAUC | 1280 | UGAAAACUUGUCCCAUAGCA |
| DS20-137S | 137 | gctatgggacaagttttcatct | 423 | GCUAUGGGACAAGUUUUCAUCU | 1281 | AUGAAAACUUGUCCCAUAGC |
| DS20-138S | 138 | ctatgggacaagttttcatctg | 424 | CUAUGGGACAAGUUUUCAUCUG | 1282 | GAUGAAAACUUGUCCCAUAG |
| DS20-139S | 139 | tatgggacaagttttcatctgc | 425 | UAUGGGACAAGUUUUCAUCUGC | 1283 | AGAUGAAAACUUGUCCCAUA |
| DS20-140S | 140 | atgggacaagttttcatctgca | 426 | AUGGGACAAGUUUUCAUCUGCA | 1284 | CAGAUGAAAACUUGUCCCAU |
| DS20-141S | 141 | tgggacaagttttcatctgcag | 427 | UGGGACAAGUUUUCAUCUGCAG | 1285 | GCAGAUGAAAACUUGUCCCA |
| DS20-142S | 142 | gggacaagttttcatctgcagt | 428 | GGGACAAGUUUUCAUCUGCAGU | 1286 | UGCAGAUGAAAACUUGUCCC |
| DS20-143S | 143 | ggacaagttttcatctgcagtt | 429 | GGACAAGUUUUCAUCUGCAGUU | 1287 | CUGCAGAUGAAAACUUGUCC |
| DS20-144S | 144 | ctgcagtttaaatctgtttccc | 430 | CUGCAGUUUAAAUCUGUUUCCC | 1288 | GAAACAGAUUUAAACUGCAG |
| DS20-145S | 145 | taggggtttggaattctagatc | 431 | UAGGGGUUUGGAAUUCUAGAUC | 1289 | UCUAGAAUUCCAAACCCCUA |
| DS20-146S | 146 | aggggtttggaattctagatcg | 432 | AGGGGUUUGGAAUUCUAGAUCG | 1290 | AUCUAGAAUUCCAAACCCCU |
| DS20-147S | 147 | ggggtttggaattctagatcgt | 433 | GGGGUUUGGAAUUCUAGAUCGU | 1291 | GAUCUAGAAUUCCAAACCCC |
| DS20-148S | 148 | gggtttggaattctagatcgta | 434 | GGGUUUGGAAUUCUAGAUCGUA | 1292 | CGAUCUAGAAUUCCAAACCC |
| DS20-149S | 149 | gatcgtatttgaagtgttggtg | 435 | GAUCGUAUUUGAAGUGUUGGUG | 1293 | CCAACACUUCAAAUACGAUC |
| DS20-150S | 150 | atcgtatttgaagtgttggtgc | 436 | AUCGUAUUUGAAGUGUUGGUGC | 1294 | ACCAACACUUCAAAUACGAU |
| DS20-151S | 151 | tcgtatttgaagtgttggtgcc | 437 | UCGUAUUUGAAGUGUUGGUGCC | 1295 | CACCAACACUUCAAAUACGA |
| DS20-152S | 152 | cgtatttgaagtgttggtgcca | 438 | CGUAUUUGAAGUGUUGGUGCCA | 1296 | GCACCAACACUUCAAAUACG |
| DS20-153S | 153 | gtatttgaagtgttggtgccac | 439 | GUAUUUGAAGUGUUGGUGCCAC | 1297 | GGCACCAACACUUCAAAUAC |
| DS20-154S | 154 | tatttgaagtgttggtgccaca | 440 | UAUUUGAAGUGUUGGUGCCACA | 1298 | UGGCACCAACACUUCAAAUA |
| DS20-155S | 155 | atttgaagtgttggtgccacac | 441 | AUUUGAAGUGUUGGUGCCACAC | 1299 | GUGGCACCAACACUUCAAAU |
| DS20-156S | 156 | cacaccttaacacctgcacgct | 442 | CACACCUUAACACCUGCACGCU | 1300 | CGUGCAGGUGUUAAGGUGUG |
| DS20-157S | 157 | acaccttaacacctgcacgctg | 443 | ACACCUUAACACCUGCACGCUG | 1301 | GCGUGCAGGUGUUAAGGUGU |
| DS20-158S | 158 | caccttaacacctgcacgctgg | 444 | CACCUUAACACCUGCACGCUGG | 1302 | AGCGUGCAGGUGUUAAGGUG |
| DS20-159S | 159 | accttaacacctgcacgctggc | 445 | ACCUUAACACCUGCACGCUGGC | 1303 | CAGCGUGCAGGUGUUAAGGU |
| DS20-160S | 160 | ccttaacacctgcacgctggca | 446 | CCUUAACACCUGCACGCUGGCA | 1304 | CCAGCGUGCAGGUGUUAAGG |
| DS20-161S | 161 | cttaacacctgcacgctggcaa | 447 | CUUAACACCUGCACGCUGGCAA | 1305 | GCCAGCGUGCAGGUGUUAAG |
| DS20-162S | 162 | ttaacacctgcacgctggcaac | 448 | UUAACACCUGCACGCUGGCAAC | 1306 | UGCCAGCGUGCAGGUGUUAA |
| DS20-163S | 163 | taacacctgcacgctggcaaca | 449 | UAACACCUGCACGCUGGCAACA | 1307 | UUGCCAGCGUGCAGGUGUUA |
| DS20-164S | 164 | aacacctgcacgctggcaacaa | 450 | AACACCUGCACGCUGGCAACAA | 1308 | GUUGCCAGCGUGCAGGUGUU |
| DS20-165S | 165 | acacctgcacgctggcaacaaa | 451 | ACACCUGCACGCUGGCAACAAA | 1309 | UGUUGCCAGCGUGCAGGUGU |
| DS20-166S | 166 | cacctgcacgctggcaacaaaa | 452 | CACCUGCACGCUGGCAACAAAA | 1310 | UUGUUGCCAGCGUGCAGGUG |
| DS20-167S | 167 | acctgcacgctggcaacaaaac | 453 | ACCUGCACGCUGGCAACAAAAC | 1311 | UUUGUUGCCAGCGUGCAGGU |
| DS20-168S | 168 | cctgcacgctggcaacaaaacc | 454 | CCUGCACGCUGGCAACAAAACC | 1312 | UUUUGUUGCCAGCGUGCAGG |
| DS20-169S | 169 | ctgcacgctggcaacaaaaccg | 455 | CUGCACGCUGGCAACAAAACCG | 1313 | GUUUUGUUGCCAGCGUGCAG |
| DS20-170S | 170 | tgcacgctggcaacaaaaccgt | 456 | UGCACGCUGGCAACAAAACCGU | 1314 | GGUUUUGUUGCCAGCGUGCA |
| DS20-171S | 171 | gcacgctggcaacaaaaccgtc | 457 | GCACGCUGGCAACAAAACCGUC | 1315 | CGGUUUUGUUGCCAGCGUGC |
| DS20-172S | 172 | cacgctggcaacaaaaccgtcc | 458 | CACGCUGGCAACAAAACCGUCC | 1316 | ACGGUUUUGUUGCCAGCGUG |
| DS20-173S | 173 | acgctggcaacaaaaccgtccg | 459 | ACGCUGGCAACAAAACCGUCCG | 1317 | GACGGUUUUGUUGCCAGCGU |
| DS20-174S | 174 | cgctggcaacaaaaccgtccgc | 460 | CGCUGGCAACAAAACCGUCCGC | 1318 | GGACGGUUUUGUUGCCAGCG |
| DS20-175S | 175 | gctggcaacaaaaccgtccgct | 461 | GCUGGCAACAAAACCGUCCGCU | 1319 | CGGACGGUUUUGUUGCCAGC |
| DS20-176S | 176 | ctggcaacaaaaccgtccgctc | 462 | CUGGCAACAAAACCGUCCGCUC | 1320 | GCGGACGGUUUUGUUGCCAG |
| DS20-177S | 177 | tggcaacaaaaccgtccgctct | 463 | UGGCAACAAAACCGUCCGCUCU | 1321 | AGCGGACGGUUUUGUUGCCA |
| DS20-178S | 178 | ggcaacaaaaccgtccgctctg | 464 | GGCAACAAAACCGUCCGCUCUG | 1322 | GAGCGGACGGUUUUGUUGCC |
| DS20-179S | 179 | gcaacaaaaccgtccgctctgc | 465 | GCAACAAAACCGUCCGCUCUGC | 1323 | AGAGCGGACGGUUUUGUUGC |
| DS20-180S | 180 | caacaaaaccgtccgctctgca | 466 | CAACAAAACCGUCCGCUCUGCA | 1324 | CAGAGCGGACGGUUUUGUUG |
| DS20-181S | 181 | aacaaaaccgtccgctctgcag | 467 | AACAAAACCGUCCGCUCUGCAG | 1325 | GCAGAGCGGACGGUUUUGUU |
| DS20-182S | 182 | acaaaaccgtccgctctgcagc | 468 | ACAAAACCGUCCGCUCUGCAGC | 1326 | UGCAGAGCGGACGGUUUUGU |
| DS20-183S | 183 | caaaaccgtccgctctgcagca | 469 | CAAAACCGUCCGCUCUGCAGCA | 1327 | CUGCAGAGCGGACGGUUUUG |
| DS20-184S | 184 | aaaaccgtccgctctgcagcac | 470 | AAAACCGUCCGCUCUGCAGCAC | 1328 | GCUGCAGAGCGGACGGUUUU |
| DS20-185S | 185 | aaaccgtccgctctgcagcaca | 471 | AAACCGUCCGCUCUGCAGCACA | 1329 | UGCUGCAGAGCGGACGGUUU |
| DS20-186S | 186 | aaccgtccgctctgcagcacag | 472 | AACCGUCCGCUCUGCAGCACAG | 1330 | GUGCUGCAGAGCGGACGGUU |
| DS20-187S | 187 | accgtccgctctgcagcacagc | 473 | ACCGUCCGCUCUGCAGCACAGC | 1331 | UGUGCUGCAGAGCGGACGGU |
| DS20-188S | 188 | ccgtccgctctgcagcacagct | 474 | CCGUCCGCUCUGCAGCACAGCU | 1332 | CUGUGCUGCAGAGCGGACGG |
| DS20-189S | 189 | cgtccgctctgcagcacagctg | 475 | CGUCCGCUCUGCAGCACAGCUG | 1333 | GCUGUGCUGCAGAGCGGACG |
| DS20-190S | 190 | gtccgctctgcagcacagctgg | 476 | GUCCGCUCUGCAGCACAGCUGG | 1334 | AGCUGUGCUGCAGAGCGGAC |
| DS20-191S | 191 | tccgctctgcagcacagctggg | 477 | UCCGCUCUGCAGCACAGCUGGG | 1335 | CAGCUGUGCUGCAGAGCGGA |
| DS20-192S | 192 | cgctctgcagcacagctggggt | 478 | CGCUCUGCAGCACAGCUGGGGU | 1336 | CCCAGCUGUGCUGCAGAGCG |
| DS20-193S | 193 | gctctgcagcacagctggggtc | 479 | GCUCUGCAGCACAGCUGGGGUC | 1337 | CCCCAGCUGUGCUGCAGAGC |
| DS20-194S | 194 | ctctgcagcacagctggggtca | 480 | CUCUGCAGCACAGCUGGGGUCA | 1338 | ACCCCAGCUGUGCUGCAGAG |
| DS20-195S | 195 | tctgcagcacagctggggtcac | 481 | UCUGCAGCACAGCUGGGGUCAC | 1339 | GACCCCAGCUGUGCUGCAGA |
| DS20-196S | 196 | ctgcagcacagctggggtcacc | 482 | CUGCAGCACAGCUGGGGUCACC | 1340 | UGACCCCAGCUGUGCUGCAG |
| DS20-197S | 197 | tgcagcacagctggggtcacct | 483 | UGCAGCACAGCUGGGGUCACCU | 1341 | GUGACCCCAGCUGUGCUGCA |
| DS20-198S | 198 | gcagcacagctggggtcacctg | 484 | GCAGCACAGCUGGGGUCACCUG | 1342 | GGUGACCCCAGCUGUGCUGC |
| DS20-199S | 199 | cagcacagctggggtcacctga | 485 | CAGCACAGCUGGGGUCACCUGA | 1343 | AGGUGACCCCAGCUGUGCUG |
| DS20-200S | 200 | agcacagctggggtcacctgac | 486 | AGCACAGCUGGGGUCACCUGAC | 1344 | CAGGUGACCCCAGCUGUGCU |
| DS20-201S | 201 | gcacagctggggtcacctgacc | 487 | GCACAGCUGGGGUCACCUGACC | 1345 | UCAGGUGACCCCAGCUGUGC |
| DS20-202S | 202 | cacagctggggtcacctgacct | 488 | CACAGCUGGGGUCACCUGACCU | 1346 | GUCAGGUGACCCCAGCUGUG |
| DS20-203S | 203 | acagctggggtcacctgacctt | 489 | ACAGCUGGGGUCACCUGACCUU | 1347 | GGUCAGGUGACCCCAGCUGU |
| DS20-204S | 204 | cagctggggtcacctgaccttt | 490 | CAGCUGGGGUCACCUGACCUUU | 1348 | AGGUCAGGUGACCCCAGCUG |
| DS20-205S | 205 | agctggggtcacctgacctttc | 491 | AGCUGGGGUCACCUGACCUUUC | 1349 | AAGGUCAGGUGACCCCAGCU |
| DS20-206S | 206 | gctggggtcacctgacctttct | 492 | GCUGGGGUCACCUGACCUUUCU | 1350 | AAAGGUCAGGUGACCCCAGC |
| DS20-207S | 207 | ctggggtcacctgacctttctc | 493 | CUGGGGUCACCUGACCUUUCUC | 1351 | GAAAGGUCAGGUGACCCCAG |
| DS20-208S | 208 | tggggtcacctgacctttctcc | 494 | UGGGGUCACCUGACCUUUCUCC | 1352 | AGAAAGGUCAGGUGACCCCA |
| DS20-209S | 209 | ggggtcacctgacctttctcct | 495 | GGGGUCACCUGACCUUUCUCCU | 1353 | GAGAAAGGUCAGGUGACCCC |
| DS20-210S | 210 | gggtcacctgacctttctcctg | 496 | GGGUCACCUGACCUUUCUCCUG | 1354 | GGAGAAAGGUCAGGUGACCC |
| DS20-211S | 211 | ggtcacctgacctttctcctgt | 497 | GGUCACCUGACCUUUCUCCUGU | 1355 | AGGAGAAAGGUCAGGUGACC |
| DS20-212S | 212 | gtcacctgacctttctcctgtc | 498 | GUCACCUGACCUUUCUCCUGUC | 1356 | CAGGAGAAAGGUCAGGUGAC |
| DS20-213S | 213 | tcacctgacctttctcctgtcc | 499 | UCACCUGACCUUUCUCCUGUCC | 1357 | ACAGGAGAAAGGUCAGGUGA |
| DS20-214S | 214 | cacctgacctttctcctgtocc | 500 | CACCUGACCUUUCUCCUGUCCC | 1358 | GACAGGAGAAAGGUCAGGUG |
| DS20-215S | 215 | ccccacttgagctcagtggctg | 501 | CCCCACUUGAGCUCAGUGGCUG | 1359 | GCCACUGAGCUCAAGUGGGG |
| DS20-216S | 216 | cccacttgagctcagtggctgg | 502 | CCCACUUGAGCUCAGUGGCUGG | 1360 | AGCCACUGAGCUCAAGUGGG |
| DS20-217S | 217 | ccacttgagctcagtggctggg | 503 | CCACUUGAGCUCAGUGGCUGGG | 1361 | CAGCCACUGAGCUCAAGUGG |
| DS20-218S | 218 | cacttgagctcagtggctgggc | 504 | CACUUGAGCUCAGUGGCUGGGC | 1362 | CCAGCCACUGAGCUCAAGUG |
| DS20-219S | 219 | acttgagctcagtggctgggca | 505 | ACUUGAGCUCAGUGGCUGGGCA | 1363 | CCCAGCCACUGAGCUCAAGU |
| DS20-220S | 220 | cttgagctcagtggctgggcag | 506 | CUUGAGCUCAGUGGCUGGGCAG | 1364 | GCCCAGCCACUGAGCUCAAG |
| DS20-221S | 221 | ttgagctcagtggctgggcagc | 507 | UUGAGCUCAGUGGCUGGGCAGC | 1365 | UGCCCAGCCACUGAGCUCAA |
| DS20-222S | 222 | tgagctcagtggctgggcagca | 508 | UGAGCUCAGUGGCUGGGCAGCA | 1366 | CUGCCCAGCCACUGAGCUCA |
| DS20-223S | 223 | gagctcagtggctgggcagcag | 509 | GAGCUCAGUGGCUGGGCAGCAG | 1367 | GCUGCCCAGCCACUGAGCUC |
| DS20-224S | 224 | agctcagtggctgggcagcagg | 510 | AGCUCAGUGGCUGGGCAGCAGG | 1368 | UGCUGCCCAGCCACUGAGCU |
| DS20-225S | 225 | tcagtggctgggcagcagggga | 511 | UCAGUGGCUGGGCAGCAGGGGA | 1369 | CCCUGCUGCCCAGCCACUGA |
| DS20-226S | 226 | cagtggctgggcagcaggggat | 512 | CAGUGGCUGGGCAGCAGGGGAU | 1370 | CCCCUGCUGCCCAGCCACUG |
| DS20-227S | 227 | agtggctgggcagcaggggatg | 513 | AGUGGCUGGGCAGCAGGGGAUG | 1371 | UCCCCUGCUGCCCAGCCACU |
| DS20-228S | 228 | tggctgggcagcaggggatgca | 514 | UGGCUGGGCAGCAGGGGAUGCA | 1372 | CAUCCCCUGCUGCCCAGCCA |
| DS20-229S | 229 | ggctgggcagcaggggatgcat | 515 | GGCUGGGCAGCAGGGGAUGCAU | 1373 | GCAUCCCCUGCUGCCCAGCC |
| DS20-230S | 230 | gctgggcagcaggggatgcatg | 516 | GCUGGGCAGCAGGGGAUGCAUG | 1374 | UGCAUCCCCUGCUGCCCAGC |
| DS20-231S | 231 | ctgggcagcaggggatgcatgg | 517 | CUGGGCAGCAGGGGAUGCAUGG | 1375 | AUGCAUCCCCUGCUGCCCAG |
| DS20-232S | 232 | tgggcagcaggggatgcatggc | 518 | UGGGCAGCAGGGGAUGCAUGGC | 1376 | CAUGCAUCCCCUGCUGCCCA |
| DS20-233S | 233 | ggcagcaggggatgcatggcca | 519 | GGCAGCAGGGGAUGCAUGGCCA | 1377 | GCCAUGCAUCCCCUGCUGCC |
| DS20-234S | 234 | gcagcaggggatgcatggccac | 520 | GCAGCAGGGGAUGCAUGGCCAC | 1378 | GGCCAUGCAUCCCCUGCUGC |
| DS20-235S | 235 | cagcaggggatgcatggccact | 521 | CAGCAGGGGAUGCAUGGCCACU | 1379 | UGGCCAUGCAUCCCCUGCUG |
| DS20-236S | 236 | agcaggggatgcatggccactg | 522 | AGCAGGGGAUGCAUGGCCACUG | 1380 | GUGGCCAUGCAUCCCCUGCU |
| DS20-237S | 237 | gcaggggatgcatggccactgg | 523 | GCAGGGGAUGCAUGGCCACUGG | 1381 | AGUGGCCAUGCAUCCCCUGC |
| DS20-238S | 238 | caggggatgcatggccactggc | 524 | CAGGGGAUGCAUGGCCACUGGC | 1382 | CAGUGGCCAUGCAUCCCCUG |
| DS20-239S | 239 | aggggatgcatggccactggcc | 525 | AGGGGAUGCAUGGCCACUGGCC | 1383 | CCAGUGGCCAUGCAUCCCCU |
| DS20-240S | 240 | atgcatggccactggccggcca | 526 | AUGCAUGGCCACUGGCCGGCCA | 1384 | GCCGGCCAGUGGCCAUGCAU |
| DS20-241S | 241 | cggccaggtgcagctctcagct | 527 | CGGCCAGGUGCAGCUCUCAGCU | 1385 | CUGAGAGCUGCACCUGGCCG |
| DS20-242S | 242 | ggccaggtgcagctctcagctg | 528 | GGCCAGGUGCAGCUCUCAGCUG | 1386 | GCUGAGAGCUGCACCUGGCC |
| DS20-243S | 243 | gccaggtgcagctctcagctgg | 529 | GCCAGGUGCAGCUCUCAGCUGG | 1387 | AGCUGAGAGCUGCACCUGGC |
| DS20-244S | 244 | ccaggtgcagctctcagctggg | 530 | CCAGGUGCAGCUCUCAGCUGGG | 1388 | CAGCUGAGAGCUGCACCUGG |
| DS20-245S | 245 | caggtgcagctctcagctgggg | 531 | CAGGUGCAGCUCUCAGCUGGGG | 1389 | CCAGCUGAGAGCUGCACCUG |
| DS20-246S | 246 | aggtgcagctctcagctggggt | 532 | AGGUGCAGCUCUCAGCUGGGGU | 1390 | CCCAGCUGAGAGCUGCACCU |
| DS20-247S | 247 | ggtgcagctctcagctggggtg | 533 | GGUGCAGCUCUCAGCUGGGGUG | 1391 | CCCCAGCUGAGAGCUGCACC |
| DS20-248S | 248 | gtgcagctctcagctggggtgt | 534 | GUGCAGCUCUCAGCUGGGGUGU | 1392 | ACCCCAGCUGAGAGCUGCAC |
| DS20-249S | 249 | tgcagctctcagctggggtgtt | 535 | UGCAGCUCUCAGCUGGGGUGUU | 1393 | CACCCCAGCUGAGAGCUGCA |
| DS20-250S | 250 | gcagctctcagctggggtgttc | 536 | GCAGCUCUCAGCUGGGGUGUUC | 1394 | ACACCCCAGCUGAGAGCUGC |
| DS20-251S | 251 | cagctctcagctggggtgttca | 537 | CAGCUCUCAGCUGGGGUGUUCA | 1395 | AACACCCCAGCUGAGAGCUG |
| DS20-252S | 252 | agctctcagctggggtgttcag | 538 | AGCUCUCAGCUGGGGUGUUCAG | 1396 | GAACACCCCAGCUGAGAGCU |
| DS20-253S | 253 | gctctcagctggggtgttcaga | 539 | GCUCUCAGCUGGGGUGUUCAGA | 1397 | UGAACACCCCAGCUGAGAGC |
| DS20-254S | 254 | ctctcagctggggtgttcagag | 540 | CUCUCAGCUGGGGUGUUCAGAG | 1398 | CUGAACACCCCAGCUGAGAG |
| DS20-255S | 255 | tctcagctggggtgttcagagg | 541 | UCUCAGCUGGGGUGUUCAGAGG | 1399 | UCUGAACACCCCAGCUGAGA |
| DS20-256S | 256 | ctcagctggggtgttcagagga | 542 | CUCAGCUGGGGUGUUCAGAGGA | 1400 | CUCUGAACACCCCAGCUGAG |
| DS20-257S | 257 | tcagctggggtgttcagaggac | 543 | UCAGCUGGGGUGUUCAGAGGAC | 1401 | CCUCUGAACACCCCAGCUGA |
| DS20-258S | 258 | cagctggggtgttcagaggacg | 544 | CAGCUGGGGUGUUCAGAGGACG | 1402 | UCCUCUGAACACCCCAGCUG |
| DS20-259S | 259 | agctggggtgttcagaggacgc | 545 | AGCUGGGGUGUUCAGAGGACGC | 1403 | GUCCUCUGAACACCCCAGCU |
| DS20-260S | 260 | gctggggtgttcagaggacgcc | 546 | GCUGGGGUGUUCAGAGGACGCC | 1404 | CGUCCUCUGAACACCCCAGC |
| DS20-261S | 261 | ctggggtgttcagaggacgcct | 547 | CUGGGGUGUUCAGAGGACGCCU | 1405 | GCGUCCUCUGAACACCCCAG |
| DS20-262S | 262 | tggggtgttcagaggacgcctg | 548 | UGGGGUGUUCAGAGGACGCCUG | 1406 | GGCGUCCUCUGAACACCCCA |
| DS20-263S | 263 | ggggtgttcagaggacgcctgt | 549 | GGGGUGUUCAGAGGACGCCUGU | 1407 | AGGCGUCCUCUGAACACCCC |
| DS20-264S | 264 | gggtgttcagaggacgcctgtg | 550 | GGGUGUUCAGAGGACGCCUGUG | 1408 | CAGGCGUCCUCUGAACACCC |
| DS20-265S | 265 | ggtgttcagaggacgcctgtgt | 551 | GGUGUUCAGAGGACGCCUGUGU | 1409 | ACAGGCGUCCUCUGAACACC |
| DS20-266S | 266 | gtgttcagaggacgcctgtgtc | 552 | GUGUUCAGAGGACGCCUGUGUC | 1410 | CACAGGCGUCCUCUGAACAC |
| DS20-267S | 267 | tgttcagaggacgcctgtgtcc | 553 | UGUUCAGAGGACGCCUGUGUCC | 1411 | ACACAGGCGUCCUCUGAACA |
| DS20-268S | 268 | gttcagaggacgcctgtgtcct | 554 | GUUCAGAGGACGCCUGUGUCCU | 1412 | GACACAGGCGUCCUCUGAAC |
| DS20-269S | 269 | ttcagaggacgcctgtgtcctc | 555 | UUCAGAGGACGCCUGUGUCCUC | 1413 | GGACACAGGCGUCCUCUGAA |
| DS20-270S | 270 | tcagaggacgcctgtgtcctcc | 556 | UCAGAGGACGCCUGUGUCCUCC | 1414 | AGGACACAGGCGUCCUCUGA |
| DS20-271S | 271 | cagaggacgcctgtgtcctccc | 557 | CAGAGGACGCCUGUGUCCUCCC | 1415 | GAGGACACAGGCGUCCUCUG |
| DS20-272S | 272 | agaggacgcctgtgtcctcccc | 558 | AGAGGACGCCUGUGUCCUCCCC | 1416 | GGAGGACACAGGCGUCCUCU |
| DS20-273S | 273 | gaggacgcctgtgtcctcccct | 559 | GAGGACGCCUGUGUCCUCCCCU | 1417 | GGGAGGACACAGGCGUCCUC |
| DS20-274S | 274 | aggacgcctgtgtcctcccctc | 560 | AGGACGCCUGUGUCCUCCCCUC | 1418 | GGGGAGGACACAGGCGUCCU |
| DS20-275S | 275 | ctctgtcacccttggaggcaga | 561 | CUCUGUCACCCUUGGAGGCAGA | 1419 | UGCCUCCAAGGGUGACAGAG |
| DS20-276S | 276 | tctgtcacccttggaggcagag | 562 | UCUGUCACCCUUGGAGGCAGAG | 1420 | CUGCCUCCAAGGGUGACAGA |
| DS20-277S | 277 | ctgtcacccttggaggcagaga | 563 | CUGUCACCCUUGGAGGCAGAGA | 1421 | UCUGCCUCCAAGGGUGACAG |
| DS20-278S | 278 | tgtcacccttggaggcagagaa | 564 | UGUCACCCUUGGAGGCAGAGAA | 1422 | CUCUGCCUCCAAGGGUGACA |
| DS20-279S | 279 | gtcacccttggaggcagagaac | 565 | GUCACCCUUGGAGGCAGAGAAC | 1423 | UCUCUGCCUCCAAGGGUGAC |
| DS20-280S | 280 | tcacccttggaggcagagaact | 566 | UCACCCUUGGAGGCAGAGAACU | 1424 | UUCUCUGCCUCCAAGGGUGA |
| DS20-281S | 281 | cacccttggaggcagagaactt | 567 | CACCCUUGGAGGCAGAGAACUU | 1425 | GUUCUCUGCCUCCAAGGGUG |
| DS20-282S | 282 | acccttggaggcagagaacttt | 568 | ACCCUUGGAGGCAGAGAACUUU | 1426 | AGUUCUCUGCCUCCAAGGGU |
| DS20-283S | 283 | cccttggaggcagagaactttg | 569 | CCCUUGGAGGCAGAGAACUUUG | 1427 | AAGUUCUCUGCCUCCAAGGG |
| DS20-284S | 284 | ccttggaggcagagaactttgc | 570 | CCUUGGAGGCAGAGAACUUUGC | 1428 | AAAGUUCUCUGCCUCCAAGG |
| DS20-285S | 285 | cttggaggcagagaactttgcc | 571 | CUUGGAGGCAGAGAACUUUGCC | 1429 | CAAAGUUCUCUGCCUCCAAG |
| DS20-286S | 286 | ttggaggcagagaactttgccc | 572 | UUGGAGGCAGAGAACUUUGCCC | 1430 | GCAAAGUUCUCUGCCUCCAA |
| DS20-001A | 1 | cgtaagatgtggaccgctggag | 573 | UAAGAUGUGGACCGCUGGAG | 859 | CUCCAGCGGUCCACAUCUUACG |
| DS20-002A | 2 | gtaagatgtggaccgctggaga | 574 | AAGAUGUGGACCGCUGGAGA | 860 | UCUCCAGCGGUCCACAUCUUAC |
| DS20-003A | 3 | taagatgtggaccgctggagaa | 575 | AGAUGUGGACCGCUGGAGAA | 861 | UUCUCCAGCGGUCCACAUCUUA |
| DS20-004A | 4 | aagatgtggaccgctggagaat | 576 | GAUGUGGACCGCUGGAGAAU | 862 | AUUCUCCAGCGGUCCACAUCUU |
| DS20-005A | 5 | agatgtggaccgctggagaatg | 577 | AUGUGGACCGCUGGAGAAUG | 863 | CAUUCUCCAGCGGUCCACAUCU |
| DS20-006A | 6 | gatgtggaccgctggagaatgg | 578 | UGUGGACCGCUGGAGAAUGG | 864 | CCAUUCUCCAGCGGUCCACAUC |
| DS20-007A | 7 | atgtggaccgctggagaatggg | 579 | GUGGACCGCUGGAGAAUGGG | 865 | CCCAUUCUCCAGCGGUCCACAU |
| DS20-008A | 8 | tgtggaccgctggagaatgggg | 580 | UGGACCGCUGGAGAAUGGGG | 866 | CCCCAUUCUCCAGCGGUCCACA |
| DS20-009A | 9 | ggggtgctgcctgcagtcaaaa | 581 | GGUGCUGCCUGCAGUCAAAA | 867 | UUUUGACUGCAGGCAGCACCCC |
| DS20-010A | 10 | gggtgctgcctgcagtcaaaac | 582 | GUGCUGCCUGCAGUCAAAAC | 868 | GUUUUGACUGCAGGCAGCACCC |
| DS20-011A | 11 | ggtgctgcctgcagtcaaaacg | 583 | UGCUGCCUGCAGUCAAAACG | 869 | CGUUUUGACUGCAGGCAGCACC |
| DS20-012A | 12 | gtgctgcctgcagtcaaaacgg | 584 | GCUGCCUGCAGUCAAAACGG | 870 | CCGUUUUGACUGCAGGCAGCAC |
| DS20-013A | 13 | tgctgcctgcagtcaaaacgga | 585 | CUGCCUGCAGUCAAAACGGA | 871 | UCCGUUUUGACUGCAGGCAGCA |
| DS20-014A | 14 | gctgcctgcagtcaaaacggag | 586 | UGCCUGCAGUCAAAACGGAG | 872 | CUCCGUUUUGACUGCAGGCAGC |
| DS20-015A | 15 | ctgcctgcagtcaaaacggagt | 587 | GCCUGCAGUCAAAACGGAGU | 873 | ACUCCGUUUUGACUGCAGGCAG |
| DS20-016A | 16 | tgcctgcagtcaaaacggagtg | 588 | CCUGCAGUCAAAACGGAGUG | 874 | CACUCCGUUUUGACUGCAGGCA |
| DS20-017A | 17 | gcctgcagtcaaaacggagtgg | 589 | CUGCAGUCAAAACGGAGUGG | 875 | CCACUCCGUUUUGACUGCAGGC |
| DS20-018A | 18 | cctgcagtcaaaacggagtggg | 590 | UGCAGUCAAAACGGAGUGGG | 876 | CCCACUCCGUUUUGACUGCAGG |
| DS20-019A | 19 | ctgcagtcaaaacggagtgggg | 591 | GCAGUCAAAACGGAGUGGGG | 877 | CCCCACUCCGUUUUGACUGCAG |
| DS20-020A | 20 | ggtgcccagctcagggccagaa | 592 | UGCCCAGCUCAGGGCCAGAA | 878 | UUCUGGCCCUGAGCUGGGCACC |
| DS20-021A | 21 | gtgcccagctcagggccagaat | 593 | GCCCAGCUCAGGGCCAGAAU | 879 | AUUCUGGCCCUGAGCUGGGCAC |
| DS20-022A | 22 | tgcccagctcagggccagaatg | 594 | CCCAGCUCAGGGCCAGAAUG | 880 | CAUUCUGGCCCUGAGCUGGGCA |
| DS20-023A | 23 | gcccagctcagggccagaatga | 595 | CCAGCUCAGGGCCAGAAUGA | 881 | UCAUUCUGGCCCUGAGCUGGGC |
| DS20-024A | 24 | cccagctcagggccagaatgat | 596 | CAGCUCAGGGCCAGAAUGAU | 882 | AUCAUUCUGGCCCUGAGCUGGG |
| DS20-025A | 25 | ccagctcagggccagaatgatc | 597 | AGCUCAGGGCCAGAAUGAUC | 883 | GAUCAUUCUGGCCCUGAGCUGG |
| DS20-026A | 26 | cagctcagggccagaatgatcc | 598 | GCUCAGGGCCAGAAUGAUCC | 884 | GGAUCAUUCUGGCCCUGAGCUG |
| DS20-027A | 27 | agctcagggccagaatgatcct | 599 | CUCAGGGCCAGAAUGAUCCU | 885 | AGGAUCAUUCUGGCCCUGAGCU |
| DS20-028A | 28 | gctcagggccagaatgatccta | 600 | UCAGGGCCAGAAUGAUCCUA | 886 | UAGGAUCAUUCUGGCCCUGAGC |
| DS20-029A | 29 | ctcagggccagaatgatcctat | 601 | CAGGGCCAGAAUGAUCCUAU | 887 | AUAGGAUCAUUCUGGCCCUGAG |
| DS20-030A | 30 | tcagggccagaatgatcctatt | 602 | AGGGCCAGAAUGAUCCUAUU | 888 | AAUAGGAUCAUUCUGGCCCUGA |
| DS20-031A | 31 | cagggccagaatgatcctattc | 603 | GGGCCAGAAUGAUCCUAUUC | 889 | GAAUAGGAUCAUUCUGGCCCUG |
| DS20-032A | 32 | agggccagaatgatcctattcc | 604 | GGCCAGAAUGAUCCUAUUCC | 890 | GGAAUAGGAUCAUUCUGGCCCU |
| DS20-033A | 33 | gggccagaatgatcctattccc | 605 | GCCAGAAUGAUCCUAUUCCC | 891 | GGGAAUAGGAUCAUUCUGGCCC |
| DS20-034A | 34 | ggccagaatgatcctattcccg | 606 | CCAGAAUGAUCCUAUUCCCG | 892 | CGGGAAUAGGAUCAUUCUGGCC |
| DS20-035A | 35 | gccagaatgatcctattcccgg | 607 | CAGAAUGAUCCUAUUCCCGG | 893 | CCGGGAAUAGGAUCAUUCUGGC |
| DS20-036A | 36 | ccagaatgatcctattcccggc | 608 | AGAAUGAUCCUAUUCCCGGC | 894 | GCCGGGAAUAGGAUCAUUCUGG |
| DS20-037A | 37 | cagaatgatcctattcccggca | 609 | GAAUGAUCCUAUUCCCGGCA | 895 | UGCCGGGAAUAGGAUCAUUCUG |
| DS20-038A | 38 | agaatgatcctattcccggcac | 610 | AAUGAUCCUAUUCCCGGCAC | 896 | GUGCCGGGAAUAGGAUCAUUCU |
| DS20-039A | 39 | gaatgatcctattcccggcact | 611 | AUGAUCCUAUUCCCGGCACU | 897 | AGUGCCGGGAAUAGGAUCAUUC |
| DS20-040A | 40 | aatgatcctattcccggcactt | 612 | UGAUCCUAUUCCCGGCACUU | 898 | AAGUGCCGGGAAUAGGAUCAUU |
| DS20-041A | 41 | atgatcctattcccggcacttc | 613 | GAUCCUAUUCCCGGCACUUC | 899 | GAAGUGCCGGGAAUAGGAUCAU |
| DS20-042A | 42 | tgatcctattcccggcacttct | 614 | AUCCUAUUCCCGGCACUUCU | 900 | AGAAGUGCCGGGAAUAGGAUCA |
| DS20-043A | 43 | gatcctattcccggcacttctc | 615 | UCCUAUUCCCGGCACUUCUC | 901 | GAGAAGUGCCGGGAAUAGGAUC |
| DS20-044A | 44 | atcctattcccggcacttctca | 616 | CCUAUUCCCGGCACUUCUCA | 902 | UGAGAAGUGCCGGGAAUAGGAU |
| DS20-045A | 45 | tcctattcccggcacttctcag | 617 | CUAUUCCCGGCACUUCUCAG | 903 | CUGAGAAGUGCCGGGAAUAGGA |
| DS20-046A | 46 | cctattcccggcacttctcagt | 618 | UAUUCCCGGCACUUCUCAGU | 904 | ACUGAGAAGUGCCGGGAAUAGG |
| DS20-047A | 47 | ctattcccggcacttctcagtg | 619 | AUUCCCGGCACUUCUCAGUG | 905 | CACUGAGAAGUGCCGGGAAUAG |
| DS20-048A | 48 | tattcccggcacttctcagtga | 620 | UUCCCGGCACUUCUCAGUGA | 906 | UCACUGAGAAGUGCCGGGAAUA |
| DS20-049A | 49 | attcccggcacttctcagtgag | 621 | UCCCGGCACUUCUCAGUGAG | 907 | CUCACUGAGAAGUGCCGGGAAU |
| DS20-050A | 50 | ttcccggcacttctcagtgagg | 622 | CCCGGCACUUCUCAGUGAGG | 908 | CCUCACUGAGAAGUGCCGGGAA |
| DS20-051A | 51 | tcccggcacttctcagtgaggc | 623 | CCGGCACUUCUCAGUGAGGC | 909 | GCCUCACUGAGAAGUGCCGGGA |
| DS20-052A | 52 | cccggcacttctcagtgaggct | 624 | CGGCACUUCUCAGUGAGGCU | 910 | AGCCUCACUGAGAAGUGCCGGG |
| DS20-053A | 53 | ccggcacttctcagtgaggctc | 625 | GGCACUUCUCAGUGAGGCUC | 911 | GAGCCUCACUGAGAAGUGCCGG |
| DS20-054A | 54 | cggcacttctcagtgaggctct | 626 | GCACUUCUCAGUGAGGCUCU | 912 | AGAGCCUCACUGAGAAGUGCCG |
| DS20-055A | 55 | ggcacttctcagtgaggctctg | 627 | CACUUCUCAGUGAGGCUCUG | 913 | CAGAGCCUCACUGAGAAGUGCC |
| DS20-056A | 56 | gcacttctcagtgaggctctgt | 628 | ACUUCUCAGUGAGGCUCUGU | 914 | ACAGAGCCUCACUGAGAAGUGC |
| DS20-057A | 57 | cacttctcagtgaggctctgtg | 629 | CUUCUCAGUGAGGCUCUGUG | 915 | CACAGAGCCUCACUGAGAAGUG |
| DS20-058A | 58 | acttctcagtgaggctctgtgg | 630 | UUCUCAGUGAGGCUCUGUGG | 916 | CCACAGAGCCUCACUGAGAAGU |
| DS20-059A | 59 | cttctcagtgaggctctgtggc | 631 | UCUCAGUGAGGCUCUGUGGC | 917 | GCCACAGAGCCUCACUGAGAAG |
| DS20-060A | 60 | ttctcagtgaggctctgtggct | 632 | CUCAGUGAGGCUCUGUGGCU | 918 | AGCCACAGAGCCUCACUGAGAA |
| DS20-061A | 61 | tctcagtgaggctctgtggctc | 633 | UCAGUGAGGCUCUGUGGCUC | 919 | GAGCCACAGAGCCUCACUGAGA |
| DS20-062A | 62 | ctcagtgaggctctgtggctca | 634 | CAGUGAGGCUCUGUGGCUCA | 920 | UGAGCCACAGAGCCUCACUGAG |
| DS20-063A | 63 | tcagtgaggctctgtggctcac | 635 | AGUGAGGCUCUGUGGCUCAC | 921 | GUGAGCCACAGAGCCUCACUGA |
| DS20-064A | 64 | cagtgaggctctgtggctcacc | 636 | GUGAGGCUCUGUGGCUCACC | 922 | GGUGAGCCACAGAGCCUCACUG |
| DS20-065A | 65 | agtgaggctctgtggctcacct | 637 | UGAGGCUCUGUGGCUCACCU | 923 | AGGUGAGCCACAGAGCCUCACU |
| DS20-066A | 66 | gtgaggctctgtggctcaccta | 638 | GAGGCUCUGUGGCUCACCUA | 924 | UAGGUGAGCCACAGAGCCUCAC |
| DS20-067A | 67 | tgaggctctgtggctcacctaa | 639 | AGGCUCUGUGGCUCACCUAA | 925 | UUAGGUGAGCCACAGAGCCUCA |
| DS20-068A | 68 | gaggctctgtggctcacctaag | 640 | GGCUCUGUGGCUCACCUAAG | 926 | CUUAGGUGAGCCACAGAGCCUC |
| DS20-069A | 69 | aggctctgtggctcacctaaga | 641 | GCUCUGUGGCUCACCUAAGA | 927 | UCUUAGGUGAGCCACAGAGCCU |
| DS20-070A | 70 | ggctctgtggctcacctaagaa | 642 | CUCUGUGGCUCACCUAAGAA | 928 | UUCUUAGGUGAGCCACAGAGCC |
| DS20-071A | 71 | gctctgtggctcacctaagaaa | 643 | UCUGUGGCUCACCUAAGAAA | 929 | UUUCUUAGGUGAGCCACAGAGC |
| DS20-072A | 72 | ctctgtggctcacctaagaaac | 644 | CUGUGGCUCACCUAAGAAAC | 930 | GUUUCUUAGGUGAGCCACAGAG |
| DS20-073A | 73 | tctgtggctcacctaagaaacc | 645 | UGUGGCUCACCUAAGAAACC | 931 | GGUUUCUUAGGUGAGCCACAGA |
| DS20-074A | 74 | ctgtggctcacctaagaaacca | 646 | GUGGCUCACCUAAGAAACCA | 932 | UGGUUUCUUAGGUGAGCCACAG |
| DS20-075A | 75 | tgtggctcacctaagaaaccag | 647 | UGGCUCACCUAAGAAACCAG | 933 | CUGGUUUCUUAGGUGAGCCACA |
| DS20-076A | 76 | gtggctcacctaagaaaccagc | 648 | GGCUCACCUAAGAAACCAGC | 934 | GCUGGUUUCUUAGGUGAGCCAC |
| DS20-077A | 77 | tggctcacctaagaaaccagcc | 649 | GCUCACCUAAGAAACCAGCC | 935 | GGCUGGUUUCUUAGGUGAGCCA |
| DS20-078A | 78 | ggctcacctaagaaaccagcct | 650 | CUCACCUAAGAAACCAGCCU | 936 | AGGCUGGUUUCUUAGGUGAGCC |
| DS20-079A | 79 | gctcacctaagaaaccagcctc | 651 | UCACCUAAGAAACCAGCCUC | 937 | GAGGCUGGUUUCUUAGGUGAGC |
| DS20-080A | 80 | ctcacctaagaaaccagcctcc | 652 | CACCUAAGAAACCAGCCUCC | 938 | GGAGGCUGGUUUCUUAGGUGAG |
| DS20-081A | 81 | tcacctaagaaaccagcctccc | 653 | ACCUAAGAAACCAGCCUCCC | 939 | GGGAGGCUGGUUUCUUAGGUGA |
| DS20-082A | 82 | cacctaagaaaccagcctccct | 654 | CCUAAGAAACCAGCCUCCCU | 940 | AGGGAGGCUGGUUUCUUAGGUG |
| DS20-083A | 83 | acctaagaaaccagcctccctt | 655 | CUAAGAAACCAGCCUCCCUU | 941 | AAGGGAGGCUGGUUUCUUAGGU |
| DS20-084A | 84 | cctaagaaaccagcctcccttg | 656 | UAAGAAACCAGCCUCCCUUG | 942 | CAAGGGAGGCUGGUUUCUUAGG |
| DS20-085A | 85 | ctaagaaaccagcctcccttgc | 657 | AAGAAACCAGCCUCCCUUGC | 943 | GCAAGGGAGGCUGGUUUCUUAG |
| DS20-086A | 86 | taagaaaccagcctcccttgca | 658 | AGAAACCAGCCUCCCUUGCA | 944 | UGCAAGGGAGGCUGGUUUCUUA |
| DS20-087A | 87 | aagaaaccagcctcccttgcag | 659 | GAAACCAGCCUCCCUUGCAG | 945 | CUGCAAGGGAGGCUGGUUUCUU |
| DS20-088A | 88 | agaaaccagcctcccttgcagg | 660 | AAACCAGCCUCCCUUGCAGG | 946 | CCUGCAAGGGAGGCUGGUUUCU |
| DS20-089A | 89 | gaaaccagcctcccttgcaggc | 661 | AACCAGCCUCCCUUGCAGGC | 947 | GCCUGCAAGGGAGGCUGGUUUC |
| DS20-090A | 90 | aaaccagcctcccttgcaggca | 662 | ACCAGCCUCCCUUGCAGGCA | 948 | UGCCUGCAAGGGAGGCUGGUUU |
| DS20-091A | 91 | aaccagcctcccttgcaggcaa | 663 | CCAGCCUCCCUUGCAGGCAA | 949 | UUGCCUGCAAGGGAGGCUGGUU |
| DS20-092A | 92 | accagcctcccttgcaggcaac | 664 | CAGCCUCCCUUGCAGGCAAC | 950 | GUUGCCUGCAAGGGAGGCUGGU |
| DS20-093A | 93 | ccagcctcccttgcaggcaacg | 665 | AGCCUCCCUUGCAGGCAACG | 951 | CGUUGCCUGCAAGGGAGGCUGG |
| DS20-094A | 94 | cagcctcccttgcaggcaacgg | 666 | GCCUCCCUUGCAGGCAACGG | 952 | CCGUUGCCUGCAAGGGAGGCUG |
| DS20-095A | 95 | agcctcccttgcaggcaacggc | 667 | CCUCCCUUGCAGGCAACGGC | 953 | GCCGUUGCCUGCAAGGGAGGCU |
| DS20-096A | 96 | cctcccttgcaggcaacggcct | 668 | UCCCUUGCAGGCAACGGCCU | 954 | AGGCCGUUGCCUGCAAGGGAGG |
| DS20-097A | 97 | ctcccttgcaggcaacggccta | 669 | CCCUUGCAGGCAACGGCCUA | 955 | UAGGCCGUUGCCUGCAAGGGAG |
| DS20-098A | 98 | tcccttgcaggcaacggcctag | 670 | CCUUGCAGGCAACGGCCUAG | 956 | CUAGGCCGUUGCCUGCAAGGGA |
| DS20-099A | 99 | cccttgcaggcaacggcctagc | 671 | CUUGCAGGCAACGGCCUAGC | 957 | GCUAGGCCGUUGCCUGCAAGGG |
| DS20-100A | 100 | ccttgcaggcaacggcctagct | 672 | UUGCAGGCAACGGCCUAGCU | 958 | AGCUAGGCCGUUGCCUGCAAGG |
| DS20-101A | 101 | cttgcaggcaacggcctagctg | 673 | UGCAGGCAACGGCCUAGCUG | 959 | CAGCUAGGCCGUUGCCUGCAAG |
| DS20-102A | 102 | ttgcaggcaacggcctagctgg | 674 | GCAGGCAACGGCCUAGCUGG | 960 | CCAGCUAGGCCGUUGCCUGCAA |
| DS20-103A | 103 | tgcaggcaacggcctagctggc | 675 | CAGGCAACGGCCUAGCUGGC | 961 | GCCAGCUAGGCCGUUGCCUGCA |
| DS20-104A | 104 | caggcaacggcctagctggcct | 676 | GGCAACGGCCUAGCUGGCCU | 962 | AGGCCAGCUAGGCCGUUGCCUG |
| DS20-105A | 105 | aggcaacggcctagctggcctg | 677 | GCAACGGCCUAGCUGGCCUG | 963 | CAGGCCAGCUAGGCCGUUGCCU |
| DS20-106A | 106 | gcaacggcctagctggcctggt | 678 | AACGGCCUAGCUGGCCUGGU | 964 | ACCAGGCCAGCUAGGCCGUUGC |
| DS20-107A | 107 | caacggcctagctggcctggtc | 679 | ACGGCCUAGCUGGCCUGGUC | 965 | GACCAGGCCAGCUAGGCCGUUG |
| DS20-108A | 108 | aacggcctagctggcctggtct | 680 | CGGCCUAGCUGGCCUGGUCU | 966 | AGACCAGGCCAGCUAGGCCGUU |
| DS20-109A | 109 | acggcctagctggcctggtctg | 681 | GGCCUAGCUGGCCUGGUCUG | 967 | CAGACCAGGCCAGCUAGGCCGU |
| DS20-110A | 110 | ggcctagctggcctggtctgga | 682 | CCUAGCUGGCCUGGUCUGGA | 968 | UCCAGACCAGGCCAGCUAGGCC |
| DS20-111A | 111 | gcctagctggcctggtctggag | 683 | CUAGCUGGCCUGGUCUGGAG | 969 | CUCCAGACCAGGCCAGCUAGGC |
| DS20-112A | 112 | cctagctggcctggtctggagg | 684 | UAGCUGGCCUGGUCUGGAGG | 970 | CCUCCAGACCAGGCCAGCUAGG |
| DS20-113A | 113 | ctagctggcctggtctggaggc | 685 | AGCUGGCCUGGUCUGGAGGC | 971 | GCCUCCAGACCAGGCCAGCUAG |
| DS20-114A | 114 | tagctggcctggtctggaggct | 686 | GCUGGCCUGGUCUGGAGGCU | 972 | AGCCUCCAGACCAGGCCAGCUA |
| DS20-115A | 115 | agctggcctggtctggaggctc | 687 | CUGGCCUGGUCUGGAGGCUC | 973 | GAGCCUCCAGACCAGGCCAGCU |
| DS20-116A | 116 | gctggcctggtctggaggctct | 688 | UGGCCUGGUCUGGAGGCUCU | 974 | AGAGCCUCCAGACCAGGCCAGC |
| DS20-117A | 117 | ctggcctggtctggaggctctc | 689 | GGCCUGGUCUGGAGGCUCUC | 975 | GAGAGCCUCCAGACCAGGCCAG |
| DS20-118A | 118 | tttacatccacacccaagatac | 690 | UACAUCCACACCCAAGAUAC | 976 | GUAUCUUGGGUGUGGAUGUAAA |
| DS20-119A | 119 | tcttgagatttgactcgcatga | 691 | UUGAGAUUUGACUCGCAUGA | 977 | UCAUGCGAGUCAAAUCUCAAGA |
| DS20-120A | 120 | cttgagatttgactcgcatgat | 692 | UGAGAUUUGACUCGCAUGAU | 978 | AUCAUGCGAGUCAAAUCUCAAG |
| DS20-121A | 121 | tgagatttgactcgcatgattg | 693 | AGAUUUGACUCGCAUGAUUG | 979 | CAAUCAUGCGAGUCAAAUCUCA |
| DS20-122A | 122 | gagatttgactcgcatgattgc | 694 | GAUUUGACUCGCAUGAUUGC | 980 | GCAAUCAUGCGAGUCAAAUCUC |
| DS20-123A | 123 | agatttgactcgcatgattgct | 695 | AUUUGACUCGCAUGAUUGCU | 981 | AGCAAUCAUGCGAGUCAAAUCU |
| DS20-124A | 124 | gatttgactcgcatgattgcta | 696 | UUUGACUCGCAUGAUUGCUA | 982 | UAGCAAUCAUGCGAGUCAAAUC |
| DS20-125A | 125 | tttgactcgcatgattgctatg | 697 | UGACUCGCAUGAUUGCUAUG | 983 | CAUAGCAAUCAUGCGAGUCAAA |
| DS20-126A | 126 | ttgactcgcatgattgctatgg | 698 | GACUCGCAUGAUUGCUAUGG | 984 | CCAUAGCAAUCAUGCGAGUCAA |
| DS20-127A | 127 | tgactcgcatgattgctatggg | 699 | ACUCGCAUGAUUGCUAUGGG | 985 | CCCAUAGCAAUCAUGCGAGUCA |
| DS20-128A | 128 | gactcgcatgattgctatggga | 700 | CUCGCAUGAUUGCUAUGGGA | 986 | UCCCAUAGCAAUCAUGCGAGUC |
| DS20-129A | 129 | actcgcatgattgctatgggac | 701 | UCGCAUGAUUGCUAUGGGAC | 987 | GUCCCAUAGCAAUCAUGCGAGU |
| DS20-130A | 130 | ctcgcatgattgctatgggaca | 702 | CGCAUGAUUGCUAUGGGACA | 988 | UGUCCCAUAGCAAUCAUGCGAG |
| DS20-131A | 131 | tcgcatgattgctatgggacaa | 703 | GCAUGAUUGCUAUGGGACAA | 989 | UUGUCCCAUAGCAAUCAUGCGA |
| DS20-132A | 132 | cgcatgattgctatgggacaag | 704 | CAUGAUUGCUAUGGGACAAG | 990 | CUUGUCCCAUAGCAAUCAUGCG |
| DS20-133A | 133 | gcatgattgctatgggacaagt | 705 | AUGAUUGCUAUGGGACAAGU | 991 | ACUUGUCCCAUAGCAAUCAUGC |
| DS20-134A | 134 | catgattgctatgggacaagtt | 706 | UGAUUGCUAUGGGACAAGUU | 992 | AACUUGUCCCAUAGCAAUCAUG |
| DS20-135A | 135 | gattgctatgggacaagttttc | 707 | UUGCUAUGGGACAAGUUUUC | 993 | GAAAACUUGUCCCAUAGCAAUC |
| DS20-136A | 136 | tgctatgggacaagttttcatc | 708 | CUAUGGGACAAGUUUUCAUC | 994 | GAUGAAAACUUGUCCCAUAGCA |
| DS20-137A | 137 | gctatgggacaagttttcatct | 709 | UAUGGGACAAGUUUUCAUCU | 995 | AGAUGAAAACUUGUCCCAUAGC |
| DS20-138A | 138 | ctatgggacaagttttcatctg | 710 | AUGGGACAAGUUUUCAUCUG | 996 | CAGAUGAAAACUUGUCCCAUAG |
| DS20-139A | 139 | tatgggacaagttttcatctgc | 711 | UGGGACAAGUUUUCAUCUGC | 997 | GCAGAUGAAAACUUGUCCCAUA |
| DS20-140A | 140 | atgggacaagttttcatctgca | 712 | GGGACAAGUUUUCAUCUGCA | 998 | UGCAGAUGAAAACUUGUCCCAU |
| DS20-141A | 141 | tgggacaagttttcatctgcag | 713 | GGACAAGUUUUCAUCUGCAG | 999 | CUGCAGAUGAAAACUUGUCCCA |
| DS20-142A | 142 | gggacaagttttcatctgcagt | 714 | GACAAGUUUUCAUCUGCAGU | 1000 | ACUGCAGAUGAAAACUUGUCCC |
| DS20-143A | 143 | ggacaagttttcatctgcagtt | 715 | ACAAGUUUUCAUCUGCAGUU | 1001 | AACUGCAGAUGAAAACUUGUCC |
| DS20-144A | 144 | ctgcagtttaaatctgtttccc | 716 | GCAGUUUAAAUCUGUUUCCC | 1002 | GGGAAACAGAUUUAAACUGCAG |
| DS20-145A | 145 | taggggtttggaattctagatc | 717 | GGGGUUUGGAAUUCUAGAUC | 1003 | GAUCUAGAAUUCCAAACCCCUA |
| DS20-146A | 146 | aggggtttggaattctagatcg | 718 | GGGUUUGGAAUUCUAGAUCG | 1004 | CGAUCUAGAAUUCCAAACCCCU |
| DS20-147A | 147 | ggggtttggaattctagatcgt | 719 | GGUUUGGAAUUCUAGAUCGU | 1005 | ACGAUCUAGAAUUCCAAACCCC |
| DS20-148A | 148 | gggtttggaattctagatcgta | 720 | GUUUGGAAUUCUAGAUCGUA | 1006 | UACGAUCUAGAAUUCCAAACCC |
| DS20-149A | 149 | gatcgtatttgaagtgttggtg | 721 | UCGUAUUUGAAGUGUUGGUG | 1007 | CACCAACACUUCAAAUACGAUC |
| DS20-150A | 150 | atcgtatttgaagtgttggtgc | 722 | CGUAUUUGAAGUGUUGGUGC | 1008 | GCACCAACACUUCAAAUACGAU |
| DS20-151A | 151 | tcgtatttgaagtgttggtgcc | 723 | GUAUUUGAAGUGUUGGUGCC | 1009 | GGCACCAACACUUCAAAUACGA |
| DS20-152A | 152 | cgtatttgaagtgttggtgcca | 724 | UAUUUGAAGUGUUGGUGCCA | 1010 | UGGCACCAACACUUCAAAUACG |
| DS20-153A | 153 | gtatttgaagtgttggtgccac | 725 | AUUUGAAGUGUUGGUGCCAC | 1011 | GUGGCACCAACACUUCAAAUAC |
| DS20-154A | 154 | tatttgaagtgttggtgccaca | 726 | UUUGAAGUGUUGGUGCCACA | 1012 | UGUGGCACCAACACUUCAAAUA |
| DS20-155A | 155 | atttgaagtgttggtgccacac | 727 | UUGAAGUGUUGGUGCCACAC | 1013 | GUGUGGCACCAACACUUCAAAU |
| DS20-156A | 156 | cacaccttaacacctgcacgct | 728 | CACCUUAACACCUGCACGCU | 1014 | AGCGUGCAGGUGUUAAGGUGUG |
| DS20-157A | 157 | acaccttaacacctgcacgctg | 729 | ACCUUAACACCUGCACGCUG | 1015 | CAGCGUGCAGGUGUUAAGGUGU |
| DS20-158A | 158 | caccttaacacctgcacgctgg | 730 | CCUUAACACCUGCACGCUGG | 1016 | CCAGCGUGCAGGUGUUAAGGUG |
| DS20-159A | 159 | accttaacacctgcacgctggc | 731 | CUUAACACCUGCACGCUGGC | 1017 | GCCAGCGUGCAGGUGUUAAGGU |
| DS20-160A | 160 | ccttaacacctgcacgctggca | 732 | UUAACACCUGCACGCUGGCA | 1018 | UGCCAGCGUGCAGGUGUUAAGG |
| DS20-161A | 161 | cttaacacctgcacgctggcaa | 733 | UAACACCUGCACGCUGGCAA | 1019 | UUGCCAGCGUGCAGGUGUUAAG |
| DS20-162A | 162 | ttaacacctgcacgctggcaac | 734 | AACACCUGCACGCUGGCAAC | 1020 | GUUGCCAGCGUGCAGGUGUUAA |
| DS20-163A | 163 | taacacctgcacgctggcaaca | 735 | ACACCUGCACGCUGGCAACA | 1021 | UGUUGCCAGCGUGCAGGUGUUA |
| DS20-164A | 164 | aacacctgcacgctggcaacaa | 736 | CACCUGCACGCUGGCAACAA | 1022 | UUGUUGCCAGCGUGCAGGUGUU |
| DS20-165A | 165 | acacctgcacgctggcaacaaa | 737 | ACCUGCACGCUGGCAACAAA | 1023 | UUUGUUGCCAGCGUGCAGGUGU |
| DS20-166A | 166 | cacctgcacgctggcaacaaaa | 738 | CCUGCACGCUGGCAACAAAA | 1024 | UUUUGUUGCCAGCGUGCAGGUG |
| DS20-167A | 167 | acctgcacgctggcaacaaaac | 739 | CUGCACGCUGGCAACAAAAC | 1025 | GUUUUGUUGCCAGCGUGCAGGU |
| DS20-168A | 168 | cctgcacgctggcaacaaaacc | 740 | UGCACGCUGGCAACAAAACC | 1026 | GGUUUUGUUGCCAGCGUGCAGG |
| DS20-169A | 169 | ctgcacgctggcaacaaaaccg | 741 | GCACGCUGGCAACAAAACCG | 1027 | CGGUUUUGUUGCCAGCGUGCAG |
| DS20-170A | 170 | tgcacgctggcaacaaaaccgt | 742 | CACGCUGGCAACAAAACCGU | 1028 | ACGGUUUUGUUGCCAGCGUGCA |
| DS20-171A | 171 | gcacgctggcaacaaaaccgtc | 743 | ACGCUGGCAACAAAACCGUC | 1029 | GACGGUUUUGUUGCCAGCGUGC |
| DS20-172A | 172 | cacgctggcaacaaaaccgtcc | 744 | CGCUGGCAACAAAACCGUCC | 1030 | GGACGGUUUUGUUGCCAGCGUG |
| DS20-173A | 173 | acgctggcaacaaaaccgtccg | 745 | GCUGGCAACAAAACCGUCCG | 1031 | CGGACGGUUUUGUUGCCAGCGU |
| DS20-174A | 174 | cgctggcaacaaaaccgtccgc | 746 | CUGGCAACAAAACCGUCCGC | 1032 | GCGGACGGUUUUGUUGCCAGCG |
| DS20-175A | 175 | gctggcaacaaaaccgtccgct | 747 | UGGCAACAAAACCGUCCGCU | 1033 | AGCGGACGGUUUUGUUGCCAGC |
| DS20-176A | 176 | ctggcaacaaaaccgtccgctc | 748 | GGCAACAAAACCGUCCGCUC | 1034 | GAGCGGACGGUUUUGUUGCCAG |
| DS20-177A | 177 | tggcaacaaaaccgtccgctct | 749 | GCAACAAAACCGUCCGCUCU | 1035 | AGAGCGGACGGUUUUGUUGCCA |
| DS20-178A | 178 | ggcaacaaaaccgtccgctctg | 750 | CAACAAAACCGUCCGCUCUG | 1036 | CAGAGCGGACGGUUUUGUUGCC |
| DS20-179A | 179 | gcaacaaaaccgtccgctctgc | 751 | AACAAAACCGUCCGCUCUGC | 1037 | GCAGAGCGGACGGUUUUGUUGC |
| DS20-180A | 180 | caacaaaaccgtccgctctgca | 752 | ACAAAACCGUCCGCUCUGCA | 1038 | UGCAGAGCGGACGGUUUUGUUG |
| DS20-181A | 181 | aacaaaaccgtccgctctgcag | 753 | CAAAACCGUCCGCUCUGCAG | 1039 | CUGCAGAGCGGACGGUUUUGUU |
| DS20-182A | 182 | acaaaaccgtccgctctgcagc | 754 | AAAACCGUCCGCUCUGCAGC | 1040 | GCUGCAGAGCGGACGGUUUUGU |
| DS20-183A | 183 | caaaaccgtccgctctgcagca | 755 | AAACCGUCCGCUCUGCAGCA | 1041 | UGCUGCAGAGCGGACGGUUUUG |
| DS20-184A | 184 | aaaaccgtccgctctgcagcac | 756 | AACCGUCCGCUCUGCAGCAC | 1042 | GUGCUGCAGAGCGGACGGUUUU |
| DS20-185A | 185 | aaaccgtccgctctgcagcaca | 757 | ACCGUCCGCUCUGCAGCACA | 1043 | UGUGCUGCAGAGCGGACGGUUU |
| DS20-186A | 186 | aaccgtccgctctgcagcacag | 758 | CCGUCCGCUCUGCAGCACAG | 1044 | CUGUGCUGCAGAGCGGACGGUU |
| DS20-187A | 187 | accgtccgctctgcagcacagc | 759 | CGUCCGCUCUGCAGCACAGC | 1045 | GCUGUGCUGCAGAGCGGACGGU |
| DS20-188A | 188 | ccgtccgctctgcagcacagct | 760 | GUCCGCUCUGCAGCACAGCU | 1046 | AGCUGUGCUGCAGAGCGGACGG |
| DS20-189A | 189 | cgtccgctctgcagcacagctg | 761 | UCCGCUCUGCAGCACAGCUG | 1047 | CAGCUGUGCUGCAGAGCGGACG |
| DS20-190A | 190 | gtccgctctgcagcacagctgg | 762 | CCGCUCUGCAGCACAGCUGG | 1048 | CCAGCUGUGCUGCAGAGCGGAC |
| DS20-191A | 191 | tccgctctgcagcacagctggg | 763 | CGCUCUGCAGCACAGCUGGG | 1049 | CCCAGCUGUGCUGCAGAGCGGA |
| DS20-192A | 192 | cgctctgcagcacagctggggt | 764 | CUCUGCAGCACAGCUGGGGU | 1050 | ACCCCAGCUGUGCUGCAGAGCG |
| DS20-193A | 193 | gctctgcagcacagctggggtc | 765 | UCUGCAGCACAGCUGGGGUC | 1051 | GACCCCAGCUGUGCUGCAGAGC |
| DS20-194A | 194 | ctctgcagcacagctggggtca | 766 | CUGCAGCACAGCUGGGGUCA | 1052 | UGACCCCAGCUGUGCUGCAGAG |
| DS20-195A | 195 | tctgcagcacagctggggtcac | 767 | UGCAGCACAGCUGGGGUCAC | 1053 | GUGACCCCAGCUGUGCUGCAGA |
| DS20-196A | 196 | ctgcagcacagctggggtcacc | 768 | GCAGCACAGCUGGGGUCACC | 1054 | GGUGACCCCAGCUGUGCUGCAG |
| DS20-197A | 197 | tgcagcacagctggggtcacct | 769 | CAGCACAGCUGGGGUCACCU | 1055 | AGGUGACCCCAGCUGUGCUGCA |
| DS20-198A | 198 | gcagcacagctggggtcacctg | 770 | AGCACAGCUGGGGUCACCUG | 1056 | CAGGUGACCCCAGCUGUGCUGC |
| DS20-199A | 199 | cagcacagctggggtcacctga | 771 | GCACAGCUGGGGUCACCUGA | 1057 | UCAGGUGACCCCAGCUGUGCUG |
| DS20-200A | 200 | agcacagctggggtcacctgac | 772 | CACAGCUGGGGUCACCUGAC | 1058 | GUCAGGUGACCCCAGCUGUGCU |
| DS20-201A | 201 | gcacagctggggtcacctgacc | 773 | ACAGCUGGGGUCACCUGACC | 1059 | GGUCAGGUGACCCCAGCUGUGC |
| DS20-202A | 202 | cacagctggggtcacctgacct | 774 | CAGCUGGGGUCACCUGACCU | 1060 | AGGUCAGGUGACCCCAGCUGUG |
| DS20-203A | 203 | acagctggggtcacctgacctt | 775 | AGCUGGGGUCACCUGACCUU | 1061 | AAGGUCAGGUGACCCCAGCUGU |
| DS20-204A | 204 | cagctggggtcacctgaccttt | 776 | GCUGGGGUCACCUGACCUUU | 1062 | AAAGGUCAGGUGACCCCAGCUG |
| DS20-205A | 205 | agctggggtcacctgacctttc | 777 | CUGGGGUCACCUGACCUUUC | 1063 | GAAAGGUCAGGUGACCCCAGCU |
| DS20-206A | 206 | gctggggtcacctgacctttct | 778 | UGGGGUCACCUGACCUUUCU | 1064 | AGAAAGGUCAGGUGACCCCAGC |
| DS20-207A | 207 | ctggggtcacctgacctttctc | 779 | GGGGUCACCUGACCUUUCUC | 1065 | GAGAAAGGUCAGGUGACCCCAG |
| DS20-208A | 208 | tggggtcacctgacctttctcc | 780 | GGGUCACCUGACCUUUCUCC | 1066 | GGAGAAAGGUCAGGUGACCCCA |
| DS20-209A | 209 | ggggtcacctgacctttctcct | 781 | GGUCACCUGACCUUUCUCCU | 1067 | AGGAGAAAGGUCAGGUGACCCC |
| DS20-210A | 210 | gggtcacctgacctttctcctg | 782 | GUCACCUGACCUUUCUCCUG | 1068 | CAGGAGAAAGGUCAGGUGACCC |
| DS20-211A | 211 | ggtcacctgacctttctcctgt | 783 | UCACCUGACCUUUCUCCUGU | 1069 | ACAGGAGAAAGGUCAGGUGACC |
| DS20-212A | 212 | gtcacctgacctttctcctgtc | 784 | CACCUGACCUUUCUCCUGUC | 1070 | GACAGGAGAAAGGUCAGGUGAC |
| DS20-213A | 213 | tcacctgacctttctcctgtcc | 785 | ACCUGACCUUUCUCCUGUCC | 1071 | GGACAGGAGAAAGGUCAGGUGA |
| DS20-214A | 214 | cacctgacctttctcctgtccc | 786 | CCUGACCUUUCUCCUGUCCC | 1072 | GGGACAGGAGAAAGGUCAGGUG |
| DS20-215A | 215 | ccccacttgagctcagtggctg | 787 | CCACUUGAGCUCAGUGGCUG | 1073 | CAGCCACUGAGCUCAAGUGGGG |
| DS20-216A | 216 | cccacttgagctcagtggctgg | 788 | CACUUGAGCUCAGUGGCUGG | 1074 | CCAGCCACUGAGCUCAAGUGGG |
| DS20-217A | 217 | ccacttgagctcagtggctggg | 789 | ACUUGAGCUCAGUGGCUGGG | 1075 | CCCAGCCACUGAGCUCAAGUGG |
| DS20-218A | 218 | cacttgagctcagtggctgggc | 790 | CUUGAGCUCAGUGGCUGGGC | 1076 | GCCCAGCCACUGAGCUCAAGUG |
| DS20-219A | 219 | acttgagctcagtggctgggca | 791 | UUGAGCUCAGUGGCUGGGCA | 1077 | UGCCCAGCCACUGAGCUCAAGU |
| DS20-220A | 220 | cttgagctcagtggctgggcag | 792 | UGAGCUCAGUGGCUGGGCAG | 1078 | CUGCCCAGCCACUGAGCUCAAG |
| DS20-221A | 221 | ttgagctcagtggctgggcagc | 793 | GAGCUCAGUGGCUGGGCAGC | 1079 | GCUGCCCAGCCACUGAGCUCAA |
| DS20-222A | 222 | tgagctcagtggctgggcagca | 794 | AGCUCAGUGGCUGGGCAGCA | 1080 | UGCUGCCCAGCCACUGAGCUCA |
| DS20-223A | 223 | gagctcagtggctgggcagcag | 795 | GCUCAGUGGCUGGGCAGCAG | 1081 | CUGCUGCCCAGCCACUGAGCUC |
| DS20-224A | 224 | agctcagtggctgggcagcagg | 796 | CUCAGUGGCUGGGCAGCAGG | 1082 | CCUGCUGCCCAGCCACUGAGCU |
| DS20-225A | 225 | tcagtggctgggcagcagggga | 797 | AGUGGCUGGGCAGCAGGGGA | 1083 | UCCCCUGCUGCCCAGCCACUGA |
| DS20-226A | 226 | cagtggctgggcagcaggggat | 798 | GUGGCUGGGCAGCAGGGGAU | 1084 | AUCCCCUGCUGCCCAGCCACUG |
| DS20-227A | 227 | agtggctgggcagcaggggatg | 799 | UGGCUGGGCAGCAGGGGAUG | 1085 | CAUCCCCUGCUGCCCAGCCACU |
| DS20-228A | 228 | tggctgggcagcaggggatgca | 800 | GCUGGGCAGCAGGGGAUGCA | 1086 | UGCAUCCCCUGCUGCCCAGCCA |
| DS20-229A | 229 | ggctgggcagcaggggatgcat | 801 | CUGGGCAGCAGGGGAUGCAU | 1087 | AUGCAUCCCCUGCUGCCCAGCC |
| DS20-230A | 230 | gctgggcagcaggggatgcatg | 802 | UGGGCAGCAGGGGAUGCAUG | 1088 | CAUGCAUCCCCUGCUGCCCAGC |
| DS20-231A | 231 | ctgggcagcaggggatgcatgg | 803 | GGGCAGCAGGGGAUGCAUGG | 1089 | CCAUGCAUCCCCUGCUGCCCAG |
| DS20-232A | 232 | tgggcagcaggggatgcatggc | 804 | GGCAGCAGGGGAUGCAUGGC | 1090 | GCCAUGCAUCCCCUGCUGCCCA |
| DS20-233A | 233 | ggcagcaggggatgcatggcca | 805 | CAGCAGGGGAUGCAUGGCCA | 1091 | UGGCCAUGCAUCCCCUGCUGCC |
| DS20-234A | 234 | gcagcaggggatgcatggccac | 806 | AGCAGGGGAUGCAUGGCCAC | 1092 | GUGGCCAUGCAUCCCCUGCUGC |
| DS20-235A | 235 | cagcaggggatgcatggccact | 807 | GCAGGGGAUGCAUGGCCACU | 1093 | AGUGGCCAUGCAUCCCCUGCUG |
| DS20-236A | 236 | agcaggggatgcatggccactg | 808 | CAGGGGAUGCAUGGCCACUG | 1094 | CAGUGGCCAUGCAUCCCCUGCU |
| DS20-237A | 237 | gcaggggatgcatggccactgg | 809 | AGGGGAUGCAUGGCCACUGG | 1095 | CCAGUGGCCAUGCAUCCCCUGC |
| DS20-238A | 238 | caggggatgcatggccactggc | 810 | GGGGAUGCAUGGCCACUGGC | 1096 | GCCAGUGGCCAUGCAUCCCCUG |
| DS20-239A | 239 | aggggatgcatggccactggcc | 811 | GGGAUGCAUGGCCACUGGCC | 1097 | GGCCAGUGGCCAUGCAUCCCCU |
| DS20-240A | 240 | atgcatggccactggccggcca | 812 | GCAUGGCCACUGGCCGGCCA | 1098 | UGGCCGGCCAGUGGCCAUGCAU |
| DS20-241A | 241 | cggccaggtgcagctctcagct | 813 | GCCAGGUGCAGCUCUCAGCU | 1099 | AGCUGAGAGCUGCACCUGGCCG |
| DS20-242A | 242 | ggccaggtgcagctctcagctg | 814 | CCAGGUGCAGCUCUCAGCUG | 1100 | CAGCUGAGAGCUGCACCUGGCC |
| DS20-243A | 243 | gccaggtgcagctctcagctgg | 815 | CAGGUGCAGCUCUCAGCUGG | 1101 | CCAGCUGAGAGCUGCACCUGGC |
| DS20-244A | 244 | ccaggtgcagctctcagctggg | 816 | AGGUGCAGCUCUCAGCUGGG | 1102 | CCCAGCUGAGAGCUGCACCUGG |
| DS20-245A | 245 | caggtgcagctctcagctgggg | 817 | GGUGCAGCUCUCAGCUGGGG | 1103 | CCCCAGCUGAGAGCUGCACCUG |
| DS20-246A | 246 | aggtgcagctctcagctggggt | 818 | GUGCAGCUCUCAGCUGGGGU | 1104 | ACCCCAGCUGAGAGCUGCACCU |
| DS20-247A | 247 | ggtgcagctctcagctggggtg | 819 | UGCAGCUCUCAGCUGGGGUG | 1105 | CACCCCAGCUGAGAGCUGCACC |
| DS20-248A | 248 | gtgcagctctcagctggggtgt | 820 | GCAGCUCUCAGCUGGGGUGU | 1106 | ACACCCCAGCUGAGAGCUGCAC |
| DS20-249A | 249 | tgcagctctcagctggggtgtt | 821 | CAGCUCUCAGCUGGGGUGUU | 1107 | AACACCCCAGCUGAGAGCUGCA |
| DS20-250A | 250 | gcagctctcagctggggtgttc | 822 | AGCUCUCAGCUGGGGUGUUC | 1108 | GAACACCCCAGCUGAGAGCUGC |
| DS20-251A | 251 | cagctctcagctggggtgttca | 823 | GCUCUCAGCUGGGGUGUUCA | 1109 | UGAACACCCCAGCUGAGAGCUG |
| DS20-252A | 252 | agctctcagctggggtgttcag | 824 | CUCUCAGCUGGGGUGUUCAG | 1110 | CUGAACACCCCAGCUGAGAGCU |
| DS20-253A | 253 | gctctcagctggggtgttcaga | 825 | UCUCAGCUGGGGUGUUCAGA | 1111 | UCUGAACACCCCAGCUGAGAGC |
| DS20-254A | 254 | ctctcagctggggtgttcagag | 826 | CUCAGCUGGGGUGUUCAGAG | 1112 | CUCUGAACACCCCAGCUGAGAG |
| DS20-255A | 255 | tctcagctggggtgttcagagg | 827 | UCAGCUGGGGUGUUCAGAGG | 1113 | CCUCUGAACACCCCAGCUGAGA |
| DS20-256A | 256 | ctcagctggggtgttcagagga | 828 | CAGCUGGGGUGUUCAGAGGA | 1114 | UCCUCUGAACACCCCAGCUGAG |
| DS20-257A | 257 | tcagctggggtgttcagaggac | 829 | AGCUGGGGUGUUCAGAGGAC | 1115 | GUCCUCUGAACACCCCAGCUGA |
| DS20-258A | 258 | cagctggggtgttcagaggacg | 830 | GCUGGGGUGUUCAGAGGACG | 1116 | CGUCCUCUGAACACCCCAGCUG |
| DS20-259A | 259 | agctggggtgttcagaggacgc | 831 | CUGGGGUGUUCAGAGGACGC | 1117 | GCGUCCUCUGAACACCCCAGCU |
| DS20-260A | 260 | gctggggtgttcagaggacgcc | 832 | UGGGGUGUUCAGAGGACGCC | 1118 | GGCGUCCUCUGAACACCCCAGC |
| DS20-261A | 261 | ctggggtgttcagaggacgcct | 833 | GGGGUGUUCAGAGGACGCCU | 1119 | AGGCGUCCUCUGAACACCCCAG |
| DS20-262A | 262 | tggggtgttcagaggacgcctg | 834 | GGGUGUUCAGAGGACGCCUG | 1120 | CAGGCGUCCUCUGAACACCCCA |
| DS20-263A | 263 | ggggtgttcagaggacgcctgt | 835 | GGUGUUCAGAGGACGCCUGU | 1121 | ACAGGCGUCCUCUGAACACCCC |
| DS20-264A | 264 | gggtgttcagaggacgcctgtg | 836 | GUGUUCAGAGGACGCCUGUG | 1122 | CACAGGCGUCCUCUGAACACCC |
| DS20-265A | 265 | ggtgttcagaggacgcctgtgt | 837 | UGUUCAGAGGACGCCUGUGU | 1123 | ACACAGGCGUCCUCUGAACACC |
| DS20-266A | 266 | gtgttcagaggacgcctgtgtc | 838 | GUUCAGAGGACGCCUGUGUC | 1124 | GACACAGGCGUCCUCUGAACAC |
| DS20-267A | 267 | tgttcagaggacgcctgtgtcc | 839 | UUCAGAGGACGCCUGUGUCC | 1125 | GGACACAGGCGUCCUCUGAACA |
| DS20-268A | 268 | gttcagaggacgcctgtgtcct | 840 | UCAGAGGACGCCUGUGUCCU | 1126 | AGGACACAGGCGUCCUCUGAAC |
| DS20-269A | 269 | ttcagaggacgcctgtgtcctc | 841 | CAGAGGACGCCUGUGUCCUC | 1127 | GAGGACACAGGCGUCCUCUGAA |
| DS20-270A | 270 | tcagaggacgcctgtgtcctcc | 842 | AGAGGACGCCUGUGUCCUCC | 1128 | GGAGGACACAGGCGUCCUCUGA |
| DS20-271A | 271 | cagaggacgcctgtgtcctccc | 843 | GAGGACGCCUGUGUCCUCCC | 1129 | GGGAGGACACAGGCGUCCUCUG |
| DS20-272A | 272 | agaggacgcctgtgtcctcccc | 844 | AGGACGCCUGUGUCCUCCCC | 1130 | GGGGAGGACACAGGCGUCCUCU |
| DS20-273A | 273 | gaggacgcctgtgtcctcccct | 845 | GGACGCCUGUGUCCUCCCCU | 1131 | AGGGGAGGACACAGGCGUCCUC |
| DS20-274A | 274 | aggacgcctgtgtcctcccctc | 846 | GACGCCUGUGUCCUCCCCUC | 1132 | GAGGGGAGGACACAGGCGUCCU |
| DS20-275A | 275 | ctctgtcacccttggaggcaga | 847 | CUGUCACCCUUGGAGGCAGA | 1133 | UCUGCCUCCAAGGGUGACAGAG |
| DS20-276A | 276 | tctgtcacccttggaggcagag | 848 | UGUCACCCUUGGAGGCAGAG | 1134 | CUCUGCCUCCAAGGGUGACAGA |
| DS20-277A | 277 | ctgtcacccttggaggcagaga | 849 | GUCACCCUUGGAGGCAGAGA | 1135 | UCUCUGCCUCCAAGGGUGACAG |
| DS20-278A | 278 | tgtcacccttggaggcagagaa | 850 | UCACCCUUGGAGGCAGAGAA | 1136 | UUCUCUGCCUCCAAGGGUGACA |
| DS20-279A | 279 | gtcacccttggaggcagagaac | 851 | CACCCUUGGAGGCAGAGAAC | 1137 | GUUCUCUGCCUCCAAGGGUGAC |
| DS20-280A | 280 | tcacccttggaggcagagaact | 852 | ACCCUUGGAGGCAGAGAACU | 1138 | AGUUCUCUGCCUCCAAGGGUGA |
| DS20-281A | 281 | cacccttggaggcagagaactt | 853 | CCCUUGGAGGCAGAGAACUU | 1139 | AAGUUCUCUGCCUCCAAGGGUG |
| DS20-282A | 282 | acccttggaggcagagaacttt | 854 | CCUUGGAGGCAGAGAACUUU | 1140 | AAAGUUCUCUGCCUCCAAGGGU |
| DS20-283A | 283 | cccttggaggcagagaactttg | 855 | CUUGGAGGCAGAGAACUUUG | 1141 | CAAAGUUCUCUGCCUCCAAGGG |
| DS20-284A | 284 | ccttggaggcagagaactttgc | 856 | UUGGAGGCAGAGAACUUUGC | 1142 | GCAAAGUUCUCUGCCUCCAAGG |
| DS20-285A | 285 | cttggaggcagagaactttgcc | 857 | UGGAGGCAGAGAACUUUGCC | 1143 | GGCAAAGUUCUCUGCCUCCAAG |
| DS20-286A | 286 | ttggaggcagagaactttgccc | 858 | GGAGGCAGAGAACUUUGCCC | 1144 | GGGCAAAGUUCUCUGCCUCCAA |

Note:
lower case, DNA; upper case, RNA. Target sequence is identical to the identified sense sequence but the nucleotide “U” is converted to “T”.

TABLE 1.2
Activity of the saRNAs in increasing FVII mRNA expression

| | Relative FVII | | Relative FVII | | Relative FVII | | Relative FVII |
|---|---|---|---|---|---|---|---|
| | mRNA expression | | mRNA expression | | mRNA expression | | mRNA expression |
| saRNA name | (fold change) | saRNA name | (fold change) | saRNA name | (fold change) | saRNA name | (fold change) |
| DS20-259S | 3.03 | DS20-011S | 1.59 | DS20-272A | 1.34 | DS20-169B | 1.22 |
| DS20-082A | 2.96 | DS20-173B | 1.58 | DS20-278S | 1.34 | DS20-051S | 1.22 |
| DS20-252A | 2.85 | DS20-151S | 1.58 | DS20-210B | 1.34 | DS20-175S | 1.22 |
| DS20-086A | 2.70 | DS20-042B | 1.57 | DS20-215B | 1.34 | DS20-155B | 1.22 |
| DS20-241A | 2.63 | DS20-028S | 1.56 | DS20-282A | 1.34 | DS20-157B | 1.22 |
| DS20-207B | 2.42 | DS20-121B | 1.56 | DS20-091A | 1.34 | DS20-061S | 1.21 |
| DS20-205B | 2.30 | DS20-061B | 1.56 | DS20-154A | 1.34 | DS20-064S | 1.21 |
| DS20-205S | 2.27 | DS20-041B | 1.55 | DS20-079B | 1.34 | DS20-140B | 1.21 |
| DS20-027A | 2.27 | DS20-178B | 1.55 | DS20-045A | 1.34 | DS20-103S | 1.21 |
| DS20-086B | 2.24 | DS20-040S | 1.55 | DS20-093B | 1.34 | DS20-226A | 1.20 |
| DS20-146B | 2.22 | DS20-252B | 1.54 | DS20-056A | 1.34 | DS20-058B | 1.20 |
| DS20-029S | 2.19 | DS20-241B | 1.53 | DS20-052B | 1.34 | DS20-074A | 1.20 |
| DS20-010A | 2.16 | DS20-069A | 1.53 | DS20-098A | 1.34 | DS20-118S | 1.20 |
| DS20-151B | 2.14 | DS20-117A | 1.53 | DS20-044A | 1.34 | DS20-124A | 1.20 |
| DS20-124B | 2.07 | DS20-117S | 1.53 | DS20-125S | 1.33 | DS20-175A | 1.20 |
| DS20-218B | 2.06 | DS20-084A | 1.53 | DS20-188A | 1.33 | DS20-099A | 1.20 |
| DS20-261S | 2.04 | DS20-145B | 1.52 | DS20-127A | 1.33 | DS20-044B | 1.20 |
| DS20-055B | 2.04 | DS20-030S | 1.52 | DS20-093S | 1.33 | DS20-104B | 1.19 |
| DS20-081A | 2.01 | DS20-054S | 1.52 | DS20-204A | 1.33 | DS20-177B | 1.19 |
| DS20-242A | 2.01 | DS20-226S | 1.52 | DS20-220S | 1.33 | DS20-128S | 1.19 |
| DS20-156S | 1.98 | DS20-063S | 1.51 | DS20-088B | 1.32 | DS20-046S | 1.19 |
| DS20-207S | 1.98 | DS20-203A | 1.51 | DS20-175B | 1.32 | DS20-016B | 1.19 |
| DS20-038B | 1.97 | DS20-228S | 1.51 | DS20-126B | 1.32 | DS20-017B | 1.18 |
| DS20-124S | 1.96 | DS20-276B | 1.51 | DS20-189B | 1.32 | DS20-178A | 1.18 |
| DS20-218S | 1.96 | DS20-098B | 1.51 | DS20-043B | 1.32 | DS20-037A | 1.18 |
| DS20-055A | 1.94 | DS20-150B | 1.51 | DS20-174S | 1.32 | DS20-220B | 1.18 |
| DS20-145A | 1.94 | DS20-217B | 1.50 | DS20-038S | 1.32 | DS20-148B | 1.18 |
| DS20-206A | 1.93 | DS20-005A | 1.50 | DS20-207A | 1.31 | DS20-032B | 1.18 |
| DS20-029B | 1.93 | DS20-028A | 1.50 | DS20-111S | 1.31 | DS20-106B | 1.18 |
| DS20-055S | 1.91 | DS20-228A | 1.50 | DS20-061A | 1.31 | DS20-098S | 1.18 |
| DS20-286A | 1.91 | DS20-093A | 1.50 | DS20-089B | 1.31 | DS20-177S | 1.18 |
| DS20-206B | 1.91 | DS20-115B | 1.50 | DS20-094S | 1.31 | DS20-019A | 1.18 |
| DS20-087A | 1.90 | DS20-083B | 1.49 | DS20-136B | 1.31 | DS20-031B | 1.18 |
| DS20-151A | 1.89 | DS20-271A | 1.49 | DS20-214B | 1.31 | DS20-148A | 1.17 |
| DS20-085B | 1.89 | DS20-113B | 1.49 | DS20-182B | 1.31 | DS20-216B | 1.17 |
| DS20-082B | 1.87 | DS20-202B | 1.48 | DS20-115A | 1.31 | DS20-179A | 1.17 |
| DS20-117B | 1.86 | DS20-147B | 1.48 | DS20-122A | 1.31 | DS20-202S | 1.17 |
| DS20-027B | 1.86 | DS20-101B | 1.48 | DS20-036B | 1.31 | DS20-127S | 1.17 |
| DS20-114B | 1.85 | DS20-078A | 1.47 | DS20-158B | 1.31 | DS20-138A | 1.17 |
| DS20-146A | 1.85 | DS20-110A | 1.47 | DS20-112B | 1.30 | DS20-226B | 1.17 |
| DS20-228B | 1.84 | DS20-110S | 1.47 | DS20-196S | 1.30 | DS20-046A | 1.17 |
| DS20-206S | 1.84 | DS20-276S | 1.47 | DS20-082S | 1.30 | DS20-277B | 1.17 |
| DS20-053B | 1.84 | DS20-046B | 1.47 | DS20-121A | 1.30 | DS20-036S | 1.16 |
| DS20-284A | 1.83 | DS20-052A | 1.46 | DS20-084B | 1.30 | DS20-096S | 1.16 |
| DS20-146S | 1.83 | DS20-285S | 1.46 | DS20-194B | 1.30 | DS20-261A | 1.16 |
| DS20-114S | 1.83 | DS20-135S | 1.46 | DS20-034A | 1.30 | DS20-253B | 1.16 |
| DS20-123B | 1.82 | DS20-006S | 1.45 | DS20-150S | 1.29 | DS20-035S | 1.16 |
| DS20-083A | 1.82 | DS20-036A | 1.45 | DS20-037S | 1.29 | DS20-109B | 1.16 |
| DS20-075A | 1.82 | DS20-135B | 1.44 | DS20-217A | 1.29 | DS20-227B | 1.16 |
| DS20-188B | 1.80 | DS20-012A | 1.44 | DS20-244S | 1.29 | DS20-276A | 1.16 |
| DS20-062B | 1.80 | DS20-174B | 1.44 | DS20-255A | 1.28 | DS20-015B | 1.16 |
| DS20-286S | 1.79 | DS20-040B | 1.44 | DS20-218A | 1.28 | DS20-025B | 1.15 |
| DS20-060B | 1.79 | DS20-060A | 1.43 | DS20-258S | 1.28 | DS20-243S | 1.15 |
| DS20-261B | 1.78 | DS20-051B | 1.43 | DS20-081B | 1.28 | DS20-079S | 1.15 |
| DS20-038A | 1.78 | DS20-086S | 1.43 | DS20-076A | 1.28 | DS20-072B | 1.15 |
| DS20-110B | 1.77 | DS20-181S | 1.43 | DS20-236A | 1.28 | DS20-253A | 1.15 |
| DS20-285A | 1.77 | DS20-009B | 1.43 | DS20-051A | 1.27 | DS20-211A | 1.15 |
| DS20-205A | 1.77 | DS20-170B | 1.43 | DS20-106A | 1.27 | DS20-054A | 1.15 |
| DS20-260S | 1.77 | DS20-198B | 1.42 | DS20-095B | 1.27 | DS20-120A | 1.14 |
| DS20-004A | 1.76 | DS20-035A | 1.42 | DS20-181B | 1.27 | DS20-213B | 1.14 |
| DS20-004B | 1.75 | DS20-059B | 1.42 | DS20-180S | 1.27 | DS20-083S | 1.14 |
| DS20-286B | 1.74 | DS20-053S | 1.42 | DS20-241S | 1.27 | DS20-149S | 1.14 |
| DS20-122B | 1.73 | DS20-164S | 1.42 | DS20-239A | 1.27 | DS20-178S | 1.14 |
| DS20-204B | 1.73 | DS20-003B | 1.42 | DS20-190S | 1.26 | DS20-172B | 1.14 |
| DS20-088S | 1.73 | DS20-203S | 1.42 | DS20-202A | 1.26 | DS20-190A | 1.14 |
| DS20-030B | 1.72 | DS20-056B | 1.42 | DS20-091B | 1.26 | DS20-197A | 1.13 |
| DS20-087B | 1.72 | DS20-035B | 1.42 | DS20-084S | 1.26 | DS20-190B | 1.13 |
| DS20-127B | 1.72 | DS20-002B | 1.41 | DS20-045B | 1.26 | DS20-139B | 1.13 |
| DS20-077B | 1.71 | DS20-039A | 1.41 | DS20-263S | 1.25 | DS20-023B | 1.13 |
| DS20-085S | 1.71 | DS20-147S | 1.41 | DS20-005S | 1.25 | DS20-215S | 1.13 |
| DS20-125B | 1.70 | DS20-235B | 1.41 | DS20-129B | 1.25 | DS20-255B | 1.13 |
| DS20-116B | 1.70 | DS20-076B | 1.41 | DS20-101A | 1.25 | DS20-133S | 1.13 |
| DS20-119B | 1.70 | DS20-053A | 1.41 | DS20-059A | 1.25 | DS20-219B | 1.13 |
| DS20-009A | 1.70 | DS20-215A | 1.41 | DS20-260B | 1.25 | DS20-014B | 1.13 |
| DS20-123S | 1.69 | DS20-003A | 1.41 | DS20-116S | 1.25 | DS20-074B | 1.13 |
| DS20-119S | 1.69 | DS20-118B | 1.41 | DS20-060S | 1.25 | DS20-107S | 1.13 |
| DS20-154B | 1.68 | DS20-204S | 1.40 | DS20-230S | 1.25 | DS20-237S | 1.12 |
| DS20-037B | 1.68 | DS20-274B | 1.40 | DS20-039B | 1.24 | DS20-155A | 1.12 |
| DS20-107B | 1.68 | DS20-062S | 1.39 | DS20-099B | 1.24 | DS20-225A | 1.12 |
| DS20-211B | 1.67 | DS20-050B | 1.39 | DS20-092B | 1.24 | DS20-173A | 1.12 |
| DS20-002A | 1.66 | DS20-095S | 1.39 | DS20-095A | 1.24 | DS20-219S | 1.12 |
| DS20-120S | 1.66 | DS20-041A | 1.38 | DS20-023A | 1.24 | DS20-025A | 1.12 |
| DS20-085A | 1.66 | DS20-217S | 1.38 | DS20-192B | 1.24 | DS20-132S | 1.12 |
| DS20-114A | 1.66 | DS20-092A | 1.38 | DS20-260A | 1.24 | DS20-152B | 1.12 |
| DS20-077S | 1.65 | DS20-078B | 1.38 | DS20-232A | 1.24 | DS20-283A | 1.11 |
| DS20-107A | 1.65 | DS20-050A | 1.38 | DS20-227A | 1.24 | DS20-071A | 1.11 |
| DS20-077A | 1.64 | DS20-243B | 1.38 | DS20-094B | 1.24 | DS20-208B | 1.11 |
| DS20-054B | 1.64 | DS20-108A | 1.38 | DS20-239B | 1.24 | DS20-030A | 1.11 |
| DS20-274S | 1.64 | DS20-238B | 1.38 | DS20-007B | 1.24 | DS20-072A | 1.11 |
| DS20-203B | 1.63 | DS20-179B | 1.37 | DS20-273B | 1.23 | DS20-080S | 1.11 |
| DS20-254S | 1.63 | DS20-273A | 1.37 | DS20-262S | 1.23 | DS20-073B | 1.11 |
| DS20-242B | 1.63 | DS20-259B | 1.36 | DS20-198A | 1.23 | DS20-094A | 1.11 |
| DS20-121S | 1.63 | DS20-123A | 1.36 | DS20-227S | 1.23 | DS20-238S | 1.11 |
| DS20-010B | 1.63 | DS20-235A | 1.36 | DS20-274A | 1.23 | DS20-210S | 1.11 |
| DS20-010S | 1.62 | DS20-240A | 1.36 | DS20-119A | 1.23 | DS20-257S | 1.10 |
| DS20-120B | 1.61 | DS20-042A | 1.36 | DS20-173S | 1.23 | DS20-043A | 1.10 |
| DS20-116A | 1.60 | DS20-006B | 1.35 | DS20-238A | 1.23 | DS20-109A | 1.10 |
| DS20-254B | 1.60 | DS20-019B | 1.35 | DS20-056S | 1.23 | DS20-266S | 1.10 |
| DS20-079A | 1.59 | DS20-026B | 1.35 | DS20-285B | 1.22 | DS20-126S | 1.10 |
| DS20-028B | 1.59 | DS20-087S | 1.35 | DS20-007A | 1.22 | DS20-179S | 1.10 |
| DS20-005B | 1.59 | DS20-011A | 1.35 | DS20-103B | 1.22 | DS20-250S | 1.10 |

### EQUIVALENTS

While specific embodiments of the compositions and methods herein have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

### INCORPORATION BY REFERENCE

All publications, patents, and accession numbers mentioned herein are hereby incorporated by reference in their entirety as if each individual publication or patent was specifically and individually indicated to be incorporated by reference.

### REFERENCES

1. Heinz S, Braspenning J. Measurement of blood coagulation factor synthesis in cultures of human hepatocytes. Methods in molecular biology. 2015; 1250: 309-316.
2. Yang L, Li Y, Bhattacharya A, Zhang Y. A plasma proteolysis pathway comprising blood coagulation proteases. Oncotarget. 2016; 7(27): 40919-40938.
3. O'Hara P J, Grant F J, Haldeman B A, Gray C L, Insley M Y, Hagen F S, Murray M J. Nucleotide sequence of the gene coding for human factor VII, a vitamin K-dependent protein participating in blood coagulation. Proceedings of the national academy of sciences of the United States of America. 1987; 84(15): 5158-5162.
4. Barbon E, Pignani S, Branchini A, Bernardi F, Pinotti M, Bovolenta M. An engineered tale-transcription factor rescues transcription of factor VII impaired by promoter mutations and enhances its endogenous expression in hepatocytes. Scientific reports. 2016; 6: 28304.
5. Long-Cheng Li, Steven T Okino, Hong Zhao, Deepa Pookot, Robert F Place, Shinji Urakami, Hideki Enokida, Rajvir Dahiya. Small dsRNAs induce transcriptional activation in human cells. PNAS. 2006 Nov. 14; 103(46): 17337-42.
6. Robert F Place, Ji Wang, Emily J Noonan, Rachel Meyers, Muthiah Manoharan, Klaus Charisse, Rick Duncan, Vera Huang, Xiaoling Wang, Long-Cheng Li. Formulation of small activating RNA into lipidoid nanoparticles inhibits xenograft prostate tumor growth by inducing p21 expression. Mol Ther Nucleic Acids. 2012 Mar. 27; 1(3):e15.

What is claimed is:

1. A small activating RNA (saRNA) comprising a sense strand and an antisense strand, wherein:
each of the sense strand or the antisense strand of the saRNA comprises a consecutive oligonucleotide sequence of 16 to 35 consecutive nucleotides, wherein the consecutive oligonucleotide sequence independently has at least 75%, at least 80%, at least 85%, at least 90%, at least 95%, at least 98% or 100% homology or complementarity to an equal length consecutive fragment of SEQ ID NO: 1437; and wherein
the saRNA increases the expression of FVII gene by at least 10% as compared to baseline expression of FVII gene.

2. The saRNA of claim 1, wherein the equal length consecutive fragment of SEQ ID NO: 1437 is located in a region upstream of the transcription start site (TSS) of FVII gene, and wherein the equal length consecutive fragment of SEQ ID NO: 1437 is located in a region selected from the group consisting of: region −557 to −379; region −346 to −298; region −271 to −91; and, region −96 to −1; and/or
the equal length consecutive fragment of SEQ ID NO: 1437 is located in a region of SEQ ID NO: 1438, SEQ ID NO: 1439, SEQ ID NO: 1440, or SEQ ID NO: 1441.

3. The saRNA of claim 1 or 2, wherein the equal length consecutive fragment of SEQ ID NO: 1437 is selected from SEQ ID NO: 1-286.

4. The saRNA of any one of claims 1-3, wherein the equal length consecutive fragment of SEQ ID NO: 1437 is selected from SEQ ID NOs: 37, 55, 77, 86, 116, 117, 120, 124, 145, 146, 151, 177, 188, 205, 207, 218, 228, 241, 242, 10, 11, 29, 88, 121, 156, 259, 261, 274, 9, 27, 69, 81, 82, 107, 240 and 252.

5. The saRNA of any one of claims 1-4, wherein the consecutive oligonucleotide sequence has (1) GC content between 40% and 70%; (2) no more than 5 consecutive identical nucleotides; (3) no more than 3 dinucleotide or trinucleotide repeats; and/or
the consecutive oligonucleotide sequence is complementary to the sense strand of the saRNA or to the antisense strand of the saRNA.

6. The saRNA of any one of claims 1-5, wherein the sense strand and the antisense strand independently has a length of about 16 to about 35, about 17 to about 30, about 18 to about 25, or about 19 to about 22 nucleotides.

7. The saRNA of any one of claims 1-6, wherein the sense strand and the antisense strand have a complementarity of at least 90%; and/or
wherein the sense strand and the antisense strand are located on two different nucleic acid strands or on a contiguous nucleic acid strand; and/or
wherein the consecutive oligonucleotide sequence comprises 0, 1, 2, or 3 mismatches to the complementary region of the sense strand or the complementary region of the antisense strand.

8. The saRNA of any one of claims 1-7, wherein the sense strand and the antisense strand each comprises a complementary region, and wherein the complementary regions of the sense strand and the antisense strand form a double-stranded nucleic acid structure.

9. The saRNA of claim 8, wherein the sense strand or the antisense strand comprises a 3′ overhang which is independently 1-6, 1-5, or 2-3 nucleotides in length; or
the double-stranded nucleic acid structure is blunt-ended.

10. The saRNA of claim 9, wherein at least one of the nucleotides of the overhang is thymine deoxyribonucleotide (dT).

11. The saRNA of any one of claims 1-10, wherein the consecutive oligonucleotide sequence of the sense strand has at least 75% sequence homology to a nucleotide sequence selected from SEQ ID NOs: 287-858, and/or
the consecutive oligonucleotide sequence of the antisense strand has at least 75% sequence homology to a nucleotide sequence selected from SEQ ID NOs: 859-1430.

12. The saRNA of any one of claims 1-11, wherein the consecutive oligonucleotide sequence of the sense strand is selected from SEQ ID NOs: 287-858, and the consecutive oligonucleotide sequence of the antisense strand is selected from SEQ ID NOs: 859-1430.

13. The saRNA of any one of claims 1-12, wherein when the consecutive oligonucleotide sequence of the sense strand is as set forth in SEQ ID NO: n, the consecutive oligonucleotide sequence of the antisense strand is as set forth in SEQ ID NO: n+572 or n+858, wherein n is an integer selected from 287-572; and/or,
wherein when the consecutive oligonucleotide sequence of the sense strand is as set forth in SEQ ID NO: n′, the consecutive oligonucleotide sequence of the antisense strand is as set forth in SEQ ID NO: n′+286, wherein n′ is an integer selected from 573-858.

14. The saRNA of any one of claims 1-13, wherein the sense strand comprises a consecutive nucleotide sequence selected from SEQ ID NOs: 323, 341, 363, 372, 402, 403, 406, 410, 431, 432, 437, 463, 474, 491, 493, 504, 514, 527, 528, 296, 297, 315, 374, 407, 442, 545, 547, 560, 581, 582, 599, 627, 641, 653, 654, 658, 679, 749, 812, 813, and 824; and/or
the antisense strand comprises a consecutive nucleotide sequence selected from SEQ ID NOs: 859, 913, 935, 944, 974, 975, 978, 982, 1003, 1004, 1009, 1035, 1046, 1063, 1065, 1076, 1086, 1099, 1100, 1154, 1155, 1173, 1232, 1265, 1268, 1300, 1349, 1351, 1362, 1372, 1385, 1403, 1405, 1418, 867, 868, 885, 927, 939, 940, 965, 1098, and 1110.

15. The saRNA of any one of claims 1-14, wherein the sense strand comprises a nucleotide sequence of SEQ ID NO: m, and the antisense strand comprises a nucleotide sequence of SEQ ID NO: m+572, and wherein m is selected from 323, 341, 363, 372, 402, 403, 406, 410, 431, 432, 437, 463, 474, 491, 493, 504, 514, 527, 528; and/or
wherein the sense strand comprises a nucleotide sequence of SEQ ID NO: m′, and the antisense strand comprises a nucleotide sequence of SEQ ID NO: m+858, and wherein m is selected from 296, 297, 315, 374, 407, 410, 442, 491, 493, 504, 514, 527, 545, 547, 560; and/or,
wherein the sense strand comprises a nucleotide sequence of SEQ ID NO: m″, and the antisense strand comprises a nucleotide sequence of SEQ ID NO: m″+286, and wherein m is selected from 581, 582, 599, 627, 641, 653, 654, 658, 679, 749, 812, 813, and 824.

16. The saRNA of any one of claims 1-16, wherein the sense strand comprises a consecutive nucleotide sequence selected from SEQ ID NOs: 1442, 1444, 1446, 1448, 1450, 1452, 1454, 1456, 1458, 1460, 1462, 1464, 1466, 1468, 1470, 1472, 1474 and 1476; and/or
the antisense strand comprises a consecutive nucleotide sequence selected from SEQ ID NOs: 1443, 1445, 1447, 1449, 1451, 1453, 1455, 1457, 1459, 1461, 1463, 1465, 1467, 1469, 1471, 1473 and 1475; and/or the saRNA comprises the sense strand and the antisense strand to form duplexes as listed in Table 11.

17. The saRNA of any one of claims 1-16, wherein at least one nucleotide of the saRNA is a chemically modified nucleotide.

18. The saRNA of claim 17, wherein the chemically modified nucleotide is a nucleotide with at least one the following modifications:
a) modification of a phosphodiester bond connecting nucleotides in the nucleotide sequence of the saRNA;
b) modification of 2′-OH of a ribose in the nucleotide sequence of the saRNA; and
c) modification of a base in the nucleotide sequence of the saRNA.

19. The saRNA of claim 18, wherein the modification of a phosphodiester bond connecting nucleotides is selected from a phosphorothioate modification and boranophosphate modification; and/or
the modified of 2′-OH is selected from the group consisting of 2′-fluoro modification, 2′-oxymethyl modification, 2′-oxyethylidene methoxy modification, 2,4′-dinitrophenol modification, 2′-amino modification and 2′-deoxy modification; and/or
the modification of a base is selected from the group consisting of 5′-bromouracil modification, 5′-iodouracil modification, N-methyluracil modification, and 2,6-diaminopurine modification.

20. The saRNA of claim 17, wherein at least one nucleotide of the saRNA is a locked nucleic acid, an abasic nucleotide, a 2′-amino-modified nucleotide, a 2′-alkyl-modified nucleotide, a morpholino nucleotide, a phosphoramidate, or a non-natural base comprising nucleotide; and/or
wherein the chemical modification of the at least one chemically modified nucleotide is an addition of a (E)-vinylphosphonate moiety at the 5′ end of the sense strand or the antisense strand.

21. An oligonucleotide modulator comprising one or more saRNA according to any one of claims 1-20.

22. The oligonucleotide modulator of claim 21, further comprising one or more moieties or components conjugated, combined or mixed with said saRNA(s).

23. The oligonucleotide modulator of claim 21, wherein the sense strand and/or the antisense strand of the saRNA is conjugated to one or more conjugation moieties selected from the group consisting of a lipid, a fatty acid (such as fatty acid comprising a carbon chain with 4-30, 12-24, or 16-22 carbon atoms), a fluorophore, a ligand, a saccharide, a peptide, and an antibody.

24. The oligonucleotide modulator of claim 22, wherein the conjugation moiety is independently selected from a lipid, a cell-penetrating peptide, a polyethylene glycol, an alkaloid, a tryptamine, a benzimidazole, a quinolone, an amino acid, a cholesterol, a glucose, a N-acetylgalactosamine, and any combinations thereof.

25. The oligonucleotide modulator of claim 22, wherein the conjugation moieties is independently selected from S9, tC2, tC2x6, C5x5, and any combinations thereof, 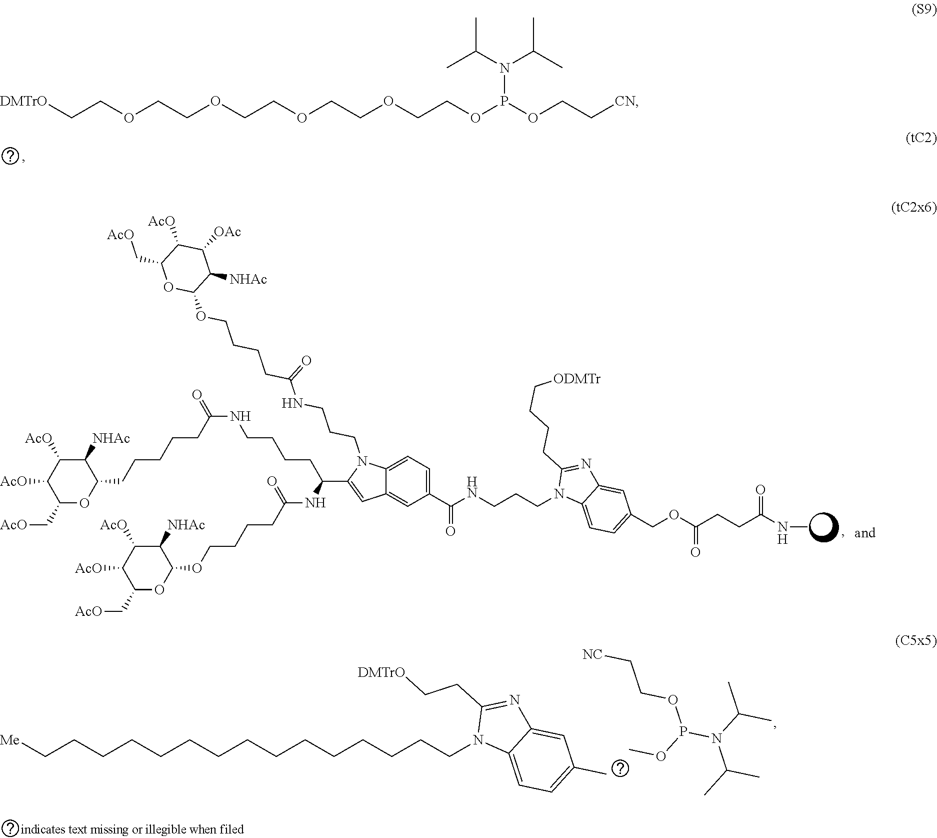
wherein [symbol] represents a support material.

26. The oligonucleotide modulator of claim 21, wherein the oligonucleotide modulator further comprises a saRNA conjugated to or combined with one or more of other active moieties for FVII related disease or condition or disorder treatment, wherein the one or more of other active moieties are independently selected from a small molecule chemical moiety, a polypeptide and an antibody.

27. An isolated oligonucleotide, wherein (a) the isolated oligonucleotide is a consecutive oligonucleotide sequence of 16-35 consecutive nucleotides having at least 75%, at least 80%, at least 85%, at least 90%, at least 95%, at least 98% or 100% homology to an equal length consecutive fragment of SEQ ID NO: 1437; or
wherein (b) the isolated oligonucleotide is a consecutive oligonucleotide sequence of 16-35 consecutive nucleotides having at least 75%, at least 80%, at least 85%, at least 90%, at least 95%, at least 98% or 100% complementarity to an equal length consecutive fragment of SEQ ID NO: 1437.

28. The isolated oligonucleotide of claim 27, wherein the equal length consecutive fragment of SEQ ID NO: 1437 is located in a region upstream of the transcription start site (TSS) of FVII gene, and wherein
the equal length consecutive fragment of SEQ ID NO: 1437 is located in a region selected from the group consisting of: region −557 to −379; region −346 to −298; region −271 to −91; and, region −96 to −1; and/or
the equal length consecutive fragment of SEQ ID NO: 1437 is located in a region of SEQ ID NO: 1438, SEQ ID NO: 1439, SEQ ID NO: 1440, or SEQ ID NO: 1441.

29. The isolated oligonucleotide of claim 27, wherein the isolated oligonucleotide (a) is a nucleic acid sequence selected from SEQ ID NOs: 1-286, such as selected from SEQ ID NOs: 37, 55, 77, 86, 116, 117, 120, 124, 145, 146, 151, 177, 188, 205, 207, 218, 228, 241, 242, 10, 11, 29, 88, 121, 156, 259, 261, 274, 9, 27, 69, 81, 82, 107, 240 and 252; or
the isolated oligonucleotide (b) is a nucleic acid sequence complementary to a nucleic acid sequence selected from SEQ ID NOs: 1-286, such as complementary to SEQ ID NOs: 37, 55, 77, 86, 116, 117, 120, 124, 145, 146, 151, 177, 188, 205, 207, 218, 228, 241, 242, 10, 11, 29, 88, 121, 156, 259, 261, 274, 9, 27, 69, 81, 82, 107, 240 and 252;

30. An oligonucleotide complex comprising:
the antisense strand of the saRNA of any of claim 1-20 and the sense strand of the isolated oligonucleotide (a) of any of claim 27-29; or
the sense strand of the saRNA of any of claim 1-20 and the antisense strand of the isolated oligonucleotide (b) of any of claim 27-29.

31. The oligonucleotide complex of claim 30, wherein the oligonucleotide complex activates the expression of FVII gene by at least 10% as compared to baseline expression of the FVII gene.

32. An isolated nucleic acid molecule, wherein at least 20% (such as at least 30%, at least 40%, at least 45%, at least 50%) of saRNAs designed to target the sequence of the isolated nucleic acid molecule activate the expression of FVII gene by at least 10%, wherein the designed saRNA has (1) GC content between 40% and 70%; (2) no more than 5 consecutive identical nucleotides; (3) no more than 3 dinucleotide or trinucleotide repeats.

33. An isolated nucleic acid molecule having a sequence corresponding to a region upstream of the transcription start site of the FVII gene, wherein the sequence is located on the region selected from region −557 to −379, region −346 to −298, region −271 to −91 and region −96 to −1 or any sub-regions in any of the above regions, upstream of the transcription start site of the FVII gene.

34. An isolated nucleic acid molecule having a sequence selected from SEQ ID NOs: 1438-1441.

35. The isolated nucleic acid molecule of any one of claims 32-34 comprising the isolated oligonucleotide of claim 27(a) or 29(a).

36. The isolated nucleic acid molecule of any one of claims 32-34, wherein designed saRNAs targeting the nucleic acid molecule are as defined in any one of claims 1-20; and/or
the desired saRNA is blunt ended or with an overhang, and/or without chemical modification(s) or with chemical modification(s).

37. An isolated polynucleotide encoding the saRNA of any one of claims 1-20 or the oligonucleotide modulator of any one of claims 21-26.

38. The isolated polynucleotide of claim 37, wherein the isolated polynucleotide is a DNA.

39. A vector comprising the isolated polynucleotide of any one of claims 37-38.

40. A host cell comprising the saRNA of any one of claims 1-20, the oligonucleotide modulator of any one of claims 21-26, the isolated polynucleotide of any one of claims 37-38, or the vector of claim 39.

41. A product comprising the saRNA of any one of claims 1-20, the oligonucleotide modulator of any one of claims 21-26, the isolated oligonucleotide of any one of claims 27-29, the oligonucleotide complex of any one of claims 30-31, the isolated nucleic acid molecule of any one of claims 32-36, the isolated polynucleotide of any one of claims 37-38, the vector of claim 39 or the host cell of claim 40.

42. The product of claim 41, wherein the product is a product for prevention, treatment or diagnosis of FVII-related disease or disorder or condition (such as a FVII defect associated disease or hemophilia), for saRNA designing and/or screening.

43. A composition comprising the saRNA of any one of claims 1-20, the oligonucleotide modulator of any one of claims 21-26, or the isolated polynucleotide of claim 37-38 and optionally, a pharmaceutically acceptable carrier.

44. The composition of claim 43, wherein the composition comprises 0.001-1600 nM, such as 1-150 nM of the saRNA.

45. A product for activating/up-regulating FVII gene expression in a cell, wherein the product activates the expression of FVII gene by at least 10% as compared to baseline expression of the FVII gene, and wherein the product comprises an active substance selected from one or more of the saRNA of any one of claims 1-20 or an oligonucleotide modulator of any one of claims 21-26, the isolated polynucleotide of any one of claims 37-38, the vector of claim 39, or the composition of any one of claims 43-44.

46. Use of an active substance in the preparation of a product for activating/up-regulating FVII gene expression in a cell, wherein the product activates the expression of FVII gene by at least 10% as compared to baseline expression of the FVII gene, and wherein the active substance is selected from one or more of the saRNA of any one of claims 1-20 or an oligonucleotide modulator of any one of claims 21-26, the isolated polynucleotide of any one of claims 37-38, the vector of claim 39, or the composition of any one of claims 43-44.

47. A method for activating/up-regulating FVII gene expression in a cell, wherein the product activates the expression of FVII gene by at least 10% as compared to baseline expression of the FVII gene, and wherein the method comprises administering an effective amount of an active substance to a cell, wherein the active substance is selected from one or more of the saRNA of any one of claims 1-20 or an oligonucleotide modulator of any one of claims 21-26, the isolated polynucleotide of any one of claims 37-38, the vector of claim 39, or the composition of any one of claims 41-44.

48. The product for activating/up-regulating FVII gene expression in a cell of claim 45, the use of claim 46 or the method of claim 47, wherein the active substance is introduced into the cell; and/or
wherein the cell is in vitro, ex vivo or in vivo; and/or
wherein the cell is a mammalian cell.

49. The product, the use or the method of claim 48, wherein the active substance is introduced into the cell by:
1) composing the active substance with a physiologically acceptable or pharmaceutically acceptable carrier, such as one or more selected from the group consisting of an aqueous carrier, a liposome, a high-molecular polymer, a polypeptide and an antibody, and/or
2) conjugating the active substance to one or more conjugation moieties, such as one or more selected from a lipid, a cell-penetrating peptide, a polyethylene glycol, an alkaloid, a tryptamine, a benzimidazole, a quinolone, an amino acid, a cholesterol, a glucose, and a N-acetylgalactosamine, and any combinations thereof (for example two conjugation moieties wherein one is a lipid and the other is a N-acetylgalactosamine).

50. The product, the use or the method of claim 49, wherein said conjugation moiety is one or more selected from S9, tC2, tC2x6, and C5x5, or any combinations thereof (such as C5x5 and tC2x6): 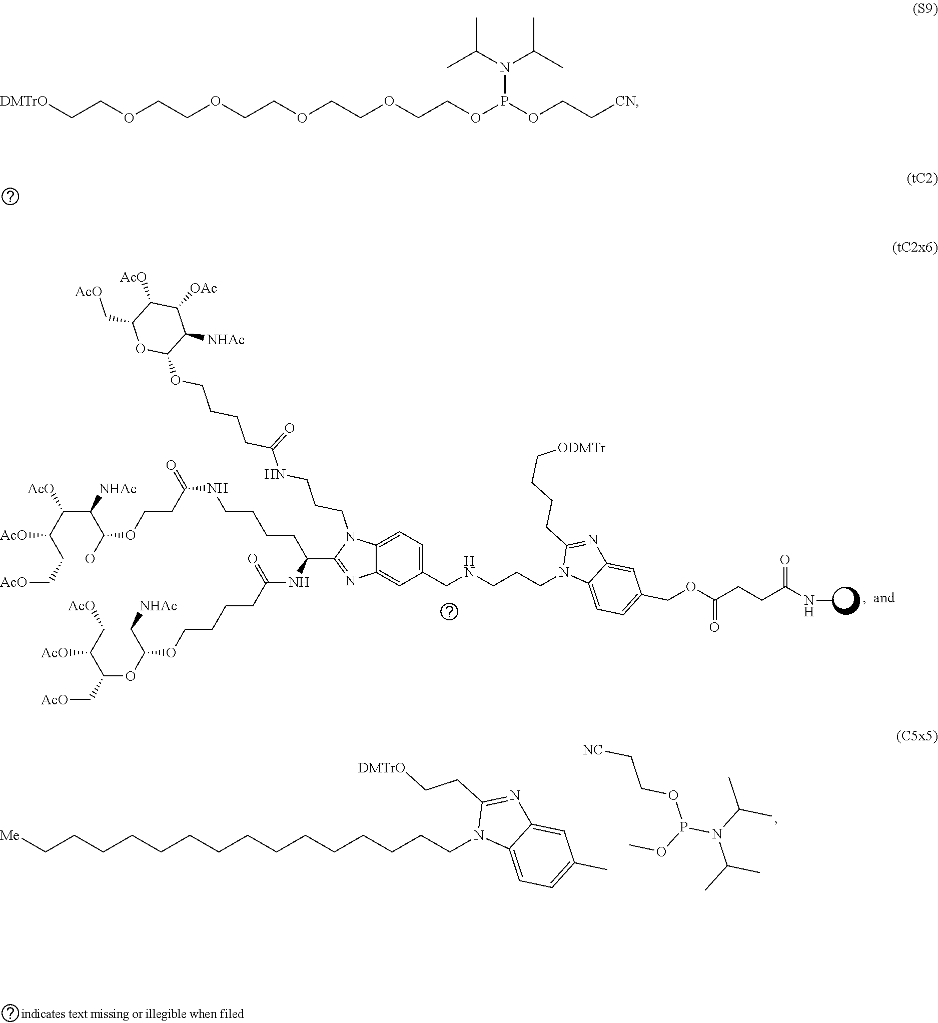
wherein [symbol] represents a support material.

51. The product, the use or the method of claim 49, wherein said conjugation moiety is a lipid selected from fatty acid comprising a carbon chain length of from 4-30, 12-24, 16-22 carbon atoms; and/or
wherein said conjugation moiety is a fatty acid having a carbon chain length of 4-30, 12-24, 16-22, or 16 carbon atoms; and/or
wherein the conjugation moiety is independently derived from a fluorophore, a ligand, a saccharide, a peptide, and an antibody.

52. The product for activating/up-regulating FVII gene expression in a cell of claim 45, the use of claim 46 or the method of claim 47, wherein the cell is from a patient suffering from or in risk of having a disease or condition or disorder induced by insufficient expression of the FVII protein, a FVII gene mutation, low functional FVII levels in blood, and/or other diseases or conditions or disorders preventable or treatable by activating/up-regulating FVII gene expression, such as hemophilia (e.g., hemophilia with inhibitor) or Glanzmann's thrombasthenia (GT), wherein the active substance is administered in a sufficient amount to prevent or treat the disease or condition or disorder.

53. A product for preventing or treating FVII-related disease or condition or disorder, wherein the product comprises an active substance selected from one or more of the saRNA of any one of claims 1-20 or an oligonucleotide modulator of any one of claims 21-26, the isolated polynucleotide of any one of claims 37-38, the vector of claim 39, or the composition of any one of claims 43-44.

54. Use of an active substance in the preparation of a product for preventing or treating FVII-related disease or condition or disorder, wherein the active substance is selected from one or more of the saRNA of any one of claims 1-20 or an oligonucleotide modulator of any one of claims 21-26, the isolated polynucleotide of any one of claims 37-38, the vector of claim 39, or the composition of any one of claims 43-44.

55. A method for preventing or treating FVII-related disease or condition or disorder, wherein the method comprises administering an effective amount of an active substance to a subject, wherein the active substance is selected from one or more of the saRNA of any one of claims 1-20 or an oligonucleotide modulator of any one of claims 21-26, the isolated polynucleotide of any one of claims 37-38, the vector of claim 39, or the composition of any one of claims 43-44.

56. The product of claim 53, the use of claim 54 or the method of claim 55, wherein the subject is a mammal (such as a human), preferably a mammal suffering from or in risk of having a disease or condition or disorder induced by insufficient expression of factor VII (FVII) protein, a FVII gene mutation, low functional FVII levels in blood and/or other diseases or conditions or disorders preventable or treatable by activating/up-regulating FVII level (such as hemophilia with inhibitor).

57. The product of claim 53, the use of claim 54 or the method of claim 55, wherein the active ingredient is administrated to the individual by an administration route selected from one or more of: parenteral infusions, oral administration, intranasal administration, inhaled administration, vaginal administration, and rectal administration.

58. The product of claim 53, the use of claim 54 or the method of claim 55, wherein the active ingredient is administrated to the individual by an administration route selected from one or more of intrathecal, intramuscular, intravenous, intraarterial, intraperitoneal, intravesical, intracerebroventricular, intravitreal and subcutaneous administrations.

59. The product of claim 53, the use of claim 54 or the method of claim 55, wherein the expression of the FVII gene mRNA in the individual is activates/up-regulates by at least 10% as compared to baseline expression of the FVII gene; and/or wherein the level of FVII protein in the individual is increased by at least 10% as compared to baseline level of FVII protein; and/or
wherein the FVII-related disease or condition or disorder is selected from the group consisting of congenital FVII deficiency (Alexander's Disease), Acquired FVII deficiency (AFVIID), hemophilia (such as hemophilia with inhibitor) and Glanzmann's thrombasthenia (GT).

60. A method for diagnosing FVII related disease or disorder by detecting FVII protein or FVII regulated protein in the cell of claim 40.

61. A kit for performing the method of claim 60, comprising the saRNA of any one of claims 1-20 or the oligonucleotide modulator of any one of claims 21-26.

62. The kit of claim 61, wherein the instruction for use comprising means for administering the saRNA of any one of claims 1-20 or the oligonucleotide modulator of any one of claims 21-26 to an individual.

63. A kit comprising the saRNA of any one of claims 1-20 or the oligonucleotide modulator of any one of claims 21-26, the isolated polynucleotide of any one of claims 37-38, the vector of claim 39, or the composition of any one of claims 43-44 in a labeled package and the label on package indicates that the saRNA, the isolated polynucleotide, the vector or the composition can be used in preventing or treating a disease or condition or disorder induced by insufficient expression of factor VII (FVII), or against hemophilia.

64. A kit for detecting FVII protein or FVII regulated protein in the cell of claim 40.

65. A Method for obtaining a saRNA capable of up-regulating the expression of FVII gene by at least 10% as compared to baseline expression of FVII gene, wherein the method comprises:
(a) synthesizing a saRNA comprising a sense strand and an antisense strand, wherein each of the sense strand or the antisense strand comprises a consecutive oligonucleotide sequence of 16 to 35 consecutive nucleotides, wherein the consecutive oligonucleotide sequence independently has at least 75%, at least 80%, at least 85%, at least 90%, at least 95%, at least 98% or 100% homology or complementarity to an equal length consecutive fragment of SEQ ID NO: 1437, and wherein the consecutive oligonucleotide sequence has (1) GC content between 40% and 70%; (2) no more than 5 consecutive identical nucleotides; (3) no more than 3 dinucleotide or trinucleotide repeats; and
(b) determining the capacity of the saRNA in up-regulating the expression of FVII gene.

66. A Method for obtaining a saRNA capable of up-regulating the expression of FVII gene by at least 10% as compared to baseline expression of FVII gene, wherein the method comprises:
(a) synthesizing a saRNA targeting a consecutive fragment of SEQ ID NO: 1437, and wherein the saRNA has (1) GC content between 40% and 70%; (2) no more than 5 consecutive identical nucleotides; (3) no more than 3 dinucleotide or trinucleotide repeats; and
(b) determining the capacity of the saRNA in up-regulating the expression of FVII gene.

67. The method of claim 65 or 66, wherein the consecutive fragment of SEQ ID NO: 1437 is located in a region upstream of the transcription start site (TSS) of FVII gene, and wherein
the consecutive fragment of SEQ ID NO: 1437 is located in a region selected from the group consisting of: region −557 to −379; region −346 to −298; region −271 to −91; and, region −96 to −1; and/or
the consecutive fragment of SEQ ID NO: 1437 is located in a region of SEQ ID NO: 1438, SEQ ID NO: 1439, SEQ ID NO: 1440, or SEQ ID NO: 1441.

68. The method of claim 65 or 66, wherein the consecutive fragment of SEQ ID NO: 1437 is selected from SEQ ID NO: 1-286.

69. A double-stranded functional saRNA molecule, wherein the functional RNA molecule is blunt-ended at both terminals; and/or
wherein each strand in the functional RNA molecule has a length of about 16 to about 35, about 17 to about 30, about 18 to about 25, or about 19 to about 22 nucleotides.

70. The double-stranded functional RNA molecule of claim 69, wherein the functional RNA molecule targets and regulates FVII gene expression.

71. An oligonucleotide agent comprising one or more saRNA according to any one of claims 69-70.

72. The oligonucleotide agent of claim 71, further comprising one or more moieties or components conjugated with said agent(s).

73. The oligonucleotide agent of claim 72, wherein the sense strand and/or the antisense strand of the functional saRNA is conjugated to one or more conjugation moieties selected from the group consisting of a lipid, a fatty acid (such as fatty acid comprising a carbon chain with 4-30, 12-24, 16-22 carbon atoms), a fluorophore, a ligand, a saccharide, a peptide, and an antibody.

74. The oligonucleotide agent of claim 73, wherein the conjugation moiety is independently selected from a lipid, a cell-penetrating peptide, a polyethylene glycol, an alkaloid, a tryptamine, a benzimidazole, a quinolone, an amino acid, a cholesterol, a glucose, a N-acetylgalactosamine, and any combinations thereof.

75. The oligonucleotide agent of claim 74, wherein the conjugation moieties is independently selected from S9, tC2, tC2x6, C5x5, and any combinations thereof, 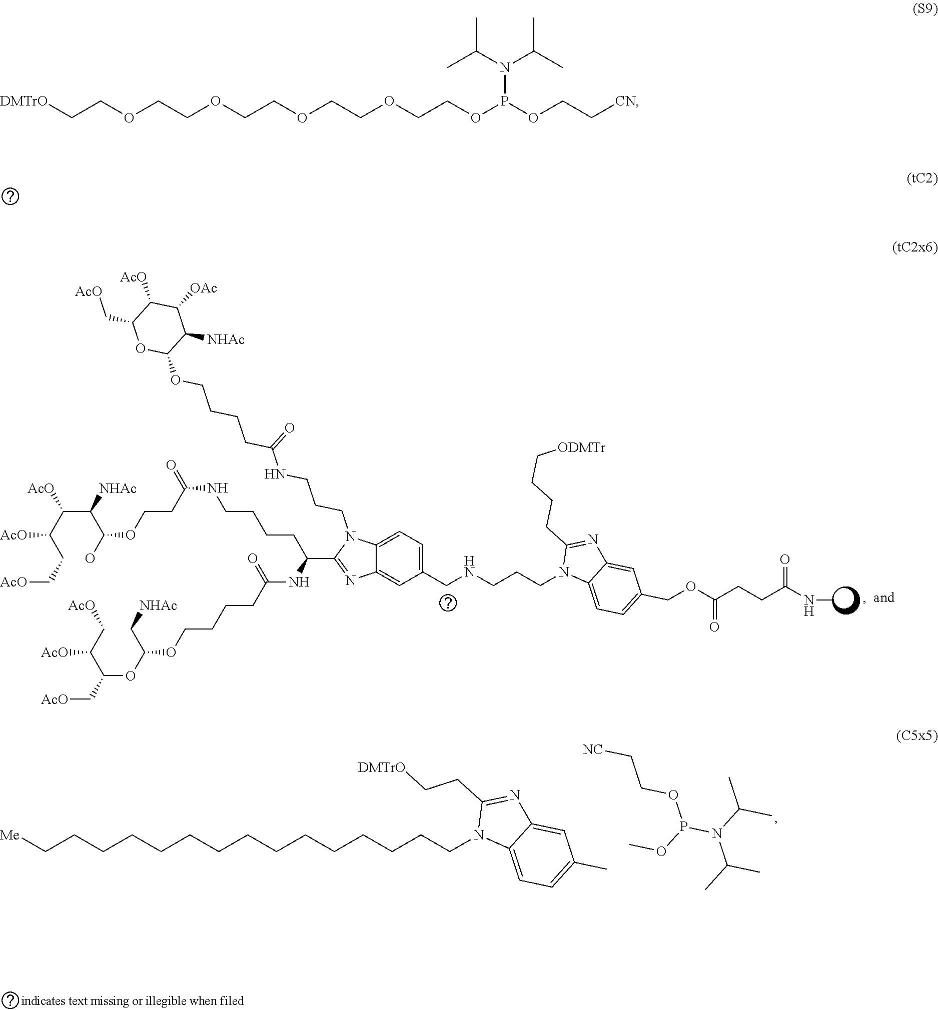
wherein [symbol] represents a support material.
